SEQUENCE LISTING (12 sequences)

SEQ ID NO: 1 - protein, 385 aa, Homo sapiens
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 2 - DNA, 1717 bp, Homo sapiens
aacgccagcgcctgcgcactgagggcggcctggtcgtcgtctgcggcggcggcggcggctgaggagcccggctgaggcgccagtacccggcccggtccgcatttcgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatggtaaaaaaacaaaacctaacactaaggatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttcaacaaaaaaaaaaaaaaa

SEQ ID NO: 3 - protein, 376 aa, Homo sapiens
MMMMALSKTFGQKPVKFQLEDDGEFYMIGSEVGNYLRMFRGSLYKRYPSLWRRLATVEERKKIVASSHDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 4 - DNA, 1690 bp, Homo sapiens
aacgccagcgcctgcgcactgagggcggcctggtcgtcgtctgcggcggcggcggcggctgaggagcccggctgaggcgccagtacccggcccggtccgcatttcgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatgatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttcaacaaaaaaaaaaaaaaa

SEQ ID NO: 5 - protein, 2492 aa, Homo sapiens
MTAEPMSESKLNTLVQKLHDFLAHSSEESEETSSPPRLAMNQNTDKISGSGSNSDMMENSKEEGTSSSEKSKSSGSSRSKRKPSIVTKYVESDDEKPLDDETVNEDASNENSENDITMQSLPKGTVIVQPEPVLNEDKDDFKGPEFRSRSKMKTENLKKRGEDGLHGIVSCTACGQQVNHFQKDSIYRHPSLQVLICKNCFKYYMSDDISRDSDGMDEQCRWCAEGGNLICCDFCHNAFCKKCILRNLGRKELSTIMDENNQWYCYICHPEPLLDLVTACNSVFENLEQLLQQNKKKIKVDSEKSNKVYEHTSRFSPKKTSSNCNGEEKKLDDSCSGSVTYSYSALIVPKEMIKKAKKLIETTANMNSSYVKFLKQATDNSEISSATKLRQLKAFKSVLADIKKAHLALEEDLNSEFRAMDAVNKEKNTKEHKVIDAKFETKARKGEKPCALEKKDISKSEAKLSRKQVDSEHMHQNVPTEEQRTNKSTGGEHKKSDRKEEPQYEPANTSEDLDMDIVSVPSSVPEDIFENLETAMEVQSSVDHQGDGSSGTEQEVESSSVKLNISSKDNRGGIKSKTTAKVTKELYVKLTPVSLSNSPIKGADCQEVPQDKDGYKSCGLNPKLEKCGLGQENSDNEHLVENEVSLLLEESDLRRSPRVKTTPLRRPTETNPVTSNSDEECNETVKEKQKLSVPVRKKDKRNSSDSAIDNPKPNKLPKSKQSETVDQNSDSDEMLAILKEVSRMSHSSSSDTDINEIHTNHKTLYDLKTQAGKDDKGKRKRKSSTSGSDFDTKKGKSAKSSIISKKKRQTQSESSNYDSELEKEIKSMSKIGAARTTKKRIPNTKDFDSSEDEKHSKKGMDNQGHKNLKTSQEGSSDDAERKQERETFSSAEGTVDKDTTIMELRDRLPKKQQASASTDGVDKLSGKEQSFTSLEVRKVAETKEKSKHLKTKTCKKVQDGLSDIAEKFLKKDQSDETSEDDKKQSKKGTEEKKKPSDFKKKVIKMEQQYESSSDGTEKLPEREEICHFPKGIKQIKNGTTDGEKKSKKIRDKTSKKKDELSDYAEKSTGKGDSCDSSEDKKSKNGAYGREKKRCKLLGKSSRKRQDCSSSDTEKYSMKEDGCNSSDKRLKRIELRERRNLSSKRNTKEIQSGSSSSDAEESSEDNKKKKQRTSSKKKAVIVKEKKRNSLRTSTKRKQADITSSSSSDIEDDDQNSIGEGSSDEQKIKPVTENLVLSSHTGFCQSSGDEALSKSVPVTVDDDDDDNDPENRIAKKMLLEEIKANLSSDEDGSSDDEPEEGKKRTGKQNEENPGDEEAKNQVNSESDSDSEESKKPRYRHRLLRHKLTVSDGESGEEKKTKPKEHKEVKGRNRRKVSSEDSEDSDFQESGVSEEVSESEDEQRPRTRSAKKAELEENQRSYKQKKKRRRIKVQEDSSSENKSNSEEEEEEKEEEEEEEEEEEEEEEDENDDSKSPGKGRKKIRKILKDDKLRTETQNALKEEEERRKRIAEREREREKLREVIEIEDASPTKCPITTKLVLDEDEETKEPLVQVHRNMVIKLKPHQVDGVQFMWDCCCESVKKTKKSPGSGCILAHCMGLGKTLQVVSFLHTVLLCDKLDFSTALVVCPLNTALNWMNEFEKWQEGLKDDEKLEVSELATVKRPQERSYMLQRWQEDGGVMIIGYEMYRNLAQGRNVKSRKLKEIFNKALVDPGPDFVVCDEGHILKNEASAVSKAMNSIRSRRRIILTGTPLQNNLIEYHCMVNFIKENLLGSIKEFRNRFINPIQNGQCADSTMVDVRVMKKRAHILYEMLAGCVQRKDYTALTKFLPPKHEYVLAVRMTSIQCKLYQYYLDHLTGVGNNSEGGRGKAGAKLFQDFQMLSRIWTHPWCLQLDYISKENKGYFDEDSMDEFIASDSDETSMSLSSDDYTKKKKKGKKGKKDSSSSGSGSDNDVEVIKVWNSRSRGGGEGNVDETGNNPSVSLKLEESKATSSSNPSSPAPDWYKDFVTDADAEVLEHSGKMVLLFEILRMAEEIGDKVLVFSQSLISLDLIEDFLELASREKTEDKDKPLIYKGEGKWLRNIDYYRLDGSTTAQSRKKWAEEFNDETNVRGRLFIISTKAGSLGINLVAANRVIIFDASWNPSYDIQSIFRVYRFGQTKPVYVYRFLAQGTMEDKIYDRQVTKQSLSFRVVDQQQVERHFTMNELTELYTFEPDLLDDPNSEKKKKRDTPMLPKDTILAELLQIHKEHIVGYHEHDSLLDHKEEEELTEEERKAAWAEYEAEKKGLTMRFNIPTGTNLPPVSFNSQTPYIPFNLGALSAMSNQQLEDLINQGREKVVEATNSVTAVRIQPLEDIISAVWKENMNLSEAQVQALALSRQASQELDVKRREAIYNDVLTKQQMLISCVQRILMNRRLQQQYNQQQQQQMTYQQATLGHLMMPKPPNLIMNPSNYQQIDMRGMYQPVAGGMQPPPLQRAPPPMRSKNPGPSQGKSM

SEQ ID NO: 6 - DNA, 11202 bp, Homo sapiens
aattctcctgcctgagcctcggcccaacaaaatggcggcggcagcggtgtcgctttgtttccgcggctcctgcggcggtggcagtggtagcggcctttgagctgtggggaggttccagcagcagctacagtgacgactaagactccagtgcatttctatcgtaaccgggcgcgggggagcgcagatcggcgcccagcaatcacagaagccgacaaggcgttcaagcgaaaacatgaccgctgagcccatgagtgaaagcaagttgaatacattggtgcagaagcttcatgacttccttgcacactcatcagaagaatctgaagaaacaagttctcctccacgacttgcaatgaatcaaaacacagataaaatcagtggttctggaagtaactctgatatgatggaaaacagcaaggaagagggaactagctcttcagaaaaatccaagtcttcaggatcgtcacgatcaaagaggaaaccttcaattgtaacaaagtatgtagaatcagatgatgaaaaacctttggatgatgaaactgtaaatgaagatgcgtctaatgaaaattcagaaaatgatattactatgcagagcttgccaaaaggtacagtgattgtacagccagagccagtgctgaatgaagacaaagatgattttaaagggcctgaatttagaagcagaagtaaaatgaaaactgaaaatctcaaaaaacgcggagaagatgggcttcatgggattgtgagctgcactgcttgtggacaacaggtcaatcattttcaaaaagattccatttatagacacccttcattgcaagttcttatttgtaagaattgctttaagtattacatgagtgatgatattagccgtgactcagatggaatggatgaacaatgtaggtggtgtgcggaaggtggaaacttgatttgttgtgacttttgccataatgctttctgcaagaaatgcattctacgcaaccttggtcgaaaggagttgtccacaataatggatgaaaacaaccaatggtattgctacatttgtcacccagagcctttgttggacttggtcactgcatgtaacagcgtatttgagaatttagaacagttgttgcagcaaaataagaagaagataaaagttgacagtgaaaagagtaataaagtatatgaacatacatccagattttctccaaagaagactagttcaaattgtaatggagaagaaaagaaattagatgattcctgttctggctctgtaacctactcttattccgcactaattgtgcccaaagagatgattaagaaggcaaaaaaactgattgagaccacagccaacatgaactccagttatgttaaatttttaaagcaggcaacagataattcagaaatcagttctgctacaaaattacgtcagcttaaggcttttaagtctgtgttggctgatattaagaaggctcatcttgcattggaagaagacttaaattccgagtttcgagcgatggatgctgtaaacaaagagaaaaataccaaagagcataaagtcatagatgctaagtttgaaacaaaagcacgaaaaggagaaaaaccttgtgctttggaaaagaaggatatttcaaagtcagaagctaaactttcaagaaaacaggtagatagtgagcacatgcatcagaatgttccaacagaggaacaaagaacaaataaaagtaccggtggtgaacataagaaatctgatagaaaagaagaacctcaatatgaacctgccaacacttctgaagatttagacatggatattgtgtctgttccttcctcagttccagaagacatttttgagaatcttgagactgctatggaagttcagagttcagttgatcatcaaggggatggcagcagtggaactgaacaagaagtggagagttcatctgtaaaattaaatatttcttcaaaagacaacagaggaggtattaaatcaaaaactacagctaaagtaacaaaagaattatatgttaaactcactcctgtttccctttctaattccccaattaaaggtgctgattgtcaggaagttccacaagataaagatggctataaaagttgtggtctgaaccccaagttagagaaatgtggacttggacaggaaaacagtgataatgagcatttggttgaaaatgaagtttcattacttttagaggaatctgatcttcgaagatccccacgtgtaaagactacacccttgaggcgaccgacagaaactaaccctgtaacatctaattcagatgaagaatgtaatgaaacagttaaggagaaacaaaaactatcagttccagtgagaaaaaaggataagcgtaattcttctgacagtgctatagataatcctaagcctaataaattgccaaaatctaagcaatcagagactgtggatcaaaattcagattctgatgaaatgctagcaatcctcaaagaggtgagcaggatgagtcacagttcttcttcagatactgatattaatgaaattcatacaaaccataagactttgtatgatttaaagactcaggcggggaaagatgataaaggaaaaaggaaacgaaaaagttctacatctggctcagattttgatactaaaaagggcaaatcagctaagagctctataatttctaaaaagaaacgacaaacccagtctgagtcttctaattatgactcagaattagaaaaagagataaagagcatgagtaaaattggtgctgccagaaccaccaaaaaaagaattccaaatacaaaagattttgactcttctgaagatgagaaacacagcaaaaaaggaatggataatcaagggcacaaaaatttgaagacctcacaagaaggatcatctgatgatgctgaaagaaaacaagagagagagactttctcttcagcagaaggcacagttgataaagacacgaccatcatggaattaagagatcgacttcctaagaagcagcaagcaagtgcttccactgatggtgtcgataagctttctgggaaagagcagagttttacttctttggaagttagaaaagttgctgaaactaaagaaaagagcaagcatctcaaaaccaaaacatgtaaaaaagtacaggatggcttatctgatattgcagagaaattcctaaagaaagaccagagcgatgaaacttctgaagatgataaaaagcagagcaaaaagggaactgaagaaaaaaagaaaccttcagactttaagaaaaaagtaattaaaatggaacaacagtatgaatcttcatctgatggcactgaaaagttacctgagcgagaagaaatttgtcattttcctaagggcataaaacaaattaagaatggaacaactgatggagaaaagaaaagtaaaaaaataagagataaaacttctaaaaagaaggatgaattatctgattatgctgagaagtcaacagggaaaggagatagttgtgactcttcagaggataaaaagagtaagaatggagcatatggtagagagaagaaaaggtgcaagttgcttggaaagagttcaaggaagagacaagattgttcatcatctgatactgagaaatattccatgaaagaagatggttgtaactcttctgataagagactgaaaagaatagaattgagggaaagaagaaatttaagttcaaagagaaatactaaggaaatacaaagtggctcatcatcatctgatgctgaggaaagttctgaagataataaaaagaagaagcaaagaacttcatctaaaaagaaggcagtcattgtcaaggagaaaaagagaaactccctaagaacaagcactaaaaggaagcaagctgacattacatcctcatcttcttctgatatagaagatgatgatcagaattctataggtgagggaagcagcgatgaacagaaaattaagcctgtgactgaaaatttagtgctgtcttcacatactggattttgccaatcttcaggagatgaagccttatctaaatcagtgcctgtcacagtggatgatgatgatgacgacaatgatcctgagaatagaattgccaagaagatgcttttagaagaaattaaagccaatctttcctctgatgaggatggatcttcagatgatgagccagaagaagggaaaaaaagaactggaaaacaaaatgaagaaaacccaggagatgaggaagcaaaaaatcaagtcaattctgaatcagattcagattctgaagaatctaagaagccaagatacagacataggcttttgcggcacaaattgactgtgagtgacggagaatctggagaagaaaaaaagacaaagcctaaagagcataaagaagtcaaaggcagaaacagaagaaaggtgagcagtgaagattcagaagattctgattttcaggaatcaggagttagtgaagaagttagtgaatccgaagatgaacagcggcccagaacaaggtctgcaaagaaagcagagttggaagaaaatcagcggagctataaacagaaaaagaaaaggcgacgtattaaggttcaagaagattcatccagtgaaaacaagagtaattctgaggaagaagaggaggaaaaagaagaggaggaggaagaggaggaggaggaggaagaggaggaggaagatgaaaatgatgattccaagtctcctggaaaaggcagaaagaaaattcggaagattcttaaagatgataaactgagaacagaaacacaaaatgctcttaaggaagaggaagagagacgaaaacgtattgctgagagggagcgtgagcgagaaaaattgagagaggtgatagaaattgaagatgcttcacccaccaagtgtccaataacaaccaagttggttttagatgaagatgaagaaaccaaagaacctttagtgcaggttcatagaaatatggttatcaaattgaaaccccatcaagtagatggtgttcagtttatgtgggattgctgctgtgagtctgtgaaaaaaacaaagaaatctccaggttcaggatgcattcttgcccactgtatgggccttggtaagactttacaggtggtaagttttcttcatacagttcttttgtgtgacaaactggatttcagcacggcgttagtggtttgtcctcttaatactgctttgaattggatgaatgaatttgagaagtggcaagagggattaaaagatgatgagaagcttgaggtttctgaattagcaactgtgaaacgtcctcaggagagaagctacatgctgcagaggtggcaagaagatggtggtgttatgatcataggctatgagatgtatagaaatcttgctcaaggaaggaatgtgaagagtcggaaacttaaagaaatatttaacaaagctttggttgatccaggccctgattttgttgtttgtgatgaaggccatattctaaaaaatgaagcatctgctgtttctaaagctatgaattctatacgatcaaggaggaggattattttaacaggaacaccacttcaaaataacctaattgagtatcattgtatggttaattttatcaaggaaaatttacttggatccattaaggagttcaggaatagatttataaatccaattcaaaatggtcagtgtgcagattctaccatggtagatgtcagagtgatgaaaaaacgtgctcacattctctatgagatgttagctggatgtgttcagaggaaagattatacagcattaacaaaattcttgcctccaaaacacgaatatgtgttagctgtgagaatgacttctattcagtgcaagctctatcagtactacttagatcacttaacaggtgtgggcaataatagtgaaggtggaagaggaaaggcaggtgcaaagcttttccaagattttcagatgttaagtagaatatggactcatccttggtgtttgcagctagactacattagcaaagaaaataagggttattttgatgaagacagtatggatgaatttatagcctcagattctgatgaaacctccatgagtttaagctccgatgattatacaaaaaagaagaaaaaagggaaaaaggggaaaaaagatagtagctcaagtggaagtggcagtgacaatgatgttgaagtgattaaggtctggaattcaagatctcggggaggtggtgaaggaaatgtggatgaaacaggaaacaatccttctgtttctttaaaactggaagaaagtaaagctacttcttcttctaatccaagcagcccagctccagactggtacaaagattttgttacagatgctgatgctgaggttttagagcattctgggaaaatggtacttctctttgaaattcttcgaatggcagaggaaattggggataaagtccttgttttcagccagtccctcatatctctggacttgattgaagattttcttgaattagctagtagggagaagacagaagataaagataaaccccttatttataaaggtgaggggaagtggcttcgaaacattgactattaccgtttagatggttccactactgcacagtcaaggaagaagtgggctgaagaatttaatgatgaaactaatgtgagaggacgattatttatcatttctactaaagcaggatctctaggaattaatctggtagctgctaatcgagtaattatattcgacgcttcttggaatccatcttatgacatccagagtatattcagagtttatcgctttggacaaactaagcctgtttatgtatataggttcttagctcagggaaccatggaagataagatttatgatcggcaagtaactaagcagtcactgtcttttcgagttgttgatcagcagcaggtggagcgtcattttactatgaatgagcttactgaactttatacttttgagccagacttattagatgaccctaattcagaaaagaagaagaagagggatactcccatgctgccaaaggataccatacttgcagagctccttcagatacataaagaacacattgtaggataccatgaacatgattctcttttggaccacaaagaagaagaagagttgactgaagaagaaagaaaagcagcttgggctgagtatgaagcagagaagaagggactgaccatgcgtttcaacataccaactgggaccaatttaccccctgtcagtttcaactctcaaactccttatattcctttcaatttgggagccctgtcagcaatgagtaatcaacagctggaggacctcattaatcaaggaagagaaaaagttgtagaagcaacaaacagtgtgacagcagtgaggattcaacctcttgaggatataatttcagctgtatggaaggagaacatgaatctctcagaggcccaagtacaggcgttagcattaagtagacaagccagccaggagcttgatgttaaacgaagagaagcaatctacaatgatgtattgacaaaacaacagatgttaatcagctgtgttcagcgaatacttatgaacagaaggctccagcagcagtacaatcagcagcaacagcaacaaatgacttatcaacaagcaacactgggtcacctcatgatgccaaagcccccaaatttgatcatgaatccttctaactaccagcagattgatatgagaggaatgtatcagccagtggctggtggtatgcagccaccaccattacagcgtgcaccacccccaatgagaagcaaaaatccaggaccttcccaagggaaatcaatgtgattttgcactaaaagcttaatggattgttaaaatcatagaaagatcttttatttttttaggaatcaatgacttaacagaactcaactgtataaatagtttggtccccttaaatgccaatcttccatattagttttactttttttttttttaaatagggcataccatttcttcctgacatttgtcagtgatgttgcctagaatcttcttacacacgctgagtacagaagatatttcaaattgttttcagtgaaaacaagtccttccataatagtaacaactccacagatttcctctctaaatttttatgcctgcttttagcaaccataaaattgtcataaaattaataaatttaggaaagaataaagatttatatattcattctttacatataaaaacacacagctgagttcttagagttgattcctcaagttatgaaatacttttgtacttaatccatttcttgattaaagtgattgaaatggttttaatgttcttttgactgaagtctgaaactgggctcctgctttattgtctctgtgactgaaagttagaaactgagggttatctttgacacagaattgtgtgcaatattcttaaatactactgctctaaaagttggagaagtcttgcagttatcttagcattgtataaacagccttaagtatagcctaagaagagaattcctttttcttctttagtccttctgccattttttattttcagttatatgtgctgaaataattactggtaaaatttcagggttgtggattatcttccacacatgaattttctctctcctggcacgaatataaagcacatctcttaactgcatggtgccagtgctaatgcttcatcctgttgctggcagtgggatgtggacttagaaaatcaagttctagcattttagtaggttaacactgaagttgtggttgttaggttcacaccctgttttataaacaacatcaaaatggcagaaccattgctgactttaggttcacatgaggaatgtacttttaacaattcccagtactatcagtattgtgaaataattcctctgaaagataagaatcactggcttctatgcgcttcttttctctcatcatcatgttcttttaccccagtttccttacatttttttaaattgtttcagagtttgttttttttttagtttagattgtgaggcaattattaaatcaaaattaattcatccaatacccctttactagaagttttactagaaaatgtattacattttattttttcttaatccagttctgcaaaaatgacctataaatttattcatgtacaattttggttacttgaattgttaaagaaaacattgtttttgactatgggagtcaactcaacatggcagaaccatttttgagatgatgatacaacaggtagtgaaacagcttaagaattccaaaaaaaaaaaaaaaaaaaaaaaaaagaaaactgggtttgggctttgctttaggtatcactggattagaatgagtttaacattagctaaaactgctttgagttgtttggatgattaagagattgccatttttatcttggaagaactagtggtaaaacatccaagagcactaggattgtgatacagaatttgtgaggtttggtggatccacgcccctctcccccactttcccatgatgaaatatcactaataaatcctgtatatttagatattatgctagccatgtaatcagatttatttaattgggtggggcaggtgtgtatttactttagaaaaaatgaaaaagacaagatttatgagaaatatttgaaggcagtacactctggccaactgttaccagttggtatttctacaagttcagaatattttaaacctgatttactagacctgggaattttcaacatggtctaattatttactcaaagacatagatgtgaaaattttaggcaaccttctaaatctttttcaccatggatgaaactataacttaaagaataatacttagaagggttaattggaaatcagagtttgaaataaaacttggaccactttgtatacactcttctcacttgacattttagctatataatatgtactttgagtataacatcaagctttaacaaatatttaaagacaaaaaaatcacgtcagtaaaatactaaaaggctcatttttatatttgttttagatgttttaaatagttgcaatggattaaaaatgatgatttaaaatgttgcttgtaatacagttttgcctgctaaattctccacattttgtaacctgttttatttctttgggtgtaaagcgtttttgcttagtattgtgatattgtatatgttttgtcccagttgtatagtaatgtttcagtccatcatccagctttggctgctgaaatcatacagctgtgaagacttgcctttgtttctgttagactgcttttcagttctgtattgagtatcttaagtactgtagaaaagatgtcacttcttcctttaaggctgttttgtaatatatataaggactggaattgtgtttttaaagaaaagcattcaagtatgacaatatactatctgtgttttcaccattcaaagtgctgtttagtagttgaaacttaaactatttaatgtcatttaataaagtgaccaaaatgtgttgtgctctttattgtattttcacagctttgaaaatctgtgcacatactgtttcatagaaaatgtatagcttttgttgtcctatataatggtggttcttttgcacatttagttatttaatattgagaggtcacgaagtttggttattgaatctgttatatactaaattctgtaaagggagatctctcatctcaaaaagaatttacataccaggaagtccatgtgtgtttgtgttagttttggatgtctttgtgtaatccagccccatttcctgtttcccaacagctgtaacactcattttaagtcaagcagggctaccaacccacacttgatagaaaagctgcttaccattcagaagcttccttattacctggcctccaaatgagctgaatattttgtagccttcccttagctatgttcattttccctccattatcataaaatcagatcgatatttatgtgccccaaacaaaactttaagagcagttacattctgtcccagtagcccttgtttcctttgagagtagcatgttgtgaggctatagagacttattctaccagtaaaacaggtcaatccttttacatgtttattatactaaaaattatgttcagggtatttactactttatttcaccagactcagtctcaagtgacttggctatctccaaatcagatctacccttagagaataaacatttttctaccgttattttttttcaagtctataatctgagccagtcccaaaggagtgatcaagtttcagaaatgctttcatcttcacaacattttatatatactattatatggggtgaataaagttttaaatccgaaatataaaaaaaaaaaaaaaaaaa

SEQ ID NO: 7 - protein, 2454 aa, Homo sapiens
MTAEPMSESKLNTLVQKLHDFLAHSSEESEETSSPPRLAMNQNTDKISGSGSNSDMMENSKEEGTSSSEKSKSSGSSRSKRKPSIVTKYVESDDEKPLDDETVNEDASNENSENDITMQSLPKEDGLHGIVSCTACGQQVNHFQKDSIYRHPSLQVLICKNCFKYYMSDDISRDSDGMDEQCRWCAEGGNLICCDFCHNAFCKKCILRNLGRKELSTIMDENNQWYCYICHPEPLLDLVTACNSVFENLEQLLQQNKKKIKVDSEKSNKVYEHTSRFSPKKTSSNCNGEEKKLDDSCSGSVTYSYSALIVPKEMIKKAKKLIETTANMNSSYVKFLKQATDNSEISSATKLRQLKAFKSVLADIKKAHLALEEDLNSEFRAMDAVNKEKNTKEHKVIDAKFETKARKGEKPCALEKKDISKSEAKLSRKQVDSEHMHQNVPTEEQRTNKSTGGEHKKSDRKEEPQYEPANTSEDLDMDIVSVPSSVPEDIFENLETAMEVQSSVDHQGDGSSGTEQEVESSSVKLNISSKDNRGGIKSKTTAKVTKELYVKLTPVSLSNSPIKGADCQEVPQDKDGYKSCGLNPKLEKCGLGQENSDNEHLVENEVSLLLEESDLRRSPRVKTTPLRRPTETNPVTSNSDEECNETVKEKQKLSVPVRKKDKRNSSDSAIDNPKPNKLPKSKQSETVDQNSDSDEMLAILKEVSRMSHSSSSDTDINEIHTNHKTLYDLKTQAGKDDKGKRKRKSSTSGSDFDTKKGKSAKSSIISKKKRQTQSESSNYDSELEKEIKSMSKIGAARTTKKRIPNTKDFDSSEDEKHSKKGMDNQGHKNLKTSQEGSSDDAERKQERETFSSAEGTVDKDTTIMELRDRLPKKQQASASTDGVDKLSGKEQSFTSLEVRKVAETKEKSKHLKTKTCKKVQDGLSDIAEKFLKKDQSDETSEDDKKQSKKGTEEKKKPSDFKKKVIKMEQQYESSSDGTEKLPEREEICHFPKGIKQIKNGTTDGEKKSKKIRDKTSKKKDELSDYAEKSTGKGDSCDSSEDKKSKNGAYGREKKRCKLLGKSSRKRQDCSSSDTEKYSMKEDGCNSSDKRLKRIELRERRNLSSKRNTKEIQSGSSSSDAEESSEDNKKKKQRTSSKKKAVIVKEKKRNSLRTSTKRKQADITSSSSSDIEDDDQNSIGEGSSDEQKIKPVTENLVLSSHTGFCQSSGDEALSKSVPVTVDDDDDDNDPENRIAKKMLLEEIKANLSSDEDGSSDDEPEEGKKRTGKQNEENPGDEEAKNQVNSESDSDSEESKKPRYRHRLLRHKLTVSDGESGEEKKTKPKEHKEVKGRNRRKVSSEDSEDSDFQESGVSEEVSESEDEQRPRTRSAKKAELEENQRSYKQKKKRRRIKVQEDSSSENKSNSEEEEEEKEEEEEEEEEEEEEEEDENDDSKSPGKGRKKIRKILKDDKLRTETQNALKEEEERRKRIAEREREREKLREVIEIEDASPTKCPITTKLVLDEDEETKEPLVQVHRNMVIKLKPHQVDGVQFMWDCCCESVKKTKKSPGSGCILAHCMGLGKTLQVVSFLHTVLLCDKLDFSTALVVCPLNTALNWMNEFEKWQEGLKDDEKLEVSELATVKRPQERSYMLQRWQEDGGVMIIGYEMYRNLAQGRNVKSRKLKEIFNKALVDPGPDFVVCDEGHILKNEASAVSKAMNSIRSRRRIILTGTPLQNNLIEYHCMVNFIKENLLGSIKEFRNRFINPIQNGQCADSTMVDVRVMKKRAHILYEMLAGCVQRKDYTALTKFLPPKHEYVLAVRMTSIQCKLYQYYLDHLTGVGNNSEGGRGKAGAKLFQDFQMLSRIWTHPWCLQLDYISKENKGYFDEDSMDEFIASDSDETSMSLSSDDYTKKKKKGKKGKKDSSSSGSGSDNDVEVIKVWNSRSRGGGEGNVDETGNNPSVSLKLEESKATSSSNPSSPAPDWYKDFVTDADAEVLEHSGKMVLLFEILRMAEEIGDKVLVFSQSLISLDLIEDFLELASREKTEDKDKPLIYKGEGKWLRNIDYYRLDGSTTAQSRKKWAEEFNDETNVRGRLFIISTKAGSLGINLVAANRVIIFDASWNPSYDIQSIFRVYRFGQTKPVYVYRFLAQGTMEDKIYDRQVTKQSLSFRVVDQQQVERHFTMNELTELYTFEPDLLDDPNSEKKKKRDTPMLPKDTILAELLQIHKEHIVGYHEHDSLLDHKEEEELTEEERKAAWAEYEAEKKGLTMRFNIPTGTNLPPVSFNSQTPYIPFNLGALSAMSNQQLEDLINQGREKVVEATNSVTAVRIQPLEDIISAVWKENMNLSEAQVQALALSRQASQELDVKRREAIYNDVLTKQQMLISCVQRILMNRRLQQQYNQQQQQQMTYQQATLGHLMMPKPPNLIMNPSNYQQIDMRGMYQPVAGGMQPPPLQRAPPPMRSKNPGPSQGKSM

SEQ ID NO: 8 - DNA, 11088 bp, Homo sapiens
aattctcctgcctgagcctcggcccaacaaaatggcggcggcagcggtgtcgctttgtttccgcggctcctgcggcggtggcagtggtagcggcctttgagctgtggggaggttccagcagcagctacagtgacgactaagactccagtgcatttctatcgtaaccgggcgcgggggagcgcagatcggcgcccagcaatcacagaagccgacaaggcgttcaagcgaaaacatgaccgctgagcccatgagtgaaagcaagttgaatacattggtgcagaagcttcatgacttccttgcacactcatcagaagaatctgaagaaacaagttctcctccacgacttgcaatgaatcaaaacacagataaaatcagtggttctggaagtaactctgatatgatggaaaacagcaaggaagagggaactagctcttcagaaaaatccaagtcttcaggatcgtcacgatcaaagaggaaaccttcaattgtaacaaagtatgtagaatcagatgatgaaaaacctttggatgatgaaactgtaaatgaagatgcgtctaatgaaaattcagaaaatgatattactatgcagagcttgccaaaagaagatgggcttcatgggattgtgagctgcactgcttgtggacaacaggtcaatcattttcaaaaagattccatttatagacacccttcattgcaagttcttatttgtaagaattgctttaagtattacatgagtgatgatattagccgtgactcagatggaatggatgaacaatgtaggtggtgtgcggaaggtggaaacttgatttgttgtgacttttgccataatgctttctgcaagaaatgcattctacgcaaccttggtcgaaaggagttgtccacaataatggatgaaaacaaccaatggtattgctacatttgtcacccagagcctttgttggacttggtcactgcatgtaacagcgtatttgagaatttagaacagttgttgcagcaaaataagaagaagataaaagttgacagtgaaaagagtaataaagtatatgaacatacatccagattttctccaaagaagactagttcaaattgtaatggagaagaaaagaaattagatgattcctgttctggctctgtaacctactcttattccgcactaattgtgcccaaagagatgattaagaaggcaaaaaaactgattgagaccacagccaacatgaactccagttatgttaaatttttaaagcaggcaacagataattcagaaatcagttctgctacaaaattacgtcagcttaaggcttttaagtctgtgttggctgatattaagaaggctcatcttgcattggaagaagacttaaattccgagtttcgagcgatggatgctgtaaacaaagagaaaaataccaaagagcataaagtcatagatgctaagtttgaaacaaaagcacgaaaaggagaaaaaccttgtgctttggaaaagaaggatatttcaaagtcagaagctaaactttcaagaaaacaggtagatagtgagcacatgcatcagaatgttccaacagaggaacaaagaacaaataaaagtaccggtggtgaacataagaaatctgatagaaaagaagaacctcaatatgaacctgccaacacttctgaagatttagacatggatattgtgtctgttccttcctcagttccagaagacatttttgagaatcttgagactgctatggaagttcagagttcagttgatcatcaaggggatggcagcagtggaactgaacaagaagtggagagttcatctgtaaaattaaatatttcttcaaaagacaacagaggaggtattaaatcaaaaactacagctaaagtaacaaaagaattatatgttaaactcactcctgtttccctttctaattccccaattaaaggtgctgattgtcaggaagttccacaagataaagatggctataaaagttgtggtctgaaccccaagttagagaaatgtggacttggacaggaaaacagtgataatgagcatttggttgaaaatgaagtttcattacttttagaggaatctgatcttcgaagatccccacgtgtaaagactacacccttgaggcgaccgacagaaactaaccctgtaacatctaattcagatgaagaatgtaatgaaacagttaaggagaaacaaaaactatcagttccagtgagaaaaaaggataagcgtaattcttctgacagtgctatagataatcctaagcctaataaattgccaaaatctaagcaatcagagactgtggatcaaaattcagattctgatgaaatgctagcaatcctcaaagaggtgagcaggatgagtcacagttcttcttcagatactgatattaatgaaattcatacaaaccataagactttgtatgatttaaagactcaggcggggaaagatgataaaggaaaaaggaaacgaaaaagttctacatctggctcagattttgatactaaaaagggcaaatcagctaagagctctataatttctaaaaagaaacgacaaacccagtctgagtcttctaattatgactcagaattagaaaaagagataaagagcatgagtaaaattggtgctgccagaaccaccaaaaaaagaattccaaatacaaaagattttgactcttctgaagatgagaaacacagcaaaaaaggaatggataatcaagggcacaaaaatttgaagacctcacaagaaggatcatctgatgatgctgaaagaaaacaagagagagagactttctcttcagcagaaggcacagttgataaagacacgaccatcatggaattaagagatcgacttcctaagaagcagcaagcaagtgcttccactgatggtgtcgataagctttctgggaaagagcagagttttacttctttggaagttagaaaagttgctgaaactaaagaaaagagcaagcatctcaaaaccaaaacatgtaaaaaagtacaggatggcttatctgatattgcagagaaattcctaaagaaagaccagagcgatgaaacttctgaagatgataaaaagcagagcaaaaagggaactgaagaaaaaaagaaaccttcagactttaagaaaaaagtaattaaaatggaacaacagtatgaatcttcatctgatggcactgaaaagttacctgagcgagaagaaatttgtcattttcctaagggcataaaacaaattaagaatggaacaactgatggagaaaagaaaagtaaaaaaataagagataaaacttctaaaaagaaggatgaattatctgattatgctgagaagtcaacagggaaaggagatagttgtgactcttcagaggataaaaagagtaagaatggagcatatggtagagagaagaaaaggtgcaagttgcttggaaagagttcaaggaagagacaagattgttcatcatctgatactgagaaatattccatgaaagaagatggttgtaactcttctgataagagactgaaaagaatagaattgagggaaagaagaaatttaagttcaaagagaaatactaaggaaatacaaagtggctcatcatcatctgatgctgaggaaagttctgaagataataaaaagaagaagcaaagaacttcatctaaaaagaaggcagtcattgtcaaggagaaaaagagaaactccctaagaacaagcactaaaaggaagcaagctgacattacatcctcatcttcttctgatatagaagatgatgatcagaattctataggtgagggaagcagcgatgaacagaaaattaagcctgtgactgaaaatttagtgctgtcttcacatactggattttgccaatcttcaggagatgaagccttatctaaatcagtgcctgtcacagtggatgatgatgatgacgacaatgatcctgagaatagaattgccaagaagatgcttttagaagaaattaaagccaatctttcctctgatgaggatggatcttcagatgatgagccagaagaagggaaaaaaagaactggaaaacaaaatgaagaaaacccaggagatgaggaagcaaaaaatcaagtcaattctgaatcagattcagattctgaagaatctaagaagccaagatacagacataggcttttgcggcacaaattgactgtgagtgacggagaatctggagaagaaaaaaagacaaagcctaaagagcataaagaagtcaaaggcagaaacagaagaaaggtgagcagtgaagattcagaagattctgattttcaggaatcaggagttagtgaagaagttagtgaatccgaagatgaacagcggcccagaacaaggtctgcaaagaaagcagagttggaagaaaatcagcggagctataaacagaaaaagaaaaggcgacgtattaaggttcaagaagattcatccagtgaaaacaagagtaattctgaggaagaagaggaggaaaaagaagaggaggaggaagaggaggaggaggaggaagaggaggaggaagatgaaaatgatgattccaagtctcctggaaaaggcagaaagaaaattcggaagattcttaaagatgataaactgagaacagaaacacaaaatgctcttaaggaagaggaagagagacgaaaacgtattgctgagagggagcgtgagcgagaaaaattgagagaggtgatagaaattgaagatgcttcacccaccaagtgtccaataacaaccaagttggttttagatgaagatgaagaaaccaaagaacctttagtgcaggttcatagaaatatggttatcaaattgaaaccccatcaagtagatggtgttcagtttatgtgggattgctgctgtgagtctgtgaaaaaaacaaagaaatctccaggttcaggatgcattcttgcccactgtatgggccttggtaagactttacaggtggtaagttttcttcatacagttcttttgtgtgacaaactggatttcagcacggcgttagtggtttgtcctcttaatactgctttgaattggatgaatgaatttgagaagtggcaagagggattaaaagatgatgagaagcttgaggtttctgaattagcaactgtgaaacgtcctcaggagagaagctacatgctgcagaggtggcaagaagatggtggtgttatgatcataggctatgagatgtatagaaatcttgctcaaggaaggaatgtgaagagtcggaaacttaaagaaatatttaacaaagctttggttgatccaggccctgattttgttgtttgtgatgaaggccatattctaaaaaatgaagcatctgctgtttctaaagctatgaattctatacgatcaaggaggaggattattttaacaggaacaccacttcaaaataacctaattgagtatcattgtatggttaattttatcaaggaaaatttacttggatccattaaggagttcaggaatagatttataaatccaattcaaaatggtcagtgtgcagattctaccatggtagatgtcagagtgatgaaaaaacgtgctcacattctctatgagatgttagctggatgtgttcagaggaaagattatacagcattaacaaaattcttgcctccaaaacacgaatatgtgttagctgtgagaatgacttctattcagtgcaagctctatcagtactacttagatcacttaacaggtgtgggcaataatagtgaaggtggaagaggaaaggcaggtgcaaagcttttccaagattttcagatgttaagtagaatatggactcatccttggtgtttgcagctagactacattagcaaagaaaataagggttattttgatgaagacagtatggatgaatttatagcctcagattctgatgaaacctccatgagtttaagctccgatgattatacaaaaaagaagaaaaaagggaaaaaggggaaaaaagatagtagctcaagtggaagtggcagtgacaatgatgttgaagtgattaaggtctggaattcaagatctcggggaggtggtgaaggaaatgtggatgaaacaggaaacaatccttctgtttctttaaaactggaagaaagtaaagctacttcttcttctaatccaagcagcccagctccagactggtacaaagattttgttacagatgctgatgctgaggttttagagcattctgggaaaatggtacttctctttgaaattcttcgaatggcagaggaaattggggataaagtccttgttttcagccagtccctcatatctctggacttgattgaagattttcttgaattagctagtagggagaagacagaagataaagataaaccccttatttataaaggtgaggggaagtggcttcgaaacattgactattaccgtttagatggttccactactgcacagtcaaggaagaagtgggctgaagaatttaatgatgaaactaatgtgagaggacgattatttatcatttctactaaagcaggatctctaggaattaatctggtagctgctaatcgagtaattatattcgacgcttcttggaatccatcttatgacatccagagtatattcagagtttatcgctttggacaaactaagcctgtttatgtatataggttcttagctcagggaaccatggaagataagatttatgatcggcaagtaactaagcagtcactgtcttttcgagttgttgatcagcagcaggtggagcgtcattttactatgaatgagcttactgaactttatacttttgagccagacttattagatgaccctaattcagaaaagaagaagaagagggatactcccatgctgccaaaggataccatacttgcagagctccttcagatacataaagaacacattgtaggataccatgaacatgattctcttttggaccacaaagaagaagaagagttgactgaagaagaaagaaaagcagcttgggctgagtatgaagcagagaagaagggactgaccatgcgtttcaacataccaactgggaccaatttaccccctgtcagtttcaactctcaaactccttatattcctttcaatttgggagccctgtcagcaatgagtaatcaacagctggaggacctcattaatcaaggaagagaaaaagttgtagaagcaacaaacagtgtgacagcagtgaggattcaacctcttgaggatataatttcagctgtatggaaggagaacatgaatctctcagaggcccaagtacaggcgttagcattaagtagacaagccagccaggagcttgatgttaaacgaagagaagcaatctacaatgatgtattgacaaaacaacagatgttaatcagctgtgttcagcgaatacttatgaacagaaggctccagcagcagtacaatcagcagcaacagcaacaaatgacttatcaacaagcaacactgggtcacctcatgatgccaaagcccccaaatttgatcatgaatccttctaactaccagcagattgatatgagaggaatgtatcagccagtggctggtggtatgcagccaccaccattacagcgtgcaccacccccaatgagaagcaaaaatccaggaccttcccaagggaaatcaatgtgattttgcactaaaagcttaatggattgttaaaatcatagaaagatcttttatttttttaggaatcaatgacttaacagaactcaactgtataaatagtttggtccccttaaatgccaatcttccatattagttttactttttttttttttaaatagggcataccatttcttcctgacatttgtcagtgatgttgcctagaatcttcttacacacgctgagtacagaagatatttcaaattgttttcagtgaaaacaagtccttccataatagtaacaactccacagatttcctctctaaatttttatgcctgcttttagcaaccataaaattgtcataaaattaataaatttaggaaagaataaagatttatatattcattctttacatataaaaacacacagctgagttcttagagttgattcctcaagttatgaaatacttttgtacttaatccatttcttgattaaagtgattgaaatggttttaatgttcttttgactgaagtctgaaactgggctcctgctttattgtctctgtgactgaaagttagaaactgagggttatctttgacacagaattgtgtgcaatattcttaaatactactgctctaaaagttggagaagtcttgcagttatcttagcattgtataaacagccttaagtatagcctaagaagagaattcctttttcttctttagtccttctgccattttttattttcagttatatgtgctgaaataattactggtaaaatttcagggttgtggattatcttccacacatgaattttctctctcctggcacgaatataaagcacatctcttaactgcatggtgccagtgctaatgcttcatcctgttgctggcagtgggatgtggacttagaaaatcaagttctagcattttagtaggttaacactgaagttgtggttgttaggttcacaccctgttttataaacaacatcaaaatggcagaaccattgctgactttaggttcacatgaggaatgtacttttaacaattcccagtactatcagtattgtgaaataattcctctgaaagataagaatcactggcttctatgcgcttcttttctctcatcatcatgttcttttaccccagtttccttacatttttttaaattgtttcagagtttgttttttttttagtttagattgtgaggcaattattaaatcaaaattaattcatccaatacccctttactagaagttttactagaaaatgtattacattttattttttcttaatccagttctgcaaaaatgacctataaatttattcatgtacaattttggttacttgaattgttaaagaaaacattgtttttgactatgggagtcaactcaacatggcagaaccatttttgagatgatgatacaacaggtagtgaaacagcttaagaattccaaaaaaaaaaaaaaaaaaaaaaaaaagaaaactgggtttgggctttgctttaggtatcactggattagaatgagtttaacattagctaaaactgctttgagttgtttggatgattaagagattgccatttttatcttggaagaactagtggtaaaacatccaagagcactaggattgtgatacagaatttgtgaggtttggtggatccacgcccctctcccccactttcccatgatgaaatatcactaataaatcctgtatatttagatattatgctagccatgtaatcagatttatttaattgggtggggcaggtgtgtatttactttagaaaaaatgaaaaagacaagatttatgagaaatatttgaaggcagtacactctggccaactgttaccagttggtatttctacaagttcagaatattttaaacctgatttactagacctgggaattttcaacatggtctaattatttactcaaagacatagatgtgaaaattttaggcaaccttctaaatctttttcaccatggatgaaactataacttaaagaataatacttagaagggttaattggaaatcagagtttgaaataaaacttggaccactttgtatacactcttctcacttgacattttagctatataatatgtactttgagtataacatcaagctttaacaaatatttaaagacaaaaaaatcacgtcagtaaaatactaaaaggctcatttttatatttgttttagatgttttaaatagttgcaatggattaaaaatgatgatttaaaatgttgcttgtaatacagttttgcctgctaaattctccacattttgtaacctgttttatttctttgggtgtaaagcgtttttgcttagtattgtgatattgtatatgttttgtcccagttgtatagtaatgtttcagtccatcatccagctttggctgctgaaatcatacagctgtgaagacttgcctttgtttctgttagactgcttttcagttctgtattgagtatcttaagtactgtagaaaagatgtcacttcttcctttaaggctgttttgtaatatatataaggactggaattgtgtttttaaagaaaagcattcaagtatgacaatatactatctgtgttttcaccattcaaagtgctgtttagtagttgaaacttaaactatttaatgtcatttaataaagtgaccaaaatgtgttgtgctctttattgtattttcacagctttgaaaatctgtgcacatactgtttcatagaaaatgtatagcttttgttgtcctatataatggtggttcttttgcacatttagttatttaatattgagaggtcacgaagtttggttattgaatctgttatatactaaattctgtaaagggagatctctcatctcaaaaagaatttacataccaggaagtccatgtgtgtttgtgttagttttggatgtctttgtgtaatccagccccatttcctgtttcccaacagctgtaacactcattttaagtcaagcagggctaccaacccacacttgatagaaaagctgcttaccattcagaagcttccttattacctggcctccaaatgagctgaatattttgtagccttcccttagctatgttcattttccctccattatcataaaatcagatcgatatttatgtgccccaaacaaaactttaagagcagttacattctgtcccagtagcccttgtttcctttgagagtagcatgttgtgaggctatagagacttattctaccagtaaaacaggtcaatccttttacatgtttattatactaaaaattatgttcagggtatttactactttatttcaccagactcagtctcaagtgacttggctatctccaaatcagatctacccttagagaataaacatttttctaccgttattttttttcaagtctataatctgagccagtcccaaaggagtgatcaagtttcagaaatgctttcatcttcacaacattttatatatactattatatggggtgaataaagttttaaatccgaaatataaaaaaaaaaaaaaaaaaa

SEQ ID NO: 9 - protein, 2285 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQNYKRPMDGTYGPPAKRHEGEMYSVPYSTGQGQPQQQQLPPAQPQPASQQQAAQPSPQQDVYNQYGNAYPATATAATERRPAGGPQNQFPFQFGRDRVSAPPGTNAQQNMPPQMMGGPIQASAEVAQQGTMWQGRNDMTYNYANRQSTGSAPQGPAYHGVNRTDEMLHTDQRANHEGSWPSHGTRQPPYGPSAPVPPMTRPPPSNYQPPPSMQNHIPQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 10 - DNA, 8585 bp, Homo sapiens
cagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcagaattacaagcggccaatggatggcacatatggccctcctgccaagcggcacgaaggggagatgtacagcgtgccatacagcactgggcaggggcagcctcagcagcagcagttgcccccagcccagccccagcctgccagccagcaacaagctgcccagccttcccctcagcaagatgtatacaaccagtatggcaatgcctatcctgccactgccacagctgctactgagcgccgaccagcaggcggcccccagaaccaatttccattccagtttggccgagaccgtgtctctgcaccccctggcaccaatgcccagcaaaacatgccaccacaaatgatgggcggccccatacaggcatcagctgaggttgctcagcaaggcaccatgtggcaggggcgtaatgacatgacctataattatgccaacaggcagagcacgggctctgccccccagggccccgcctatcatggcgtgaaccgaacagatgaaatgctgcacacagatcagagggccaaccacgaaggctcgtggccttcccatggcacacgccagcccccatatggtccctctgcccctgtgccccccatgacaaggccccctccatctaactaccagcccccaccaagcatgcagaatcacattcctcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaagacttt

SEQ ID NO: 11 - protein, 2068 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 12 - DNA, 7934 bp, Homo sapiens
cagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaagacttt

(19) United States
(12) Patent Application Publication
(10) Pub. No.: US 2019/0070188 A1
(43) Pub. Date: Mar. 7, 2019

(54) PEDIATRIC DOSING FOR TREATMENT OF CANCER WITH AN EZH2 INHIBITOR

(71) Applicant: Epizyme, Inc., Cambridge, MA (US)

(72) Inventors: Heike KEILHACK, Belmont, MA (US); Sarah K. KNUTSON, Lincoln, MA (US); Nigel WATERS, Belmont, MA (US)

(21) Appl. No.: 15/773,757

(22) PCT Filed: Nov. 7, 2016
(86) PCT No.: PCT/US2016/060852

§ 371 Date: May 4, 2018

Related U.S. Application Data
(60) Provisional application No. 62/252,190, filed on Nov. 6, 2015.

(51) Int. Cl. A61K 31/5377; A61K 9/00; A61P 35/00

(52) U.S. Cl. CPC A61K 31/5377; A61K 9/0085; A61P 35/00; A61K 9/0053; A61K 9/0043

(57) ABSTRACT

The disclosure provides a method of treating a an INI1-deficient tumor in a subject in need thereof comprising administering to the subject a therapeutically-effective amount of an enhancer of a zeste homolog 2 (EZH2) inhibitor. In a preferred embodiment of this method, the subject is pediatric and the EZH2 inhibitor is Tazemetostat.

### RELATED APPLICATIONS

This application claims priority to, and the benefit of U.S. Provisional Application No. 62/252,190 filed Nov. 6, 2015 the content of which is incorporated herein by reference in its entirety.

### INCORPORATION-BY-REFERENCE OF SEQUENCE LISTING

The contents of the text file named “EPIZ058NO1US_ST25.txt,” which was created on Sep. 25, 2018 and is 140 KB in size, are hereby incorporated by reference in their entireties.

### FIELD OF THE DISCLOSURE

The disclosure is directed to the fields of small molecule therapies, cancer, and methods of treating rare cancer types, particularly in pediatric subjects.

### BACKGROUND

There is a long-felt yet unmet need for effective treatments for certain cancers caused by genetic alterations or loss of function of subunits of the SWI/SNF chromatin remodeling complex that result in EZH2-dependent oncogenesis.

### SUMMARY

Some aspects of this disclosure provide methods, strategies, and dosage schedules for inhibiting EZH2 in a subject, e.g., in a human pediatric patient, by administering a therapeutically-effective amount of an enhancer of a zeste homolog 2 (EZH2) inhibitor to the subject. The methods, strategies, and dosage schedules provided herein are useful, for example, for treating cancer in pediatric patients.

Some aspects of this disclosure provide a method of treating a cancer, e.g., an INI1-deficient tumor, in a subject in need thereof comprising administering to the subject a therapeutically-effective amount of an enhancer of a zeste homolog 2 (EZH2) inhibitor. Methods of treating cancer, e.g., INI1-deficient tumors, provided herein may comprise preventing and/or inhibiting proliferation of a malignant cell, e.g., an INI1-deficient cell, or cell population.

In certain embodiments of the methods of the disclosure, the EZH2 inhibitor comprises

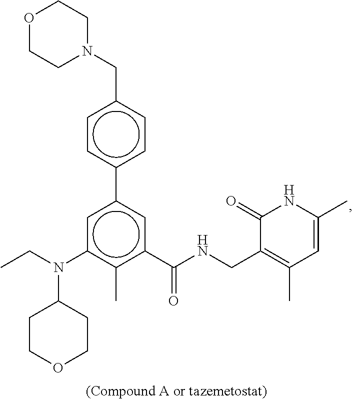

or a pharmaceutically-acceptable salt thereof.

In certain embodiments of the methods of the disclosure, the EZH2 inhibitor comprises

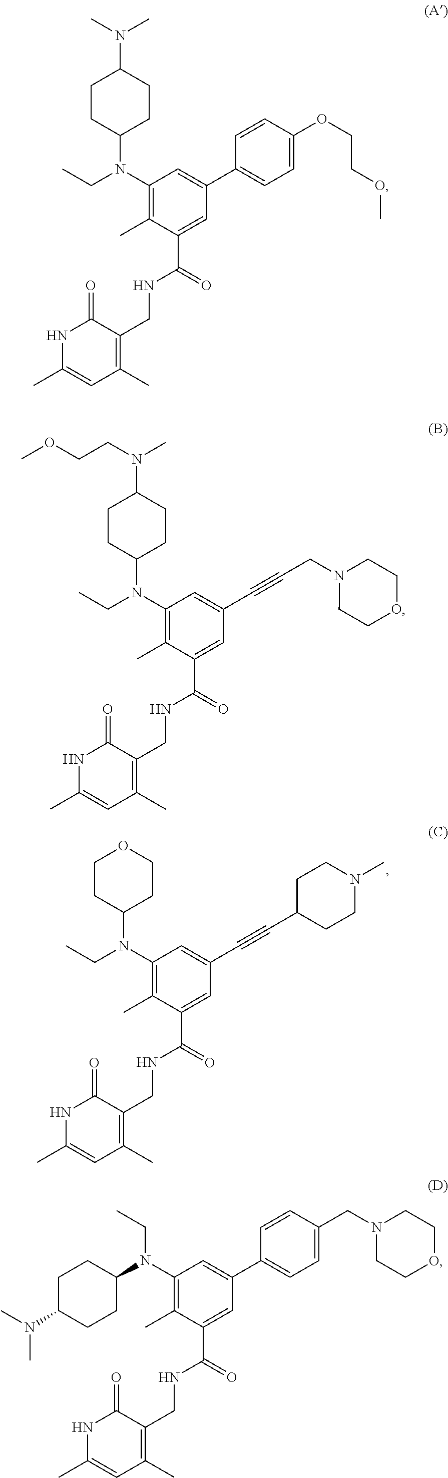

a stereoisomer, a pharmaceutically acceptable salt and/or a solvate thereof.

In certain embodiments of the methods of the disclosure, the EZH2 inhibitor comprises

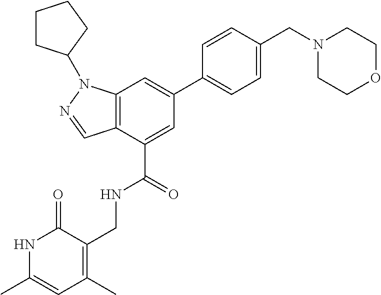 [SMILES: Cc1cc(C)c(CNC(=O)c2cc(-c3ccc(CN4CCOCC4)cc3)cc3c2cnn3C2CCCC2)c(=O)[nH]1]

or a pharmaceutically acceptable salt thereof.

In certain embodiments of the methods of the disclosure, the EZH2 inhibitor comprises

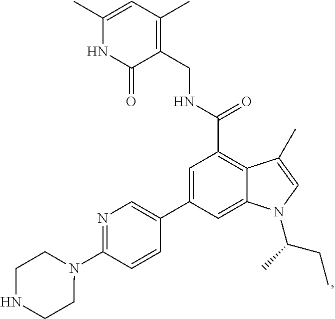 [SMILES: CC[C@H](C)n1cc(C)c2c(C(=O)NCc3c(C)cc(C)[nH]c3=O)cc(-c3ccc(N4CCNCC4)nc3)cc21]

a stereoisomer, a pharmaceutically acceptable salt and/or a solvate thereof

In certain embodiments of the methods of the disclosure, the EZH2 inhibitor comprises

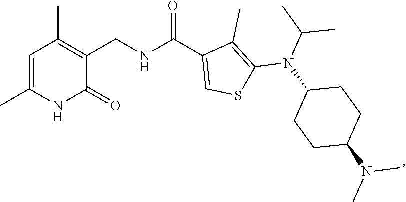

a stereoisomer, a pharmaceutically acceptable salt and/or a solvate thereof.

In certain embodiments of the methods of the disclosure, the EZH2 inhibitor comprises

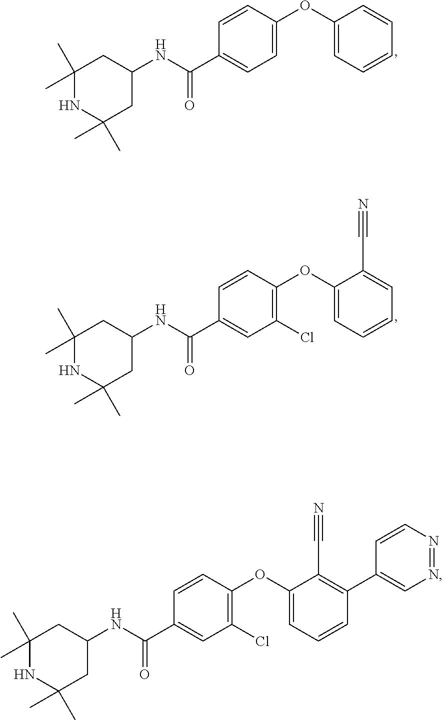 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

a stereoisomer, a pharmaceutically acceptable salt and/or a solvate thereof.

EZH2 inhibitors of the disclosure may be administered orally. For example, the EZH2 inhibitor may be formulated as an oral tablet or suspension.

EZH2 inhibitors of the disclosure may be formulated for administration to cerebral spinal fluid (CSF) by any route. Exemplary routes of administration to the CSF include, but are not limited to, an intraspinal, an intracranial, an intrathecal or an intranasal route.

In certain embodiments, including, but not limited to, those embodiments wherein the EZH2 inhibitor is formulated as an oral tablet, or as a suspension or solution, EZH2 inhibitors of the disclosure may be administered at a dose of between 10 mg/kg/day and 1600 mg/kg/day. EZH2 inhibitors of the disclosure may be administered at a dose of about 100, 200, 400, 800, or 1600 mg. EZH2 inhibitors of the disclosure may be administered at a dose of about 800 mg. EZH2 inhibitors of the disclosure may be administered once or twice per day (BID). In some embodiments, EZH2 inhibitors of the disclosure may be administered at a dose of between 10 mg/kg/day and 1600 mg/kg/day BID. For example, in some embodiments, EZH2 inhibitors of the disclosure may be administered at a dose of 800 mg BID.

In some embodiments, including, but not limited to, those embodiments wherein the EZH2 inhibitor is formulated as an oral tablet, suspension, or solution, and/or formulated for administration to the CSF by any route, the EZH2 inhibitor may be administered at a dose of between 10 mg/kg/day and 1600 mg/kg/day, e.g., at a dose of 10 mg/kg/day, 20 mg/kg/day, 25 mg/kg/day, 30 mg/kg/day, 40 mg/kg/day, 50 mg/kg/day, 60 mg/kg/day, 70 mg/kg/day, 75 mg/kg/day, 80 mg/kg/day, 90 mg/kg/day, 100 mg/kg/day, 200 mg/kg/day, 250 mg/kg/day, 300 mg/kg/day, 400 mg/kg/day, 500 mg/kg/day, 600 mg/kg/day, 700 mg/kg/day, 750 mg/kg/day, 800 mg/kg/day, 900 mg/kg/day, 1000 mg/kg/day, 1100 mg/kg/day, 1200 mg/kg/day, 1250 mg/kg/day, 1300 mg/kg/day, 1400 mg/kg/day, 1500 mg/kg/day, or 1600 mg/kg/day. For example, EZH2 inhibitors of the disclosure may be administered at a dose of between 10 mg/kg/day and 1600 mg/kg/day BID. For example, EZH2 inhibitors of the disclosure may be administered at a dose of 800 mg BID.

In some embodiments, including, but not limited to, those embodiments wherein the EZH2 inhibitor is formulated as an oral tablet, suspension, or solution, and/or formulated for administration to the CSF by any route, the EZH2 inhibitor may be administered at a dose of between 10 mg/m²/day and 1200 mg/m²/day, e.g., at a dose of 10 mg/m²/day, 20 mg/m²/day, 25 mg/m²/day, 30 mg/m²/day, 40 mg/m²/day, 50 mg/m²/day, 60 mg/m²/day, 70 mg/m²/day, 75 mg/m²/day, 80 mg/m²/day, 90 mg/m²/day, 100 mg/m²/day, 110 mg/m²/day, 120 mg/m²/day, 125 mg/m²/day, 130 mg/m²/day, 140 mg/m²/day, 150 mg/m²/day, 160 mg/m²/day, 170 mg/m²/day, 175 mg/m²/day, 180 mg/m²/day, 190 mg/m²/day, 200 mg/m²/day, 210 mg/m²/day, 220 mg/m²/day, 225 mg/m²/day, 230 mg/m²/day, 240 mg/m²/day, 250 mg/m²/day, 260 mg/m²/day, 270 mg/m²/day, 275 mg/m²/day, 280 mg/m²/day, 290 mg/m²/day, 300 mg/m²/day, 310 mg/m²/day, 320 mg/m²/day, 325 mg/m²/day, 330 mg/m²/day, 340 mg/m²/day, 350 mg/m²/day, 360 mg/m²/day, 370 mg/m²/day, 375 mg/m²/day, 380 mg/m²/day, 390 mg/m²/day, 400 mg/m²/day, 410 mg/m²/day, 420 mg/m²/day, 425 mg/m²/day, 430 mg/m²/day, 440 mg/m²/day, 450 mg/m²/day, 460 mg/m²/day, 470 mg/m²/day, 475 mg/m²/day, 480 mg/m²/day, 490 mg/m²/day, 500 mg/m²/day, 525 mg/m²/day, 550 mg/m²/day, 575 mg/m²/day, 600 mg/m²/day, 625 mg/m²/day, 650 mg/m²/day, 675 mg/m²/day, 700 mg/m²/day, 750 mg/m²/day, 800 mg/m²/day, 850 mg/m²/day, 900 mg/m²/day, or 1000 mg/m2/day. In some embodiments, including, but not limited to, those embodiments wherein the EZH2 inhibitor is formulated as an oral tablet, suspension, or solution, and/or formulated for administration to the CSF by any route, the EZH2 inhibitor may be administered at a dose of between 10 mg/m²/day and 1200 mg/m²/day, e.g., between 100 and 300 mg/m²/day, between 200 and 300 mg/m²/day, between 200 and 400 mg/m²/day, between 250 and 500 mg/m²/day, between 150 and 400 mg/m²/day, between 150 and 300 mg/m²/day, between 300 and 600 mg/m²/day, between 350 and 400 mg/m²/day, between 350 and 700 mg/m²/day, or between 400 and 1200 mg/m²/day. For example, EZH2 inhibitors of the disclosure may be administered at a dose of between 10 mg/m²/day and 1200 mg/m²/day BID. For example, EZH2 inhibitors of the disclosure may be administered at a dose of 100, 120, 140, 150, 160, 200, 240, 250, 260, 300, 320, 350, 380, 400, or 600 mg/m2 BID.

In certain embodiments, including, but not limited to, those embodiments wherein the EZH2 inhibitor is formulated as an oral tablet, or as a suspension or solution and/or formulated for administration to the CSF by any route, EZH2 inhibitors of the disclosure may be administered at a dose of 50%, 60%, 70%, 80%, 90%, or any percentage in between of a value of an area under the curve (AUC) of a steady state plasma and/or CSF concentration (AUCss) of an EZH2 inhibitor, wherein the AUCss is determined following administration of the EZH2 inhibitor to an adult subject at a dose of between 10 mg/kg/day and 1600 mg/kg/day BID. In certain embodiments of the methods of the disclosure, including, but not limited to, those embodiments wherein the EZH2 inhibitor is formulated as an oral suspension and/or formulated to administration to the CSF by any route, EZH2 inhibitors of the disclosure may be administered at a dose of between 230 mg/m² and 600 mg/m², inclusive of the endpoints. EZH2 inhibitors of the disclosure may be administered at a dose of between 300 mg/m² and 600 mg/m². EZH2 inhibitors of the disclosure may be administered at a dose of between 230 mg/m² and 305 mg/m², inclusive of the endpoints. EZH2 inhibitors of the disclosure may be administered at a dose of 240 mg/m². EZH2 inhibitors of the disclosure may be administered at a dose of 300 mg/m². EZH2 inhibitors of the disclosure may be administered once or twice per day (BID). For example, EZH2 inhibitors of the disclosure may be administered at a dose of between 230 mg/m² and 600 mg/m² BID, inclusive of the endpoints.

For example, an EZH2 inhibitor of the disclosure may be administered at a dose of about 60% of the area under the curve (AUC) at steady state (AUCss) following administration of 1600 mg twice a day to an adult subject. Accordingly, an EZH2 inhibitor of the disclosure administered at a dose of about 60% of the area under the curve (AUC) at steady state (AUCss) following administration of 1600 mg twice a day to an adult subject, is administered at a dose of about 600 mg/m² per day or at least 600 mg/m² per day. In certain aspects of this example, the subject treated with the EZH2 inhibitor is a pediatric subject.

For example, an EZH2 inhibitor of the disclosure may be administered at a dose of about 80% of the area under the curve (AUC) at steady state (AUCs) following administration of 800 mg twice a day to an adult subject. Accordingly, an EZH2 inhibitor of the disclosure administered at a dose of about 80% of the area under the curve (AUC) at steady state (AUCss) following administration of 800 mg twice a day to an adult subject, is administered at a dose of about 390 mg/m² per day or at least 390 mg/m² per day. In certain aspects of this example, the subject treated with the EZH2 inhibitor is a pediatric subject.

In some embodiment, the subject may be a pediatric subject. In some embodiments, a pediatric subject of the disclosure is between 6 months and 21 years of age, inclusive of the endpoints. For example, in some embodiments, a pediatric subject of the disclosure is between 1 year and 18 years of age, inclusive of the endpoints; 10 years of age or less; 5 years of age or less; between 6 months and 1 year of age, inclusive of the endpoints; between 1 year and 2 years of age, inclusive of the endpoints; between 2 years and 6 years of age, inclusive of the endpoints; between 6 years and 12 years of age, inclusive of the endpoints; or between 12 years and 18 years of age, inclusive of the endpoints. In some embodiments, a pediatric subject is about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, or about 21 years of age. In some embodiments, a pediatric subject is at least 1, 2, 3, 4, 5, 6, 7, 8, 9, 10, or 12 years of age, and not more than 5, 6, 7, 8, 9, 10, 11, 12, 13, 14 ,15, 16, 17, 18, 19, 20, or 21 years of age, wherein every possible age range that can be formed with these values (e.g., at least 4 and not older than 12 years, or at least 10 and not older than 18 years, to provide two non-limiting examples) is embraced by the present disclosure.

In some embodiments, the disclosure provides a method of treating cancer, e.g., an INI1-deficient tumor, in a subject in need thereof comprising administering to the subject a therapeutically-effective amount of tazemetostat, wherein the therapeutically effective amount is at least 300 mg/m² twice per day (BID), and wherein the subject is a pediatric subject, e.g., a subject between 6 months and 21 years of age, inclusive of the endpoints.

### BRIEF DESCRIPTION OF THE DRAWINGS

FIGS. 1A and 1B are a series of Western blot analyses of cell lines with wild type (RD and SJCRH30) and mutant SNFS.

FIGS. 2A-2E are a series of graphs establishing that SNFS mutant cell lines A204 (C), G401 (D) and G402 (E) selectively respond to EZH2 compound (Compound D) compared to wild type cell lines RD (A) and SJCRH30 (B).

FIGS. 3A-3D are a series of bar graphs showing that G401 SNF mutant cell line is responding to Compound D after 7 days in soft agar compared to wild type cells RD. A shows cell line RD (5,000 cells/well). B shows G401 cells (5,000 cells/well). C shows G401 cells in 2D growth. D shows G401 cells (10,000 cells/well).

FIGS. 4A-4D are four graphs showing that G401 SNFS mutant cell line is sensitive to Compound A in vitro. Wild type cell line SJCRH30 (A) and RD (C) and SNFS mutant cell line G401 (B) and A204 (D) were pretreated for 7 days with indicated concentrations of Compound A and replated on day 0. Cell viability was determined by CellTiter-Glo® Luminescent Cell Viability Assay.

FIGS. 5A-5E are a series of graphs showing durable regressions in G401 xenografts (malignant rhabdoid tumor model) with Compound A treatment. (A) Tumor regressions induced by Compound A at the indicated doses. (B) Tumor regressions induced by twice daily administration of Compound A at the indicated doses. Data represent the mean values ±SEM (n=8). Compound administration was stopped on day 28. (C) EZH2 target inhibition in G401 xenograft tumor tissue collected from a parallel cohort of mice on day 21. Each point shows the ratio of H3K27Me3 to total H3. Horizontal lines represent group mean values. BLLQ=below lower limit of quantification. (D, E) Immunohistochemical staining of tumor histone methylation of tumor samples from the vehicle treated (D) and Compound A treated (E) (at 125 mg/kg) mice.

FIG. 6 is a graph showing the locations of ATRX mutations identified in SCLC cell lines.

FIG. 7A is a graph showing that LNCAP prostate cancer cells display dose-dependent cell growth inhibition with Compound D treatment in vitro.

FIG. 7B is a graph showing IC50 value of Compound D at day 11 and day 14 for WSU-DLCL2 and LNCAP cells.

FIGS. 8A-8C are three graphs establishing that ATRX mutant SCLC lines NCI-H446 (A), SW1271 (B) and NCI-H841 (C) are responding to Compound D.

FIGS. 9A-9C are three microscopy images showing that SCLC line NCI-H841 changes morphology after treatment with vehicle (A) or Compound D at concentration of 4.1E-02 uM (B) or 3.3 uM (C).

FIGS. 10A-10C are a series of graphs showing effects of Compound A on cellular global histone methylation and cell viability. (A) Chemical structure of Compound A (or tazemetostat). (B) Concentration-dependent inhibition of cellular H3K27Me3 levels in G401 and RD cells. (C) Selective inhibition of proliferation of SMARCB1-deleted G401 cells by Compound A in vitro (measured by ATP content). G401 (panels a and b) and RD cells (panels c and d) were re-plated at the original seeding densities on day 7. Each point represents the mean for each concentration (n=3).

FIGS. 11A and 11B are a series of graphs showing biochemical mechanism of action studies. The IC50 value of Compound A increases with increasing SAM concentration (A) and is minimally affected by increasing oligonucleosome concentration (B), indicating SAM-competitive and nucleosome-noncompetitive mechanism of action.

FIGS. 12A and 12B are a series of panels demonstrating verification of SMARCB1 and EZH2 expression in cell lines and specificity of Compound A for inhibition of cellular histone methylation. (A) Cell lysates were analyzed by immunoblot with antibodies specific to SMARCB1, EZH2 and Actin (loading control). (B) Selective inhibition of cellular H3K27 methylation in G401 and RD cells. Cells were incubated with Compound A for 4 days, and acid-extracted histones were analyzed by immunoblot.

FIGS. 13A and 13B are a series of bar graphs demonstrating that Compound A induces G₁ arrest and apoptosis in SMARCB1-deleted MRT cells. Cell cycle analysis (by flow cytometry) and determination of apoptosis (by TUNEL assay) in RD (panel A) or G401 cells (panel B) during incubation with either vehicle or 1 μM Compound A for up to 14 days. G₁ arrest was observed as of day 7 and apoptosis was induced as of day 11. Data are represented as mean values±SEM (n=2). The DMSO control values shown are the average±SEM from each time point. Cells were split and re-plated on days 4, 7 and 11 at the original seeding density.

FIGS. 14A-14C are a series of graphs showing that Compound A induces changes in expression of SMARCB1 regulated genes and cell morphology. (A) Basal expression of SMARCB1 regulated genes in G401 SMARCB1-deleted cells, relative to RD control cells (measured by qPCR, n=2). (B) G401 and RD cells were incubated with either DMSO or 1 μM Compound A for 2, 4 and 7 days. Gene expression was determined by qPCR (n=2) and is expressed relative to the DMSO control of each time point. Panels a-j correspond to genes GLI1, PTCh1, DOCK4, CD133, PTPRK, BIN1, CDKN1A, CDKN2A, EZH2, and MYC, respectively. (C) G402 cells were incubated with either DMSO (left panel) or 1 μM Compound A (right panel) for 14 days. Cells were split and re-plated to the original seeding density on day 7.

FIGS. 15A-15D are series of graphs demonstrating body weights, tumor regressions and plasma levels in G401 xenograft bearing mice treated with Compound A. (A) Body weights were determined twice a week for animals treated with Compound A on a BID schedule for 28 days. Data are presented as mean values ±SEM (n=16 until day 21, n=8 from day 22 to 60). (B) Tumor regressions induced by twice daily (BID) administration of Compound A for 21 days at the indicated doses (mean values ±SEM, n=16). *p<0.05, **p<0.01, repeated measures ANOVA, Dunnett's post-test vs. vehicle. (C) Tumor weights of 8 mice euthanized on day 21. ****p<0.0001, Fisher's exact test. (D) Plasma was collected 5 min before and 3 h after dosing of Compound A on day 21, and compound levels were measured by LC-MS/MS. Animals were euthanized, and tumors were collected 3 h after dosing on day 21. Tumor homogenates were generated and subjected to LC-MS/MS analysis to determine Compound A concentrations. Note that tumor compound levels could not be determined from all animals especially in the higher dose groups because the xenografts were too small on day 21. Dots represent values for the individual animals; horizontal lines represent group mean values.

FIGS. 16A-16C are a series of graphs showing that Compound A eradicates SMARCB1-deleted MRT xenografts in SCID mice. (A) Tumor regressions induced by twice daily (BID) administration of Compound A for 28 days at the indicated doses. Compound administration was stopped on day 28 and tumors were allowed to re-grow until they reached 2000 mm³ (data shown as mean values ±SEM, n=8). (B) EZH2 target inhibition in G401 xenograft tumor tissue collected from mice euthanized on day 21. Each point shows the ratio of H3K27Me3 to total H3, measured by ELISA. Horizontal lines represent group mean values; grey symbols are values outside of the ELISA standard curve. (C) Change in gene expression in G401 xenograft tumor tissue collected from mice treated with Compound A for 21 days. Panels a-d correspond to genes CD133, PTPRK, DOCK4, and GLI1, respectively. Data are presented as fold change compared to vehicle ±SEM (n=6, n=4 for 500 mg/kg group). *p<0.05, **p<0.01, ****p<0.0001, vs. vehicle, Fisher's exact test.

FIG. 17 is a schematic diagram depicting epigenetic control of gene expression. Combinations of histone modifications encode information that governs coordinated activation or repression of genetic programs as well as developmental cell identity and fate decisions.

FIG. 18 is a graph showing that EZH2 is over expressed and associated with chromosome 7 amplification in medulloblastoma. Solid bars indicate a balanced chromosome 7 whereas hatched bars indicate a chromosome 7 gain.

FIG. 19 is a schematic diagram depicting control of histone lysine methylation by EZH2 and MLL.

FIG. 20A is a graph showing the probability of overall survival (OS) as a function of time since diagnosis (in months) with medulloblastoma. Histone lysine methylation is altered in medulloblastoma. H3K27me3 abundance is increased in medulloblastoma cells compared to control cells.

FIG. 20B is a graph showing the probability of overall survival (OS) as a function of time since diagnosis (in months) with medulloblastoma. Histone lysine methylation is altered in medulloblastoma. H3K27me3 abundance is increased in medulloblastoma cells compared to control cells.

FIG. 21A is a series of photographs and a graph showing the abundances of H3K4me3 and H3K27Me3 in medulloblastoma cells. The data demonstrate deregulation of the histone code in medulloblastoma.

FIG. 21B is a graph depicting the probability of overall survival as a function of time since diagnosis (in months) for medulloblastoma subjects having deregulated histone methylation at H3K4me3 and/or H3K27Me3.

FIG. 22A is a graph demonstrating that inhibition of EZH2 by a short-hairpin EZH2 (shEZH2) construct suppresses medulloblastoma cell growth (growth of the DAOY medulloblastoma cell line) compared to a negative-control construct.

FIG. 22B a series of photographs and a graph demonstrating that inhibition of EZH2 by a short-hairpin EZH2 (shEZH2) construct suppresses medulloblastoma cell growth (growth of the DAOY medulloblastoma cell line) compared to a negative-control construct and/or the empty pSIF vector control.

FIG. 23A is a schematic diagram depicting the mechanism by which INI1 loss creates an oncogenic dependency on EZH2 in tumors.

FIG. 23B is a graph showing the percent of tumor-free survival of INI1 deficient mice as a function of time (days) when EZH2 is knocked out. EZH2 knockout reverses oncogenesis induced by INI1 loss.

FIG. 24A is a series of photographs showing control or EZH2 inhibitor-treated (DNZep-treated) atypical teratoid rhabdoid tumors (ATRTs) at 1, 3, 5, and 7 days post-treatment. Inhibition of EZH2 suppresses ATRT cell self-renewal.

FIG. 24B is a graph quantifying the results of FIG. 24A.

FIG. 24C is a graph quantifying the results of FIG. 24A.

FIG. 24D is a series of photographs showing control or EZH2 inhibitor-treated (DNZep-treated) atypical teratoid rhabdoid tumors (ATRTs) at 3, 5, 8 and 10 days post-treatment. Inhibition of EZH2 suppresses ATRT cell self-renewal.

FIG. 24E is a graph quantifying the results of FIG. 24D.

FIG. 25A is a pair of graphs showing a surviving fraction of untreated or DZNEP-treated ATRT cells (from a BT-16 ATRT cell line) exposed to 2Gy radiation. Inhibition of EZH2 radio-sensitizes ATRT.

FIG. 25B is a pair of graphs showing a surviving fraction of untreated or DZNEP-treated ATRT cells (from a UPN737 ATRT cell line, “737”) exposed to 2Gy radiation. Inhibition of EZH2 radio-sensitizes ATRT.

FIG. 26A is a graph showing the concentration of medulloblastoma cells (total cells per milliliter) as a function of time (days) following treatment with GSK-126, a small molecule inhibitor of EZH2. Small molecule inhibitors of EZH2 decrease medulloblastoma cell growth.

FIG. 26B is a graph showing the concentration of medulloblastoma cells (total cells per milliliter) as a function of time (days) following treatment with UNC 1999, a small molecule inhibitor of EZH2. Small molecule inhibitors of EZH2 decrease medulloblastoma cell growth.

FIG. 26C is a graph showing the concentration of medulloblastoma cells (total cells per milliliter) as a function of time (days) following treatment with tazemetostat (EPZ 6438), a small molecule inhibitor of EZH2. Small molecule inhibitors of EZH2 decrease medulloblastoma cell growth.

FIG. 26D is a graph showing the concentration of medulloblastoma cells (total cells per milliliter) as a function of time (days) following treatment with GSK-126, UNC 1999, and tazemetostat (EPZ 6438). Tazemetostat has the greatest effect on medulloblastoma cell growth of the small molecule inhibitors tested.

FIG. 27A is a chemical structure diagram of tazemetostat.

FIG. 27B is a pair of schematic diagrams depicting the relative selectivity of tazemetostat for EZH2.

FIG. 28A is a schematic diagram depicting the process by which primary medulloblastoma cell growth is evaluated ex vivo.

FIG. 28B is a graph depicting the relative abundances (percent of cells) of untreated or tazemetostat (EPZ 6438)-treated primary medulloblastoma cells in various cell cycle stages (sub Go/G1, Go/G1, S, or G2/M). A slice culture of medulloblastoma was freshly isolated from a 5 year old subject. The slice culture was treated with tazemetostat for 4 days before being disaggregated and analyzed by flow cytometry. Tazemetostat treatment decreases primary medulloblastoma cell growth ex vivo.

FIG. 28C is a graph depicting BrdU expression of the cells analyzed in FIG. 28B. Tazemetostat treatment decreases primary medulloblastoma cell growth ex vivo.

FIG. 29A is a graph depicting percent survival of vehicle or tazemetostat (EPZ 6438)-treated ATRT cells in vivo as a function of time (days) post-treatment. Tazemetostat decreases ATRT in vivo.

FIG. 29B is a photograph of a Western blot showing the relative amounts of H2K27me3 and H3 in vehicle or tazemetostat (EPZ 6438)-treated ATRT cells from FIG. 29A.

FIG. 30 is a schematic illustrating the generalized layout of a physiologically-based pharmacokinetic (PBPK) model.

FIG. 31 is a scheme illustrating the modeling and simulation for pediatric starting dose selection in early clinical development.

FIG. 32 is a series of graphs showing that the model fit for interim adult PK data at steady-state (Day 15, n=24) showed a good fit for each dose group. Symbols represent observed data from individuals (+/−SD, n=3 or 6 per dose) and the solid line represents the mean profile predicted from the PBPK model in Gastroplus™. The dotted grey lines represent the 90% CI. Tazemetostat PK data from patients enrolled in the dose escalation cohorts of a phase 1 clinical study were previously presented by Ribrag et al., Blood (2015) 126:473, the content of which is incorporated herein by reference in its entirety.

FIG. 33 is a pair of graphs showing the predicted mean total steady state plasma concentration-time profiles of tazemetostat administered as a 240 mg/m² BID or 300 mg/m² BID oral dose across the age ranges and mean measured total steady-state plasma concentration-time profile of tazemetostat administered as a 390 mg/m² (800mg) or 780 mg/m² (1600mg) BID oral dose in adults (n=6 per dose). The adult model was used to predict the PK profile in pediatric populations by accounting for age-dependent physiological differences, such as ontogeny of the GI tract and other organs, blood flows, P450 expression, plasma protein binding and hematocrit.

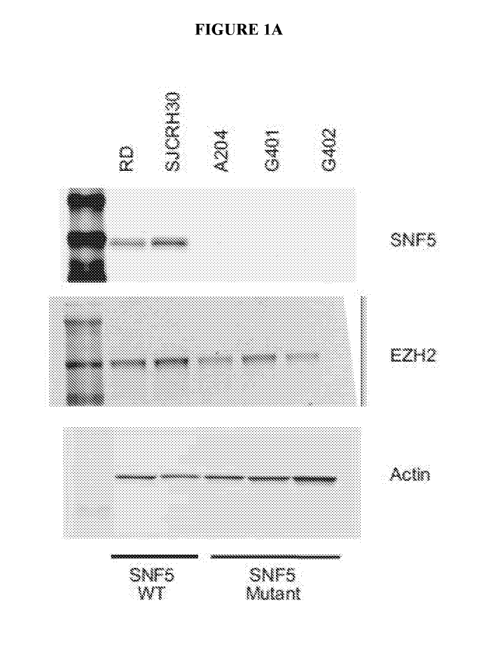

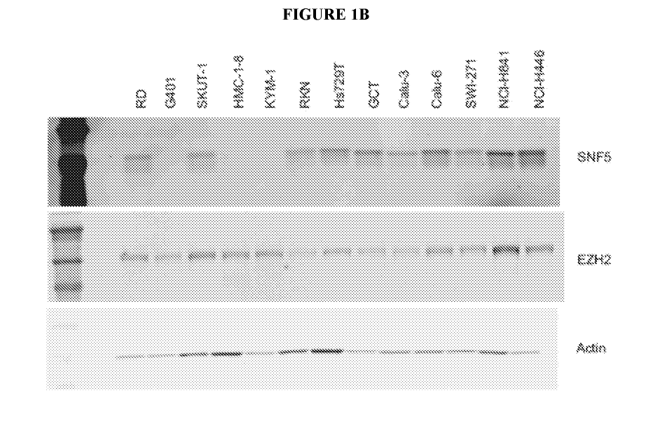

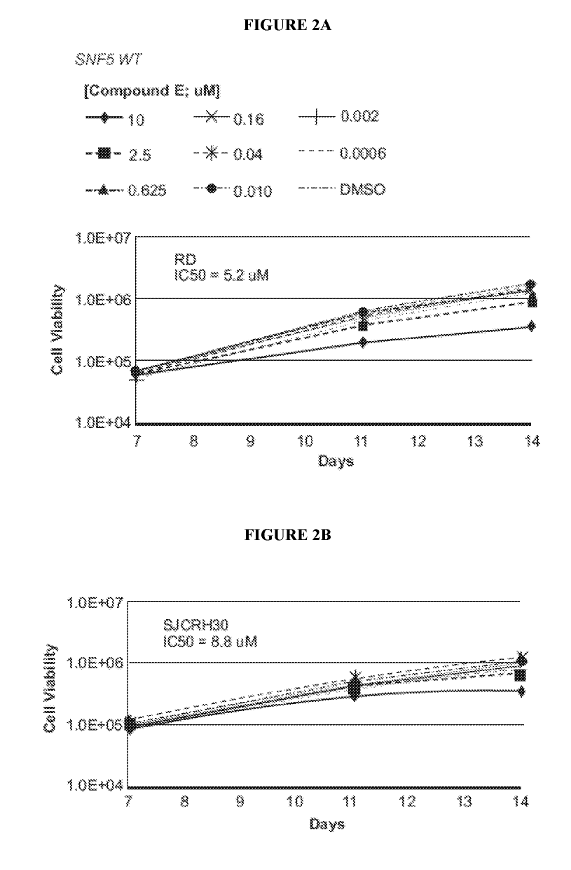

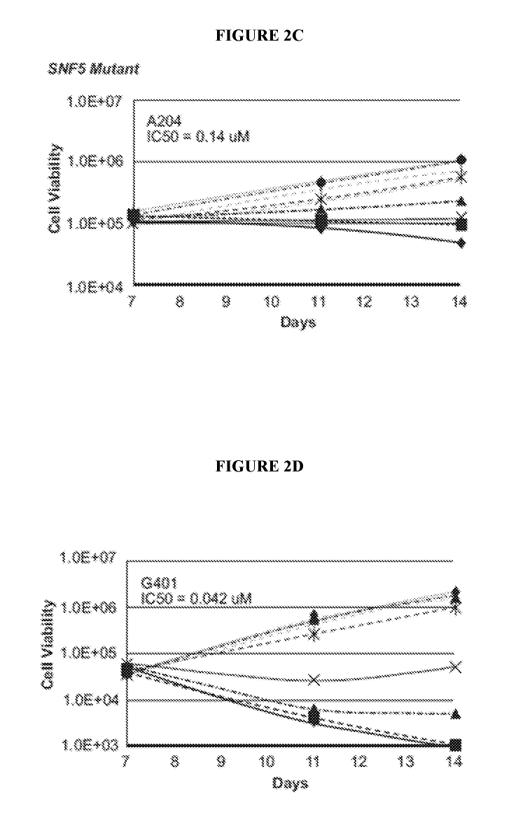

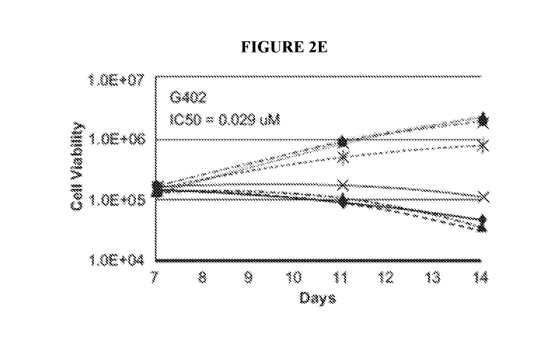

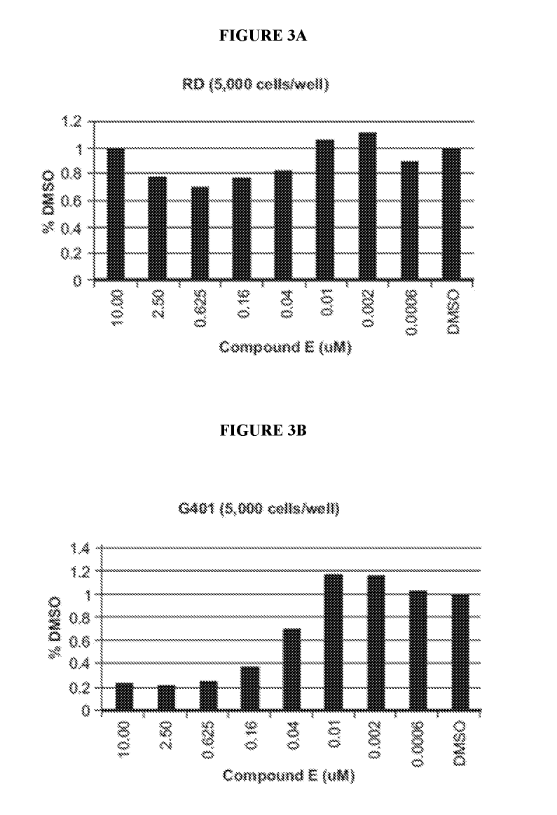

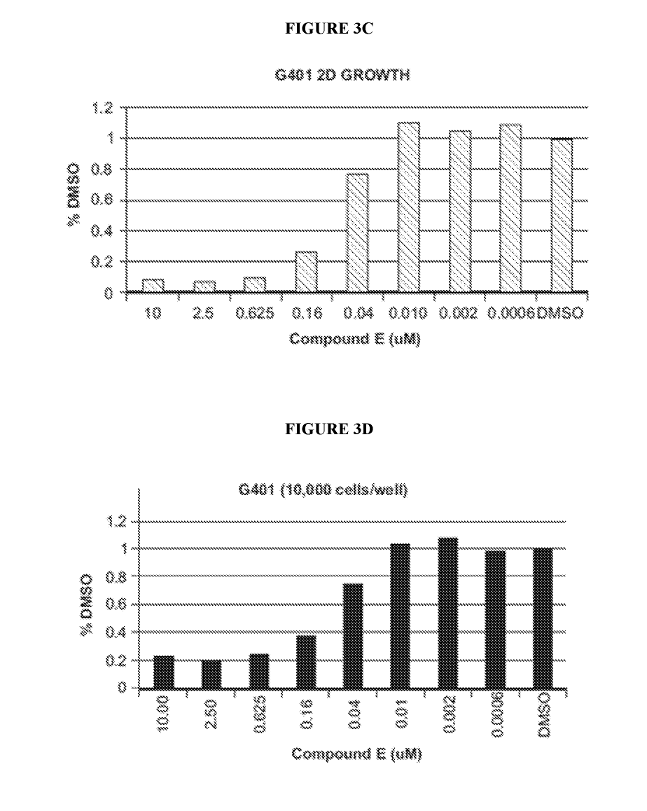

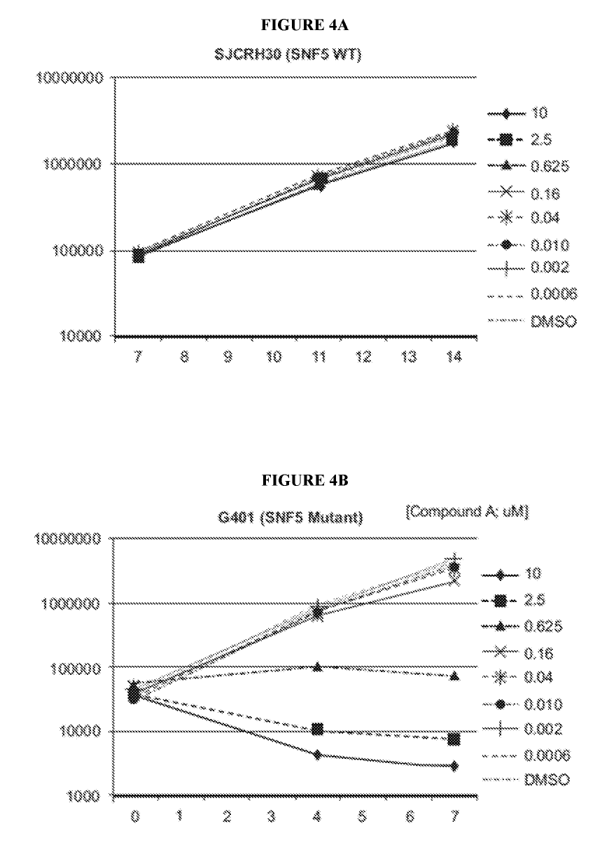

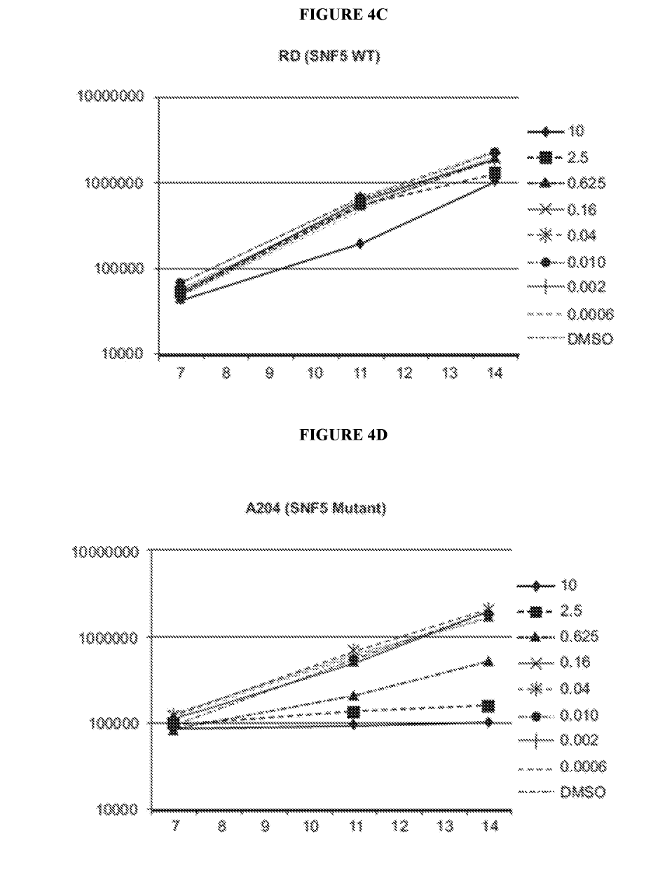

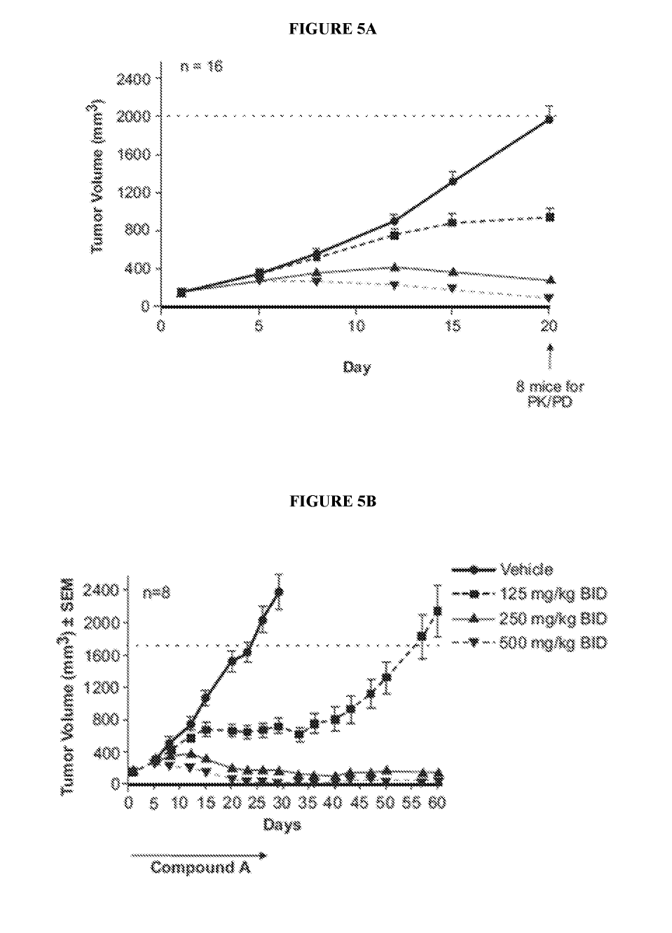

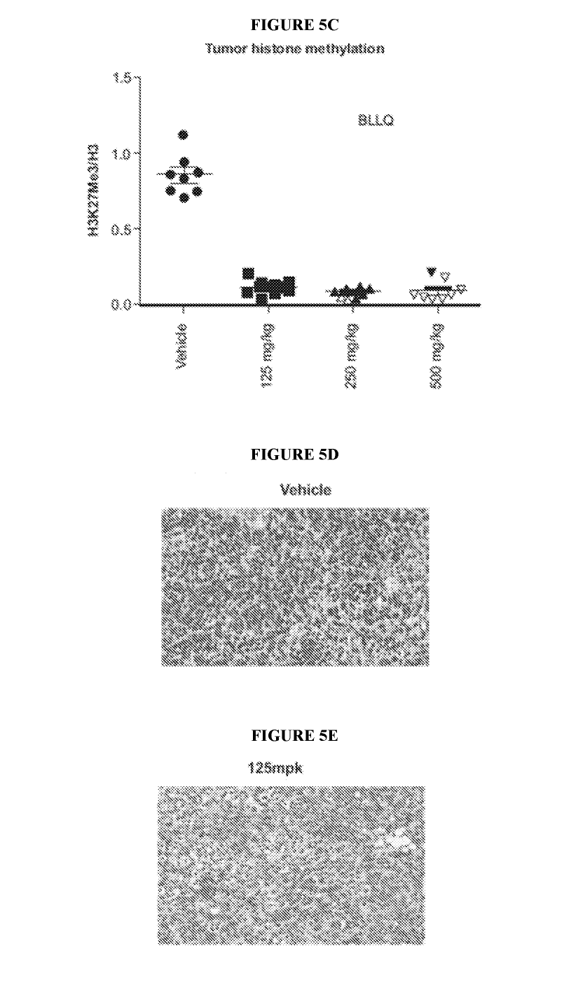

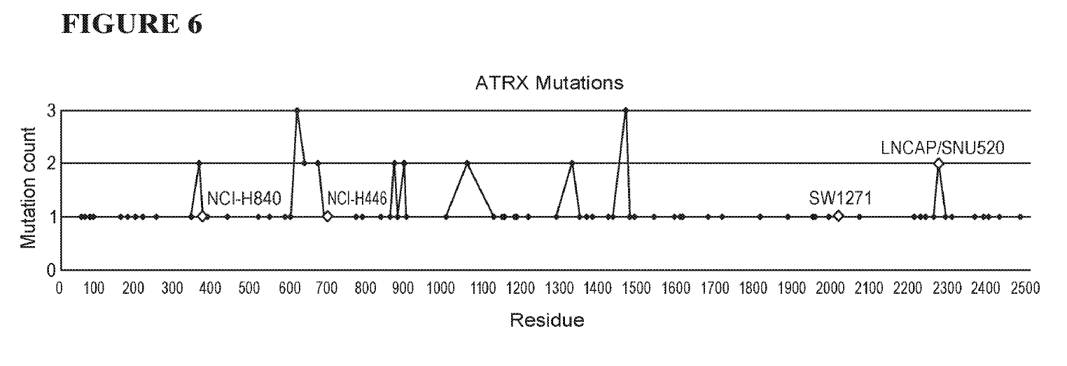

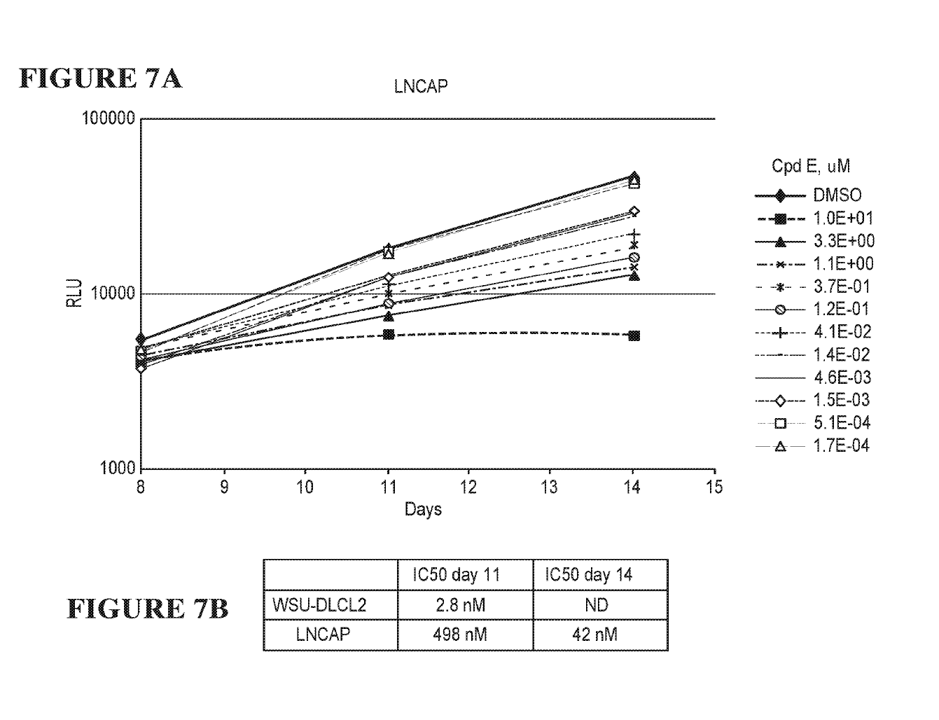

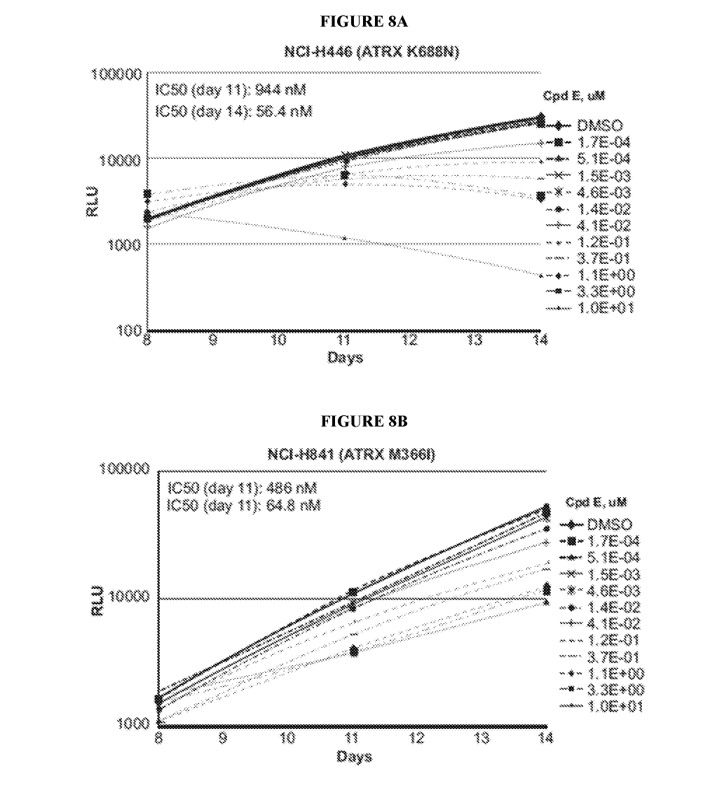

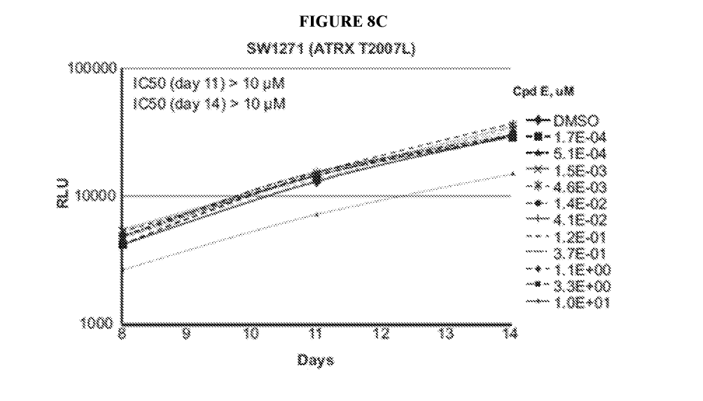

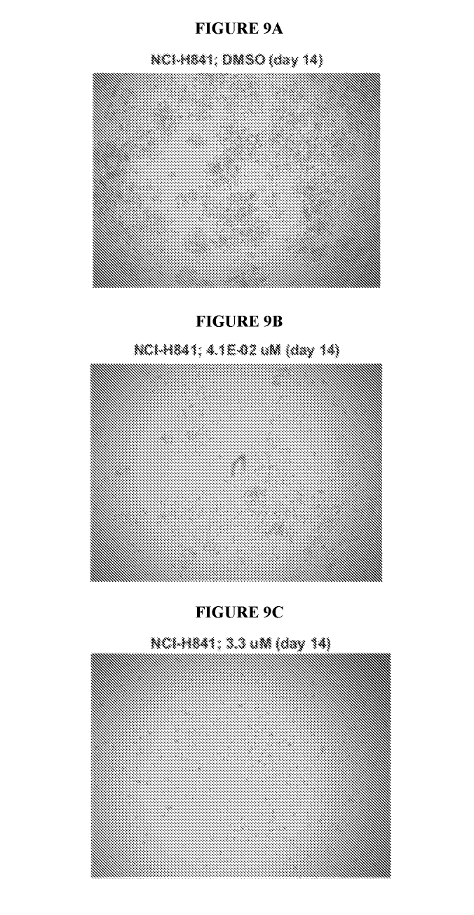

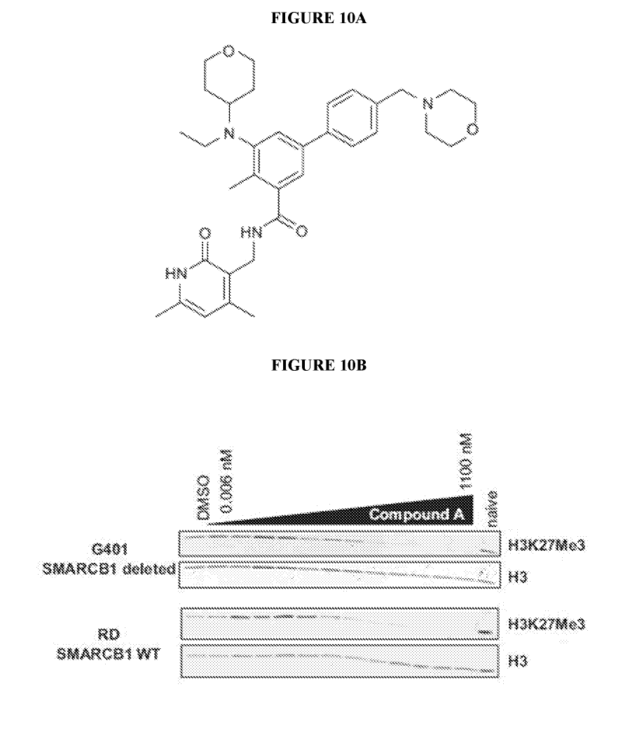

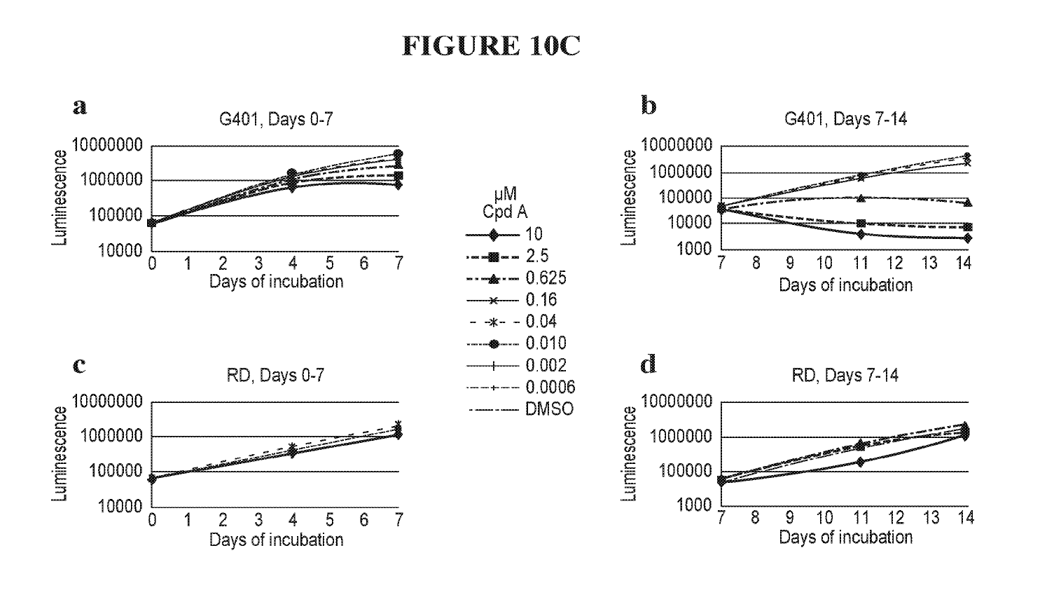

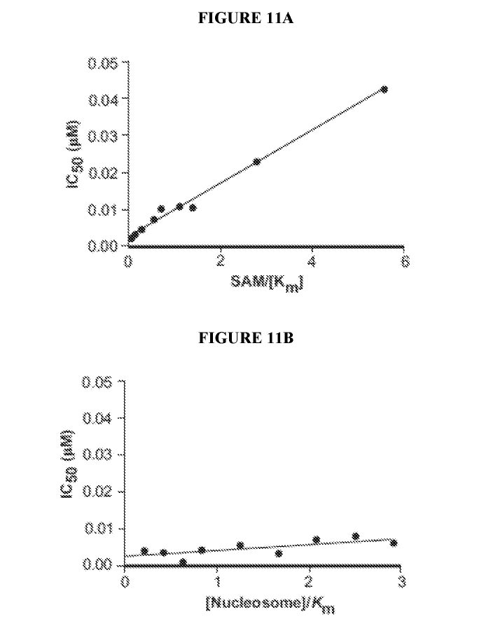

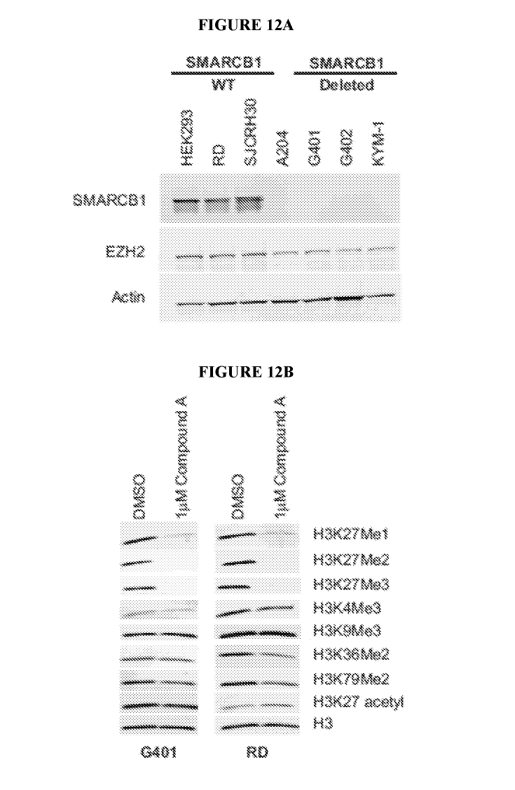

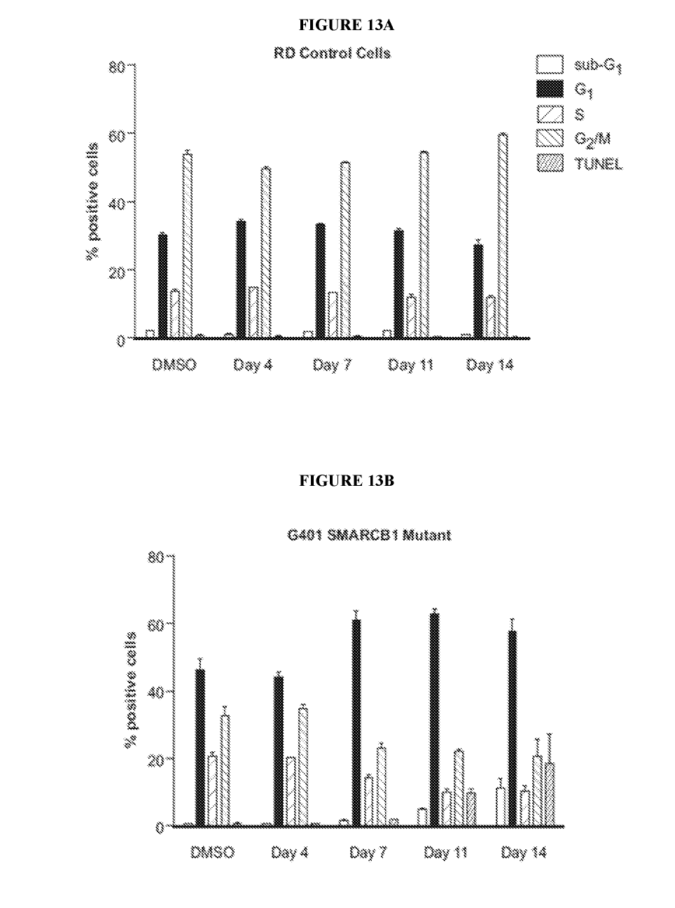

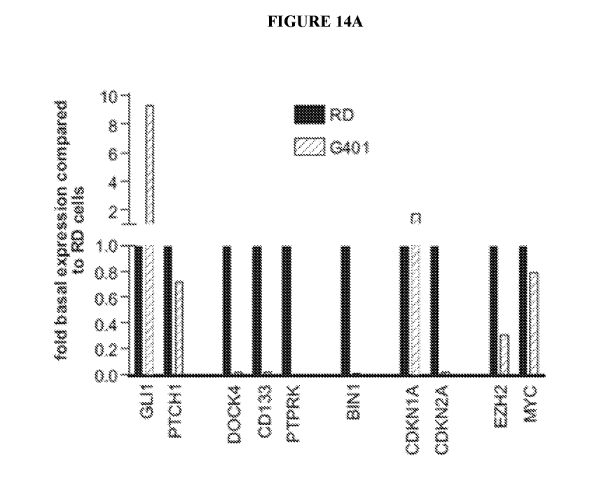

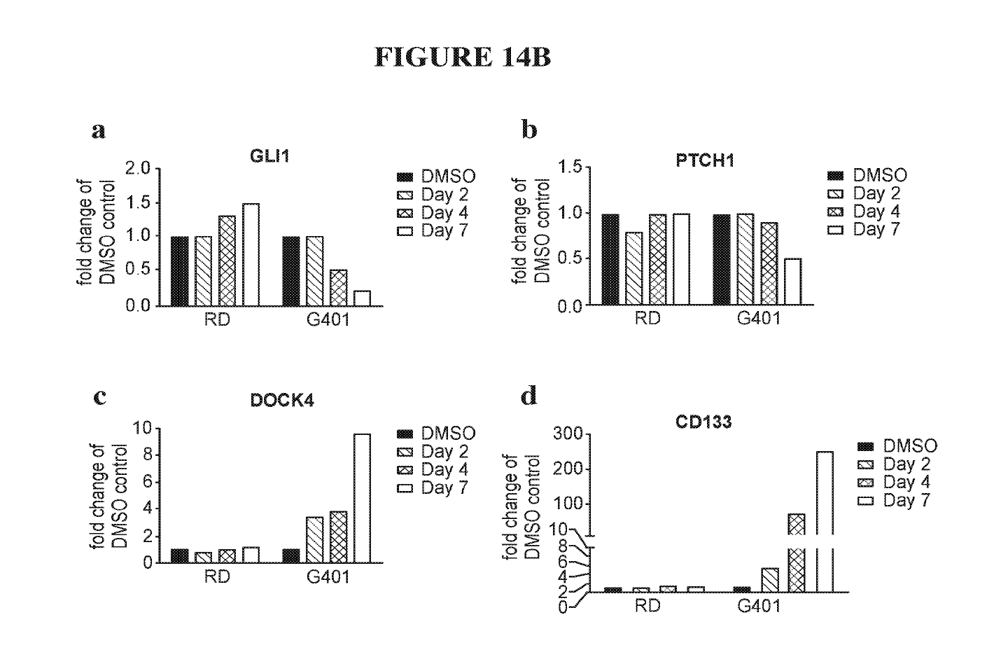

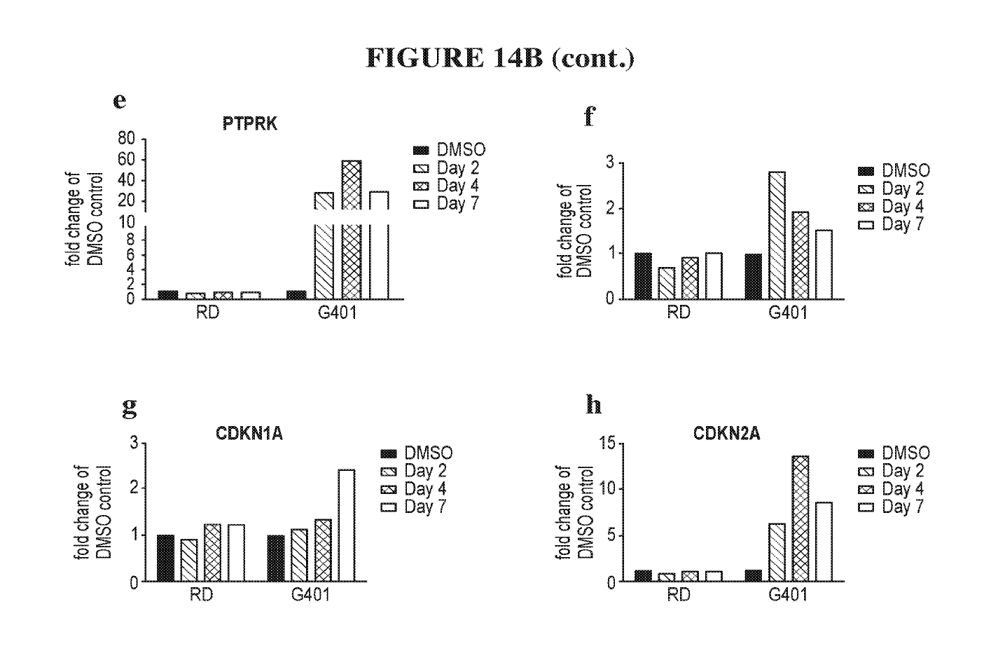

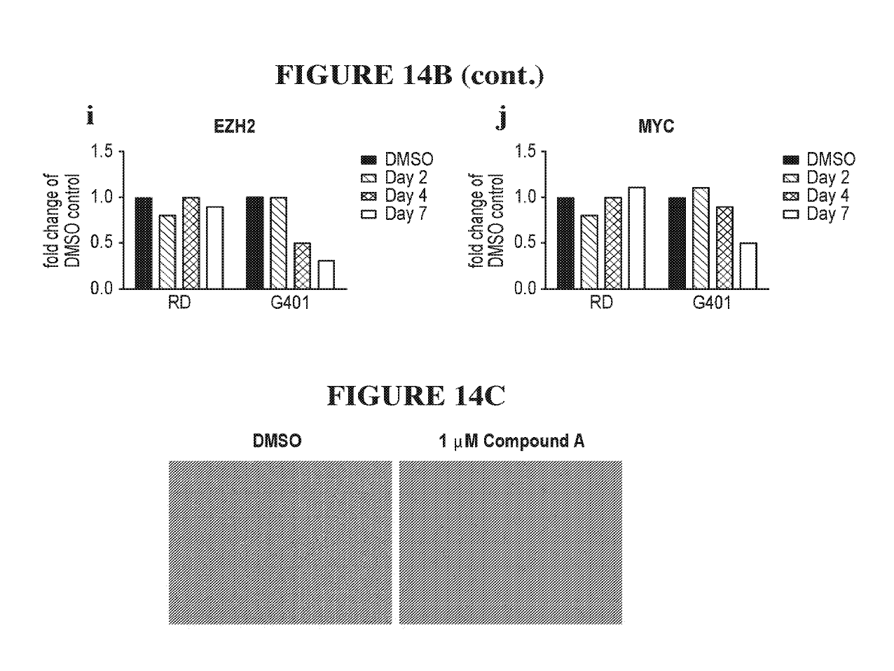

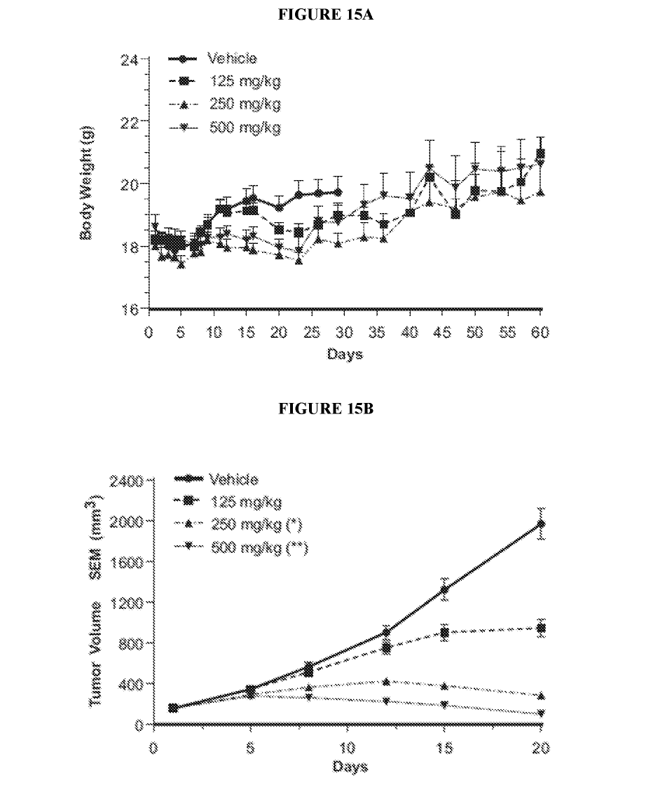

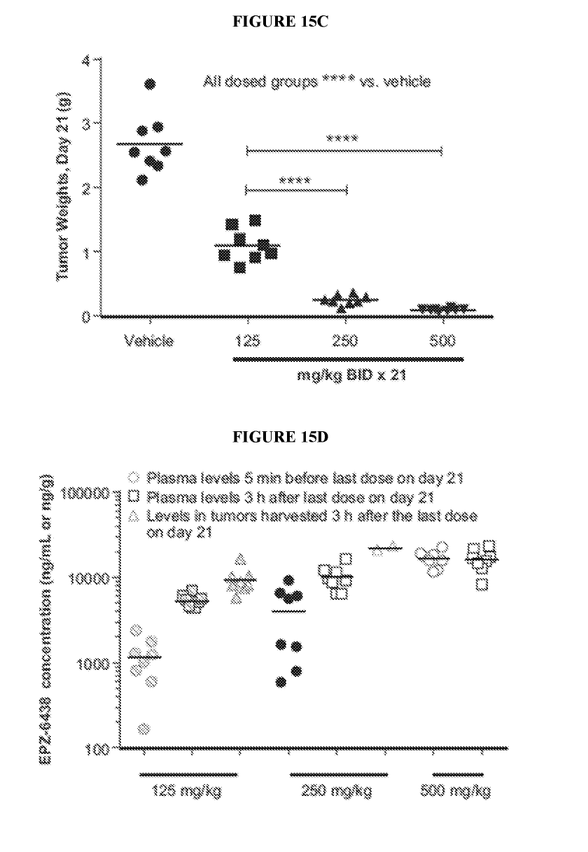

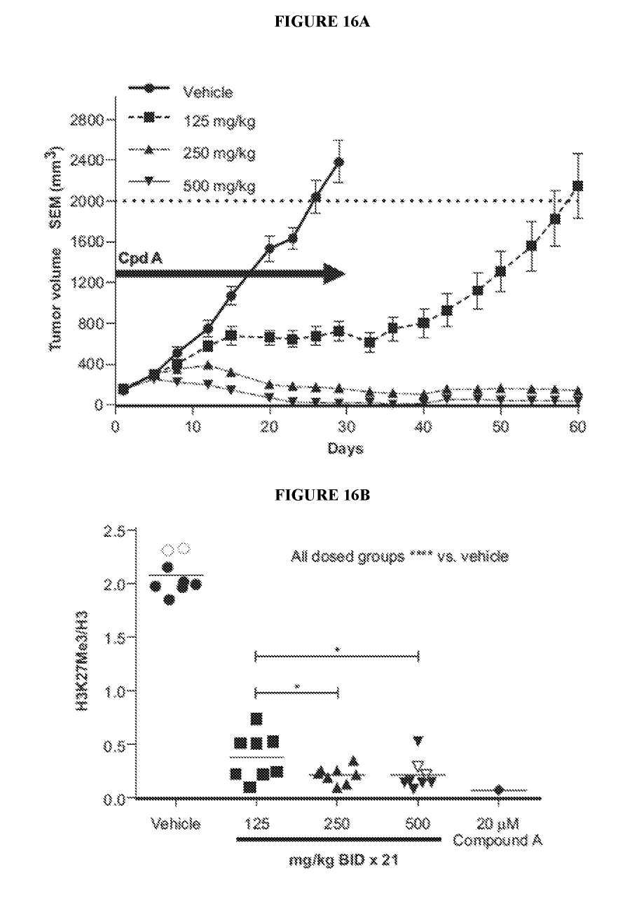

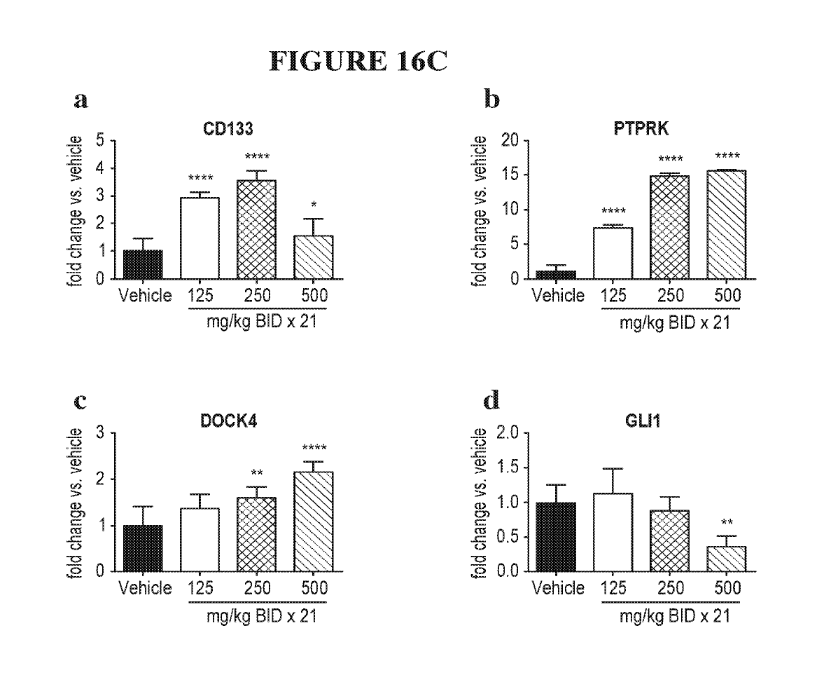

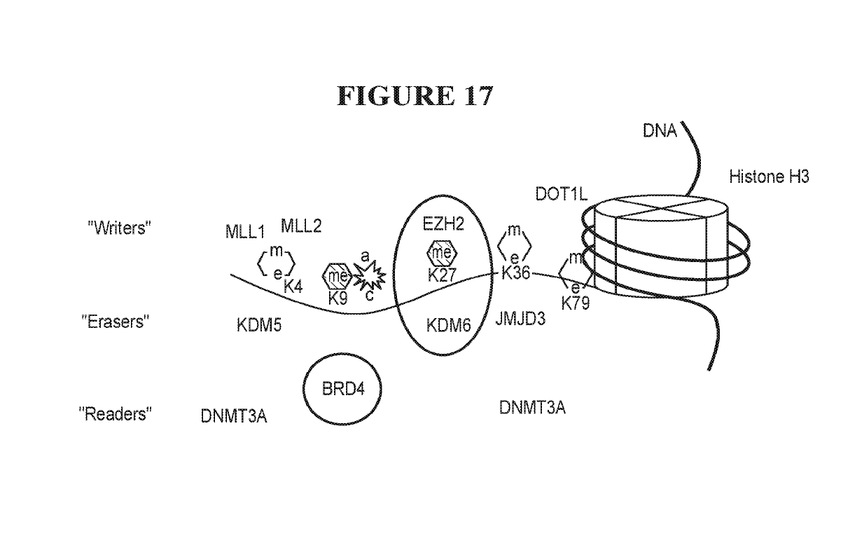

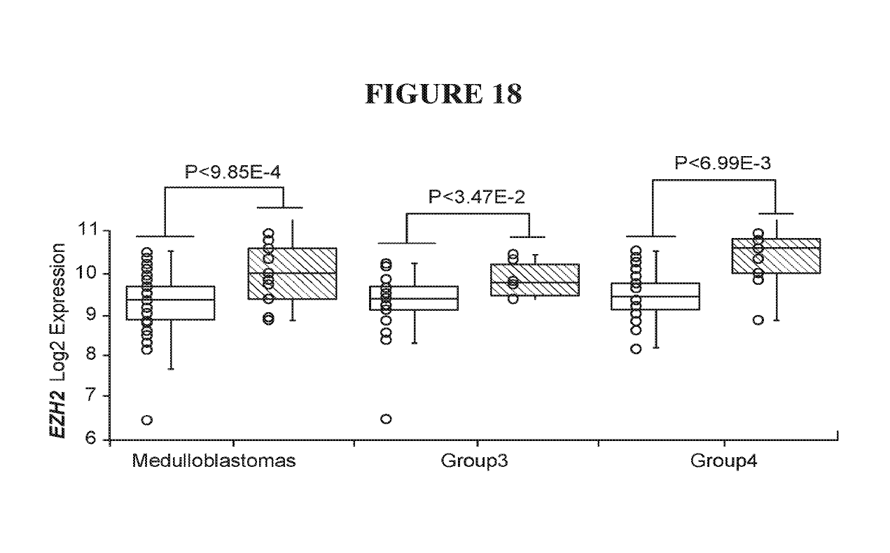

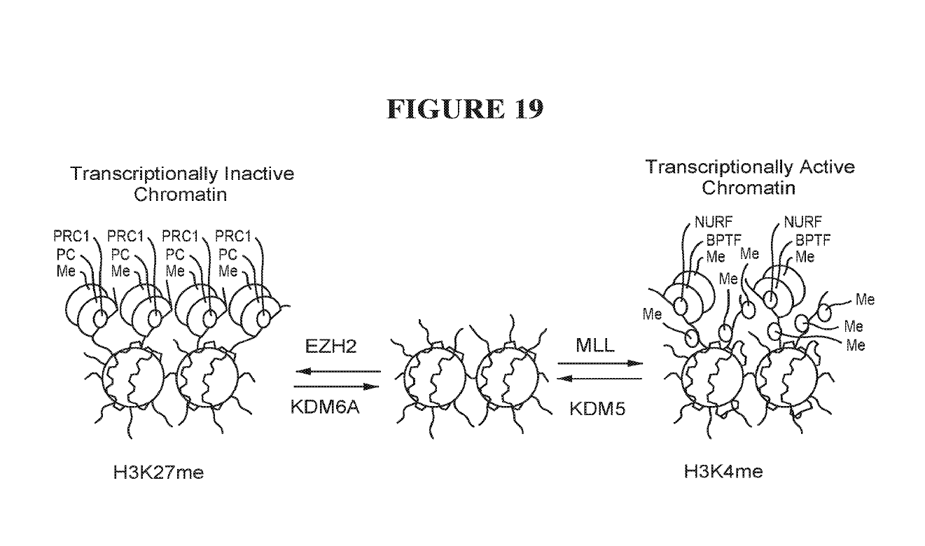

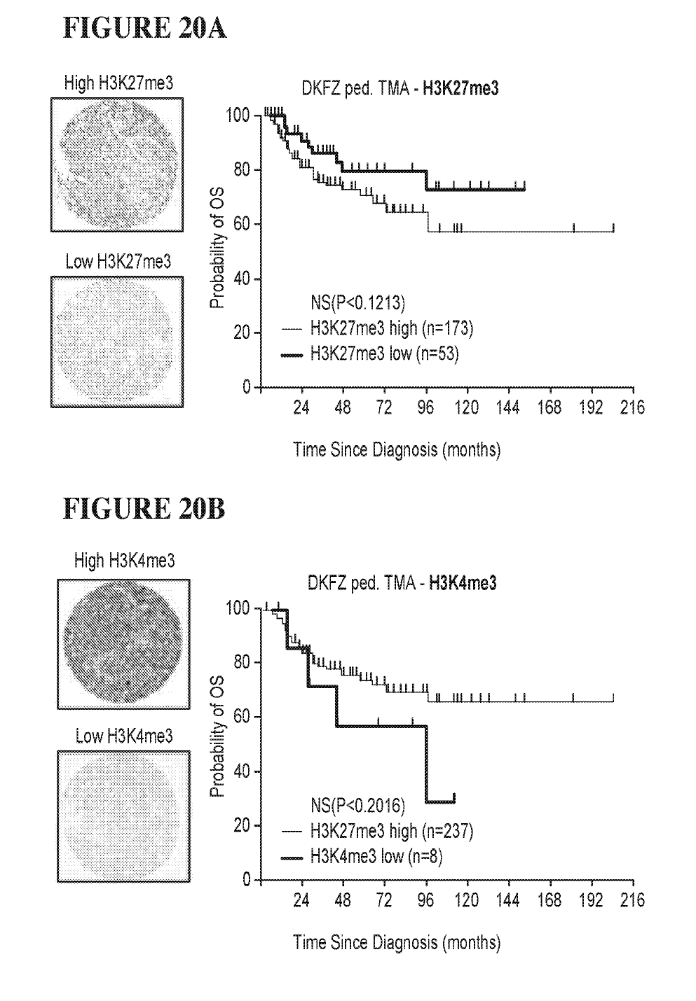

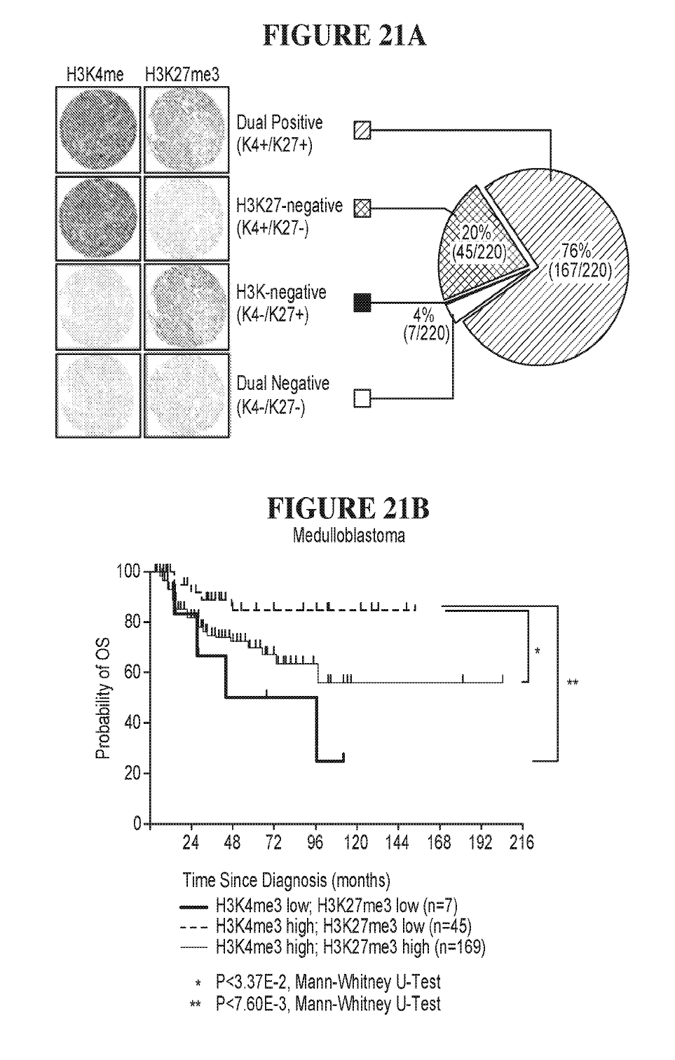

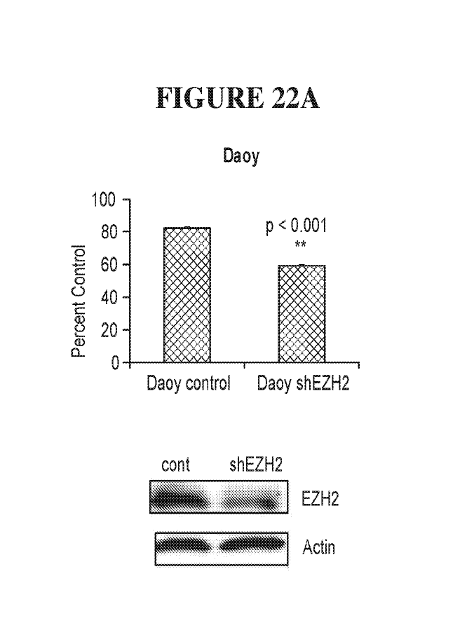

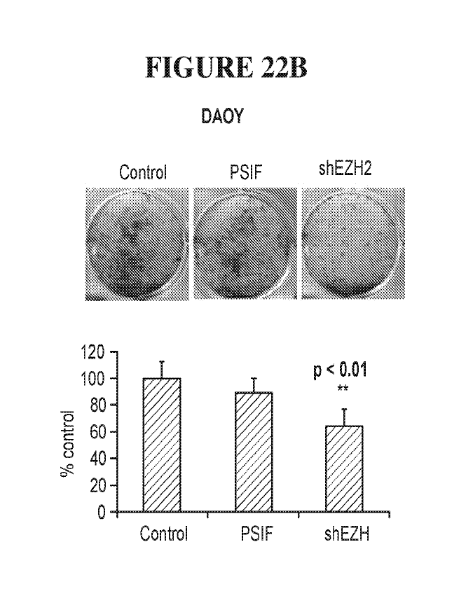

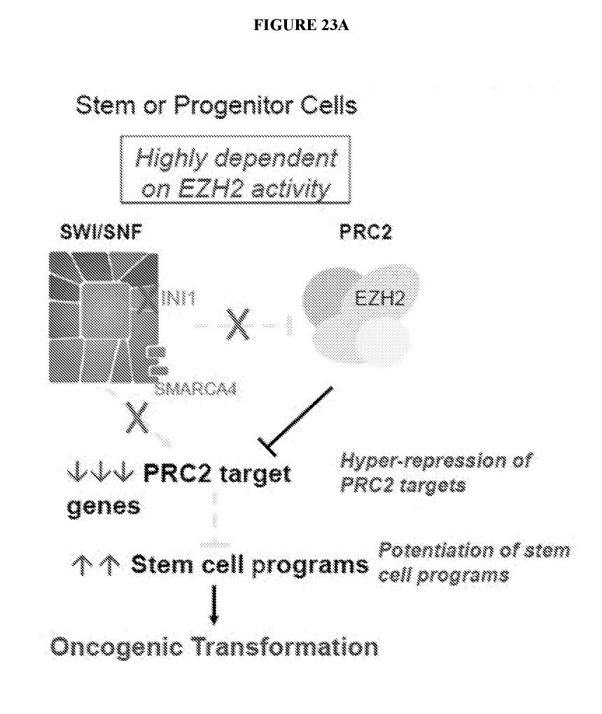

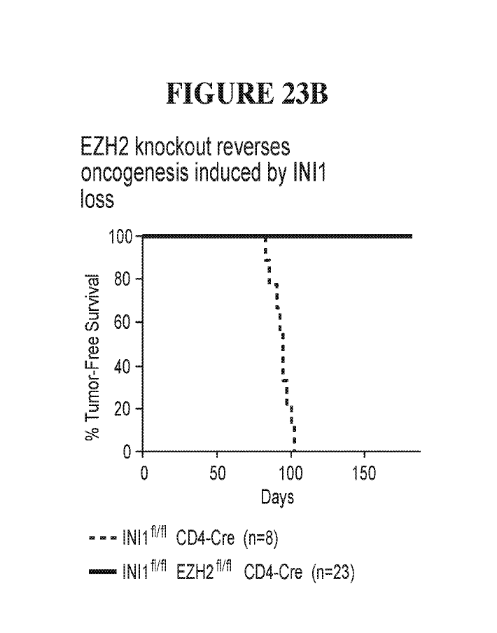

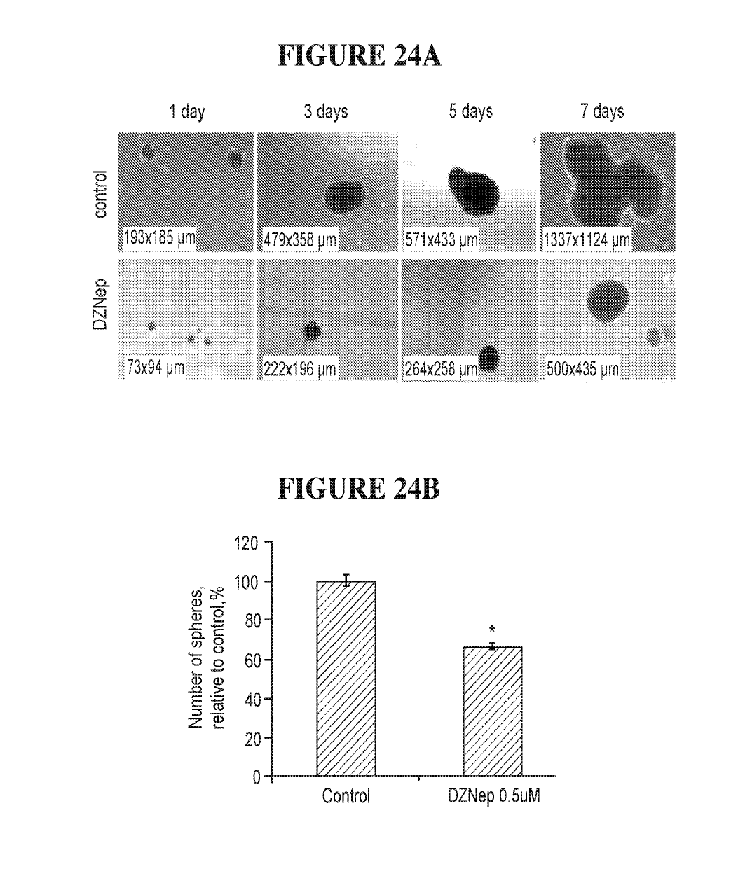

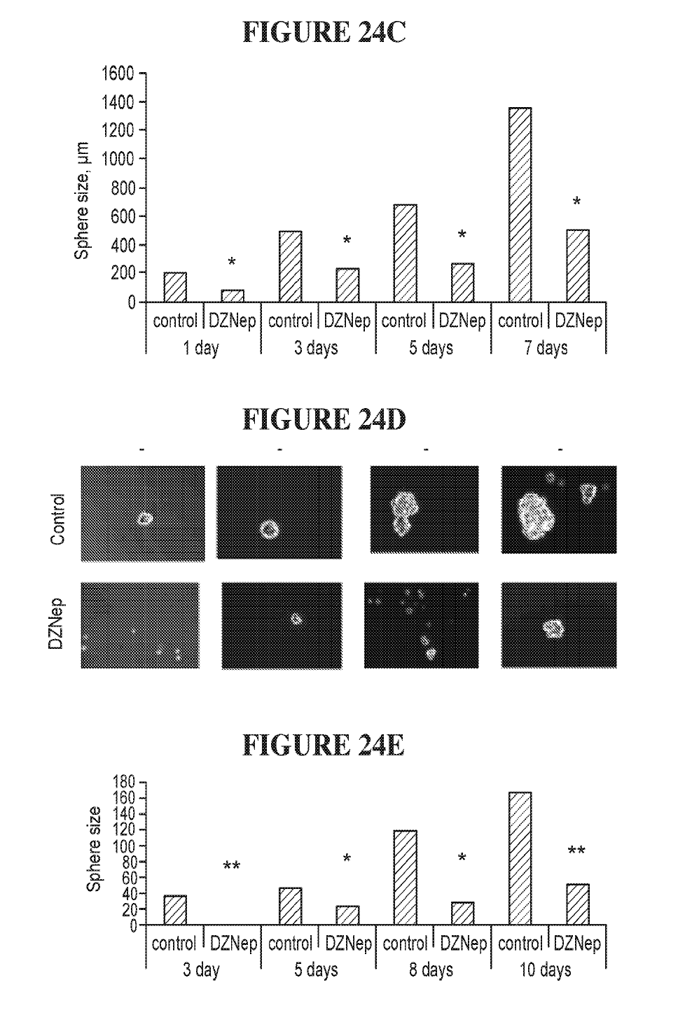

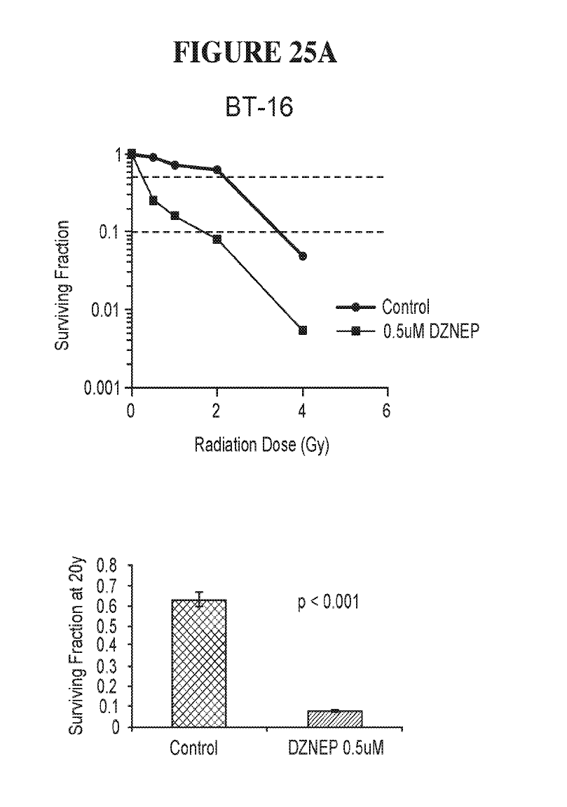

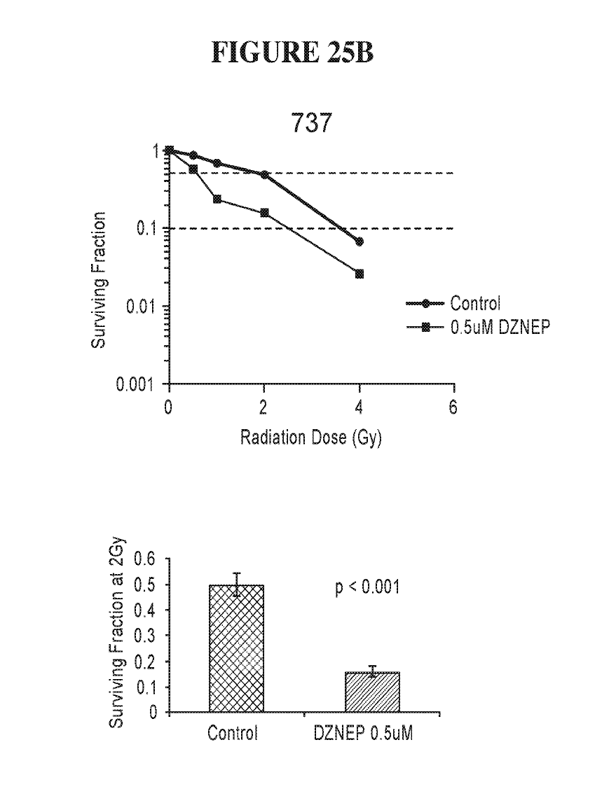

### DETAILED DESCRIPTION

Some aspects of this disclosure provide methods, strategies, and dosing schedules for treating cancer in a subject by administering to the subject a therapeutically-effective amount of an enhancer of a zeste homolog 2 (EZH2) inhibitor. In some embodiments, the cancer is an INI1-deficient tumor. In some embodiments, methods of treating cancer, e.g., an INI1-deficient tumor, of the disclosure may comprise preventing and/or inhibiting proliferation of a malignant cell, e.g., of an INI1-deficient cell.

The disclosure provides a method for treating or alleviating a symptom of a SWI/SNF-associated cancer in a subject by administering to a subject in need thereof a therapeutically effective amount of an EZH2 inhibitor. For example, the SWI/SNF-associated cancer is characterized by reduced expression and/or loss of function of the SWI/SNF complex or one or more components of the SWI/SNF complex. In a preferred embodiment, the cancer is an INI1-deficient tumor

The disclosure also provides a method of treating or alleviating a symptom of a SWI/SNF-associated cancer in a subject in need thereof by (a) determining the expression level of at least one gene selected from the group consisting of differentiation genes, cell cycle inhibition genes and tumor suppressor genes in a sample obtained from the subject; (b) selecting the subject having a decreased expression level of at least one gene in step a; and (c) administering to the subject selected in step b an effective amount of an EZH2 inhibitor, thereby treating or alleviating a symptom of cancer in the subject. In a preferred embodiment, the cancer is an INI1-deficient tumor.

The disclosure further provides a method of treating or alleviating a symptom of a SWI/SNF-associated cancer in a subject in need thereof by (a) determining the expression level of at least one gene selected from the group consisting of hedgehog pathway genes, myc pathway genes and histone methyltransferase genes in a sample obtained from the subject; (b) selecting the subject having an increased expression level of at least one gene in step a; and (c) administering to the subject selected in step b an effective amount of an EZH2 inhibitor, thereby treating or alleviating a symptom of cancer in the subject. In a preferred embodiment, the cancer is an INI1-deficient tumor.

For example, the differentiation gene is CD133, DOCK4, or PTPRK.

For example, the cell cycle inhibition gene is CKDN1A or CDKN2A.

For example, the tumor suppressor gene is BIN1.

For example, the hedgehog pathway gene is GI1 or PTCH1.

For example, the myc pathway gene is MYC.

For example, the histone methyltransferase gene is EZH2.

The disclosure also provides a method of inducing differentiation, cell cycle inhibition or tumor suppression by contacting a cell with an EZH2 inhibitor. The EZH2 inhibitor may be in an amount sufficient to increase expression of at least one gene selected from the group consisting of CD133, DOCK4, PTPRK, CKDN1A, CDKN2A and BIN1.

The disclosure also provides a method of inhibiting hedgehog signaling by contacting a cell with an EZH2 inhibitor. The EZH2 inhibitor can be in an amount sufficient to reduce expression of GLI1 and/or PTCH1.

The disclosure also provides a method of inducing gene expression by contacting a cell with an EZH2 inhibitor. The EZH2 inhibitor can be in an amount sufficient to induce differentiation, cell cycle inhibition and/or tumor suppression. For example, the gene can be CD133, DOCK4, PTPRK, CKDN1A, CKDN2A or BIN1.

The disclosure also provides a method of inhibiting gene expression by contacting a cell with an EZH2 inhibitor. The EZH2 inhibitor is in an amount sufficient to inhibit hedgehog signaling. For example, the gene can be GLI1 or PTCH1.

For example, the cell may have loss of function of SNF5, ARID1A, ATRX, and/or a component of the SWI/SNF complex.

For example, the loss of function is caused by a deletion of SNF5.

For example, the cell is a cancer cell. Preferably, the cancer is an IND-deficient cancer cell.

For example, the EZH2 inhibitor comprises

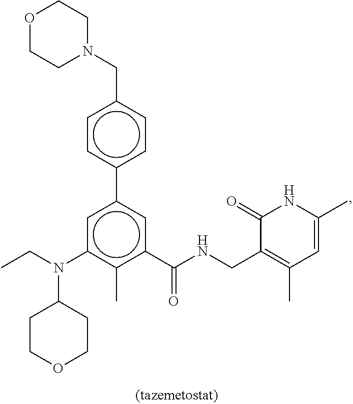

or a pharmaceutically-acceptable salt thereof

For example, the EZH2 inhibitor comprises

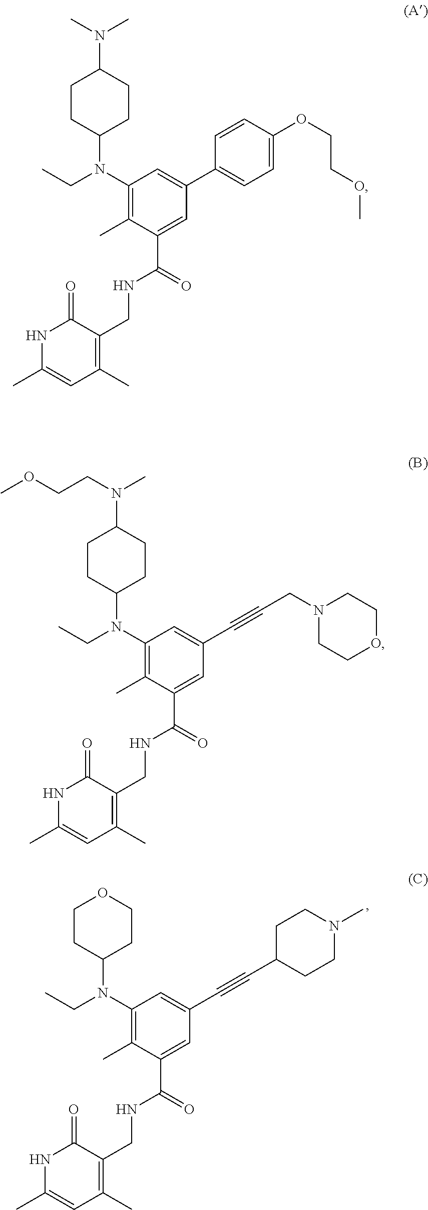

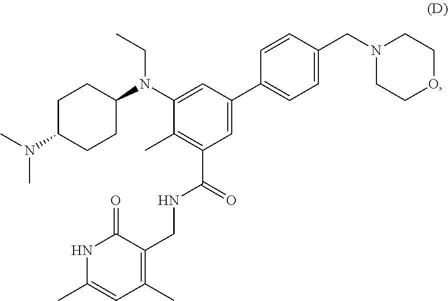 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]

a stereoisomer, a pharmaceutically acceptable salt and/or a solvate thereof

For example, the EZH2 inhibitor comprises

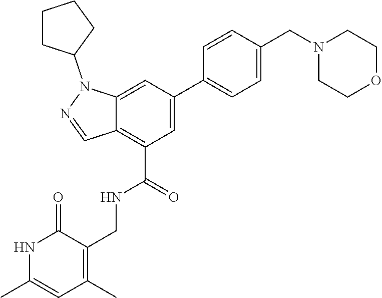 [SMILES: Cc1cc(C)c(CNC(=O)c2cc(-c3ccc(CN4CCOCC4)cc3)cc3c2cnn3C2CCCC2)c(=O)[nH]1]

or a pharmaceutically acceptable salt thereof

For example, the EZH2 inhibitor comprises

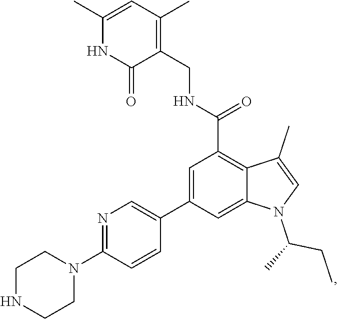 [SMILES: CC[C@H](C)n1cc(C)c2c(C(=O)NCc3c(C)cc(C)[nH]c3=O)cc(-c3ccc(N4CCNCC4)nc3)cc21]

a stereoisomer, a pharmaceutically acceptable salt and/or a solvate thereof

For example, the EZH2 inhibitor comprises

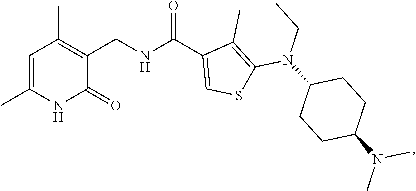

a stereoisomer, a pharmaceutically acceptable salt and/or a solvate thereof.

For example, the EZH2 inhibitor comprises

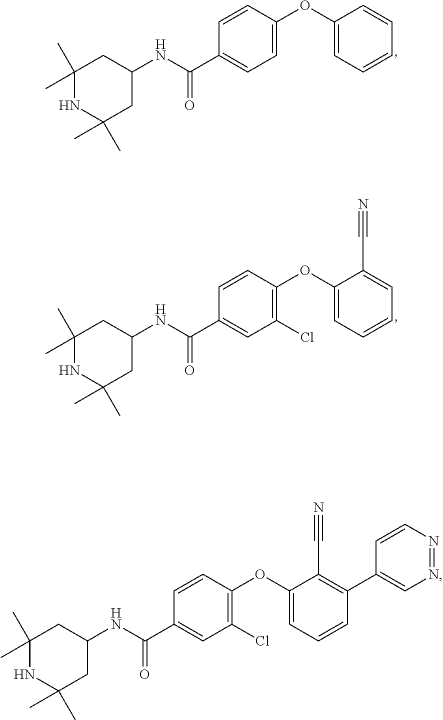 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

a stereoisomer, a pharmaceutically acceptable salt and/or a solvate thereof.

Human nucleic acid and amino acid sequence of components of the SWI/SNF complex have previously been described. See, e.g., GenBank Accession Nos NP_003064.2, NM_003073.3, NP 001007469.1, and NM_001007468.1 for SNFS, GenBank Accession Nos NM_000489.3, NP 000480.2, NM_138270.2, and NP_612114.1 for ATRX, GenBank Accession Nos NP 006006.3, NM_006015.4, NP 624361.1, and NM_139135.2 for ARID1A, each of which is incorporated herein by reference in its entirety.

Spectrum of hSNF5 somatic mutations in human has also been described in Sevenet et al., Human Molecular Genetics, 8: 2359-2368, 1999, which is incorporated herein by reference in its entirety.

A subject in need thereof may have reduced expression, haploinsufficiency, and/or loss of function of SNFS. For example, a subject can comprise a deletion of SNFS in SNFS polypeptide or a nucleic acid sequence encoding a SNFS polypeptide.

```
SWI/SNF-related matrix-associated actin-dependent regulator of chromatin
subfamily B member 1 isoform a (SMARCB1, also called SNF5) [Homo sapiens]
(SEQ ID NO: 1)
      1 mmmmalsktf gqkpvkfqle ddgefymigs evgnylrmfr gslykrypsl wrrlatveer
    61 kkivasshgk ktkpntkdhg yttlatsvtl lkaseveeil dgndekykav sistepptyl
  121 reqkakrnsq wvptlpnssh hldavpcstt inrnrmgrdk krtfplcfdd hdpavihena
  181 sqpevlvpir ldmeidgqkl rdaftwnmne klmtpemfse ilcddldlnp ltfvpaiasa
  241 irqqiesypt dsiledqsdq rviiklnihv gnislvdqfe wdmsekensp ekfalklcse
  301 lglggefvtt iaysirgqls whqktyafse nplptveiai rntgdadqwc plletltdae
  361 mekkirdqdr ntrrmrrlan tapaw
Homo sapiens SWI/SNF related, matrix associated, actin dependent regulator
of chromatin, subfamily b, member 1 (SMARCB1, also called SNF5),
transcript variant 1, mRNA
(SEQ ID NO: 2)
      1 aacgccagcg cctgcgcact gagggcggcc tggtcgtcgt ctgcggcggc ggcggcggct
    61 gaggagcccg gctgaggcgc cagtacccgg cccggtccgc atttcgcctt ccggcttcgg
  121 tttccctcgg cccagcacgc cccggccccg ccccagccct cctgatccct cgcagcccgg
  181 ctccggccgc ccgcctctgc cgccgcaatg atgatgatgg cgctgagcaa gaccttcggg
  241 cagaagcccg tgaagttcca gctggaggac gacggcgagt tctacatgat cggctccgag
  301 gtgggaaact acctccgtat gttccgaggt tctctgtaca agagataccc ctcactctgg
  361 aggcgactag ccactgtgga agagaggaag aaaatagttg catcgtcaca tggtaaaaaa
  421 acaaaaccta acactaagga tcacggatac acgactctag ccaccagtgt gaccctgtta
  481 aaagcctcgg aagtggaaga gattctggat ggcaacgatg agaagtacaa ggctgtgtcc
  541 atcagcacag agccccccac ctacctcagg gaacagaagg ccaagaggaa cagccagtgg
  601 gtacccaccc tgcccaacag ctcccaccac ttagatgccg tgccatgctc cacaaccatc
  661 aacaggaacc gcatgggccg agacaagaag agaaccttcc ccctttgctt tgatgaccat
  721 gacccagctg tgatccatga gaacgcatct cagcccgagg tgctggtccc catccggctg
  781 gacatggaga tcgatgggca gaagctgcga gacgccttca cctggaacat gaatgagaag
  841 ttgatgacgc ctgagatgtt ttcagaaatc ctctgtgacg atctggattt gaacccgctg
  901 acgtttgtgc cagccatcgc ctctgccatc agacagcaga tcgagtccta ccccacggac
  961 agcatcctgg aggaccagtc agaccagcgc gtcatcatca agctgaacat ccatgtggga
1021 aacatttccc tggtggacca gtttgagtgg gacatgtcag agaaggagaa ctcaccagag
1081 aagtttgccc tgaagctgtg ctcggagctg gggttgggcg gggagtttgt caccaccatc
1141 gcatacagca tccggggaca gctgagctgg catcagaaga cctacgcctt cagcgagaac
1201 cctctgccca cagtggagat tgccatccgg aacacgggcg atgcggacca gtggtgccca
1261 ctgctggaga ctctgacaga cgctgagatg gagaagaaga tccgcgacca ggacaggaac
1321 acgaggcgga tgaggcgtct tgccaacacg gccccggcct ggtaaccagc ccatcagcac
1381 acggctccca cggagcatct cagaagattg ggccgcctct cctccatctt ctggcaagga
1441 cagaggcgag gggacagccc agcgccatcc tgaggatcgg gtgggggtgg agtgggggct
1501 tccaggtggc ccttcccggc acacattcca tttgttgagc cccagtcctg ccccccaccc
1561 caccctccct acccctcccc agtctctggg gtcaggaaga aaccttattt taggttgtgt
1621 tttgtttttg tataggagcc ccaggcaggg ctagtaacag tttttaaata aaaggcaaca
1681 ggtcatgttc aatttcttca acaaaaaaaa aaaaaaa
SWI/SNF-related matrix-associated actin-dependent regulator of chromatin
subfamily B member 1 isoform b [Homo sapiens] (SMARCB1, also called SNF5)
(SEQ ID NO: 3)
      1 mmmmalsktf gqkpvkfqle ddgefymigs evgnylrmfr gslykrypsl wrrlatveer
    61 kkivasshdh gyttlatsvt llkaseveei ldgndekyka vsisteppty lreqkakrns
  121 qwvptlpnss hhldavpcst tinrnrmgrd kkrtfplcfd dhdpavihen asqpevlvpi
  181 rldmeidgqk lrdaftwnmn eklmtpemfs eilcddldln pltfvpaias airqqiesyp
  241 tdsiledqsd qrviiklnih vgnisivdqf ewdmsekens pekfalklcs elglggefvt
  301 tiaysirgql swhqktyafs enplptveia irntgdadqw cplletltda emekkirdqd
  361 rntrrmrrla ntapaw
Homo sapiens SWI/SNF related, matrix associated, actin dependent regulator
of chromatin, subfamily b, member 1 (SMARCB1, also called SNF5),transcript
variant2, mRNA
(SEQ ID NO: 4)
      1 aacgccagcg cctgcgcact gagggcggcc tggtcgtcgt ctgcggcggc ggcggcggct
    61 gaggagcccg gctgaggcgc cagtacccgg cccggtccgc atttcgcctt ccggcttcgg
  121 tttccctcgg cccagcacgc cccggccccg ccccagccct cctgatccct cgcagcccgg
  181 ctccggccgc ccgcctctgc cgccgcaatg atgatgatgg cgctgagcaa gaccttcggg
  241 cagaagcccg tgaagttcca gctggaggac gacggcgagt tctacatgat cggctccgag
  301 gtgggaaact acctccgtat gttccgaggt tctctgtaca agagataccc ctcactctgg
  361 aggcgactag ccactgtgga agagaggaag aaaatagttg catcgtcaca tgatcacgga
  421 tacacgactc tagccaccag tgtgaccctg ttaaaagcct cggaagtgga agagattctg
  481 gatggcaacg atgagaagta caaggctgtg tccatcagca cagagccccc cacctacctc
  541 agggaacaga aggccaagag gaacagccag tgggtaccca ccctgcccaa cagctcccac
  601 cacttagatg ccgtgccatg ctccacaacc atcaacagga accgcatggg ccgagacaag
  661 aagagaacct tccccctttg ctttgatgac catgacccag ctgtgatcca tgagaacgca
  721 tctcagcccg aggtgctggt ccccatccgg ctggacatgg agatcgatgg gcagaagctg
  781 cgagacgcct tcacctggaa catgaatgag aagttgatga cgcctgagat gttttcagaa
  841 atcctctgtg acgatctgga tttgaacccg ctgacgtttg tgccagccat cgcctctgcc
  901 atcagacagc agatcgagtc ctaccccacg gacagcatcc tggaggacca gtcagaccag
  961 cgcgtcatca tcaagctgaa catccatgtg ggaaacattt ccctggtgga ccagtttgag
1021 tgggacatgt cagagaagga gaactcacca gagaagtttg ccctgaagct gtgctcggag
1081 ctggggttgg gcggggagtt tgtcaccacc atcgcataca gcatccgggg acagctgagc
1141 tggcatcaga agacctacgc cttcagcgag aaccctctgc ccacagtgga gattgccatc
1201 cggaacacgg gcgatgcgga ccagtggtgc ccactgctgg agactctgac agacgctgag
1261 atggagaaga agatccgcga ccaggacagg aacacgaggc ggatgaggcg tcttgccaac
1321 acggccccgg cctggtaacc agcccatcag cacacggctc ccacggagca tctcagaaga
1381 ttgggccgcc tctcctccat cttctggcaa ggacagaggc gaggggacag cccagcgcca
1441 tcctgaggat cgggtggggg tggagtgggg gcttccaggt ggcccttccc ggcacacatt
1501 ccatttgttg agccccagtc ctgcccccca ccccaccctc cctacccctc cccagtctct
1561 ggggtcagga agaaacctta ttttaggttg tgttttgttt ttgtatagga gccccaggca
1621 gggctagtaa cagtttttaa ataaaaggca acaggtcatg ttcaatttct tcaacaaaaa
1681 aaaaaaaaaa
```

A subject in need thereof may have reduced expression, haploinsufficiency, and/or loss of function of ATRX. For example, a subject can comprise a mutation selected from the group consisting of a substitution of asparagine (N) for the wild type residue lysine (K) at amino acid position 688 of SEQ ID NO: 5 (K688N), and a substitution of isoleucine (I) for the wild type residue methionine (M) at amino acid position 366 of SEQ ID NO: 5 (M366I).

```
Homo sapiens alpha thalassemia/mental retardation syndrome X-linked (ATRX)
isoform 1
(SEQ ID NO: 5)
      1 mtaepmsesk lntlvqklhd flahsseese etsspprlam nqntdkisgs gsnsdmmens
    61 keegtsssek skssgssrsk rkpsivtkyv esddekpldd etvnedasne nsenditmqs
  121 lpkgtvivqp epvlnedkdd fkgpefrsrs kmktenlkkr gedglhgivs ctacgqqvnh
  181 fqkdsiyrhp slqvlicknc fkyymsddis rdsdgmdeqc rwcaeggnli ccdfchnafc
  241 kkciirnlgr kelstimden nqwycyichp eplldlvtac nsvfenleql lqqnkkkikv
  301 dseksnkvye htsrfspkkt ssncngeekk lddscsgsvt ysysalivpk emikkakkli
  361 ettanmnssy vkflkqatdn seissatklr qlkafksvla dikkahlale edlnsefram
  421 davnkekntk ehkvidakfe tkarkgekpc alekkdisks eaklsrkqvd sehmhqnvpt
  481 eeqrtnkstg gehkksdrke epqyepants edldmdivsv pssvpedife nletamevqs
  541 svdhqgdgss gteqevesss vklnisskdn rggiksktta kvtkelyvkl tpvslsnspi
  601 kgadcqevpq dkdgykscgl npklekcglg qensdnehlv enevslllee sdlrrsprvk
  661 ttplrrptet npvtsnsdee cnetvkekqk lsvpvrkkdk rnssdsaidn pkpnklpksk
  721 qsetvdqnsd sdemlailke vsrmshssss dtdineihtn hktlydlktq agkddkgkrk
  781 rksstsgsdf dtkkgksaks siiskkkrqt qsessnydse lekeiksmsk igaarttkkr
  841 ipntkdfdss edekhskkgm dnqghknlkt sqegssddae rkqeretfss aegtvdkdtt
  901 imelrdrlpk kqqasastdg vdklsgkeqs ftslevrkva etkekskhlk tktckkvqdg
  961 lsdiaekflk kdqsdetsed dkkqskkgte ekkkpsdfkk kvikmeqqye sssdgteklp
1021 ereeichfpk gikqikngtt dgekkskkir dktskkkdel sdyaekstgk gdscdssedk
1081 kskngaygre kkrckllgks srkrqdcsss dtekysmked gcnssdkrlk rielrerrnl
1141 sskrntkeiq sgssssdaee ssednkkkkq rtsskkkavi vkekkrnslr tstkrkqadi
1201 tsssssdied ddqnsigegs sdeqkikpvt enlvlsshtg fcqssgdeal sksvpvtvdd
1261 ddddndpenr iakkmlleei kanlssdedg ssddepeegk krtgkqneen pgdeeaknqv
1321 nsesdsdsee skkpryrhrl lrhkltvsdg esgeekktkp kehkevkgrn rrkvssedse
1381 dsdfqesgvs eevsesedeq rprtrsakka eleenqrsyk qkkkrrrikv qedsssenks
1441 nseeeeeeke eeeeeeeeee eeeedendds kspgkgrkki rkilkddklr tetqnalkee
1501 eerrkriaer erereklrev ieiedasptk cpittklvld edeetkeplv qvhrnmvikl
1561 kphqvdgvqf mwdcccesvk ktkkspgsgc ilahcmglgk tlqvvsflht vllcdkldfs
1621 talvvcplnt alnwmnefek wqeglkddek levselatvk rpqersymlq rwqedggvmi
1681 igyemyrnla qgrnvksrkl keifnkalvd pgpdfvvcde ghilkneasa vskamnsirs
1741 rrriiltgtp iqnnlieyhc mvnfikenll gsikefrnrf inpiqngqca dstmvdvrvm
1801 kkrahilyem lagcvqrkdy taltkflppk heyvlavrmt siqcklyqyy ldhltgvgnn
1861 seggrgkaga klfqdfqmls riwthpwclq ldyiskenkg yfdedsmdef iasdsdetsm
1921 slssddytkk kkkgkkgkkd ssssgsgsdn dvevikvwns rsrgggegnv detgnnpsvs
1981 lkleeskats ssnpsspapd wykdfvtdad aevlehsgkm vllfeilrma eeigdkvlvf
2041 sqslisldli edflelasre ktedkdkpli ykgegkwlrn idyyrldgst taqsrkkwae
2101 efndetnvrg rlfiistkag slginlvaan rviifdaswn psydiqsifr vyrfgqtkpv
2161 yvyrflaqgt medkiydrqv tkqslsfrvv dqqqverhft mneltelytf epdllddpns
2221 ekkkkrdtpm lpkdtilael lqihkehivg yhehdslldh keeeelteee rkaawaeyea
2281 ekkgltmrfn iptgtnlppv sfnsqtpyip fnlgalsams nqqledlinq grekvveatn
2341 svtavriqpl ediisavwke nmnlseaqvq alalsrqasq eldvkrreai yndvltkqqm
2401 liscvqrilm nrrlqqqynq qqqqqmtyqq atlghlmmpk ppnlimnpsn yqqidmrgmy
2461 qpvaggmqpp plqrapppmr sknpgpsqgk sm
Homo sapiens alpha thalassemia/mental retardation syndrome X-linked
(ATRX), transcript variant 1, mRNA
(SEQ ID NO: 6)
        1 aattctcctg cctgagcctc ggcccaacaa aatggcggcg gcagcggtgt cgctttgttt
      61 ccgcggctcc tgcggcggtg gcagtggtag cggcctttga gctgtgggga ggttccagca
    121 gcagctacag tgacgactaa gactccagtg catttctatc gtaaccgggc gcgggggagc
    181 gcagatcggc gcccagcaat cacagaagcc gacaaggcgt tcaagcgaaa acatgaccgc
    241 tgagcccatg agtgaaagca agttgaatac attggtgcag aagcttcatg acttccttgc
    301 acactcatca gaagaatctg aagaaacaag ttctcctcca cgacttgcaa tgaatcaaaa
    361 cacagataaa atcagtggtt ctggaagtaa ctctgatatg atggaaaaca gcaaggaaga
    421 gggaactagc tcttcagaaa aatccaagtc ttcaggatcg tcacgatcaa agaggaaacc
    481 ttcaattgta acaaagtatg tagaatcaga tgatgaaaaa cctttggatg atgaaactgt
    541 aaatgaagat gcgtctaatg aaaattcaga aaatgatatt actatgcaga gcttgccaaa
    601 aggtacagtg attgtacagc cagagccagt gctgaatgaa gacaaagatg attttaaagg
    661 gcctgaattt agaagcagaa gtaaaatgaa aactgaaaat ctcaaaaaac gcggagaaga
    721 tgggcttcat gggattgtga gctgcactgc ttgtggacaa caggtcaatc attttcaaaa
    781 agattccatt tatagacacc cttcattgca agttcttatt tgtaagaatt gctttaagta
    841 ttacatgagt gatgatatta gccgtgactc agatggaatg gatgaacaat gtaggtggtg
    901 tgcggaaggt ggaaacttga tttgttgtga cttttgccat aatgctttct gcaagaaatg
    961 cattctacgc aaccttggtc gaaaggagtt gtccacaata atggatgaaa acaaccaatg
  1021 gtattgctac atttgtcacc cagagccttt gttggacttg gtcactgcat gtaacagcgt
  1081 atttgagaat ttagaacagt tgttgcagca aaataagaag aagataaaag ttgacagtga
  1141 aaagagtaat aaagtatatg aacatacatc cagattttct ccaaagaaga ctagttcaaa
  1201 ttgtaatgga gaagaaaaga aattagatga ttcctgttct ggctctgtaa cctactctta
  1261 ttccgcacta attgtgccca aagagatgat taagaaggca aaaaaactga ttgagaccac
  1321 agccaacatg aactccagtt atgttaaatt tttaaagcag gcaacagata attcagaaat
  1381 cagttctgct acaaaattac gtcagcttaa ggcttttaag tctgtgttgg ctgatattaa
  1441 gaaggctcat cttgcattgg aagaagactt aaattccgag tttcgagcga tggatgctgt
  1501 aaacaaagag aaaaatacca aagagcataa agtcatagat gctaagtttg aaacaaaagc
  1561 acgaaaagga gaaaaacctt gtgctttgga aaagaaggat atttcaaagt cagaagctaa
  1621 actttcaaga aaacaggtag atagtgagca catgcatcag aatgttccaa cagaggaaca
  1681 aagaacaaat aaaagtaccg gtggtgaaca taagaaatct gatagaaaag aagaacctca
  1741 atatgaacct gccaacactt ctgaagattt agacatggat attgtgtctg ttccttcctc
  1801 agttccagaa gacatttttg agaatcttga gactgctatg gaagttcaga gttcagttga
  1861 tcatcaaggg gatggcagca gtggaactga acaagaagtg gagagttcat ctgtaaaatt
  1921 aaatatttct tcaaaagaca acagaggagg tattaaatca aaaactacag ctaaagtaac
  1981 aaaagaatta tatgttaaac tcactcctgt ttccctttct aattccccaa ttaaaggtgc
  2041 tgattgtcag gaagttccac aagataaaga tggctataaa agttgtggtc tgaaccccaa
  2101 gttagagaaa tgtggacttg gacaggaaaa cagtgataat gagcatttgg ttgaaaatga
  2161 agtttcatta cttttagagg aatctgatct tcgaagatcc ccacgtgtaa agactacacc
  2221 cttgaggcga ccgacagaaa ctaaccctgt aacatctaat tcagatgaag aatgtaatga
  2281 aacagttaag gagaaacaaa aactatcagt tccagtgaga aaaaaggata agcgtaattc
  2341 ttctgacagt gctatagata atcctaagcc taataaattg ccaaaatcta agcaatcaga
  2401 gactgtggat caaaattcag attctgatga aatgctagca atcctcaaag aggtgagcag
  2461 gatgagtcac agttcttctt cagatactga tattaatgaa attcatacaa accataagac
  2521 tttgtatgat ttaaagactc aggcggggaa agatgataaa ggaaaaagga aacgaaaaag
  2581 ttctacatct ggctcagatt ttgatactaa aaagggcaaa tcagctaaga gctctataat
  2641 ttctaaaaag aaacgacaaa cccagtctga gtcttctaat tatgactcag aattagaaaa
  2701 agagataaag agcatgagta aaattggtgc tgccagaacc accaaaaaaa gaattccaaa
  2761 tacaaaagat tttgactctt ctgaagatga gaaacacagc aaaaaaggaa tggataatca
  2821 agggcacaaa aatttgaaga cctcacaaga aggatcatct gatgatgctg aaagaaaaca
  2881 agagagagag actttctctt cagcagaagg cacagttgat aaagacacga ccatcatgga
  2941 attaagagat cgacttccta agaagcagca agcaagtgct tccactgatg gtgtcgataa
  3001 gctttctggg aaagagcaga gttttacttc tttggaagtt agaaaagttg ctgaaactaa
  3061 agaaaagagc aagcatctca aaaccaaaac atgtaaaaaa gtacaggatg gcttatctga
  3121 tattgcagag aaattcctaa agaaagacca gagcgatgaa acttctgaag atgataaaaa
  3181 gcagagcaaa aagggaactg aagaaaaaaa gaaaccttca gactttaaga aaaaagtaat
  3241 taaaatggaa caacagtatg aatcttcatc tgatggcact gaaaagttac ctgagcgaga
  3301 agaaatttgt cattttccta agggcataaa acaaattaag aatggaacaa ctgatggaga
  3361 aaagaaaagt aaaaaaataa gagataaaac ttctaaaaag aaggatgaat tatctgatta
  3421 tgctgagaag tcaacaggga aaggagatag ttgtgactct tcagaggata aaaagagtaa
  3481 gaatggagca tatggtagag agaagaaaag gtgcaagttg cttggaaaga gttcaaggaa
  3541 gagacaagat tgttcatcat ctgatactga gaaatattcc atgaaagaag atggttgtaa
  3601 ctcttctgat aagagactga aaagaataga attgagggaa agaagaaatt taagttcaaa
  3661 gagaaatact aaggaaatac aaagtggctc atcatcatct gatgctgagg aaagttctga
  3721 agataataaa aagaagaagc aaagaacttc atctaaaaag aaggcagtca ttgtcaagga
  3781 gaaaaagaga aactccctaa gaacaagcac taaaaggaag caagctgaca ttacatcctc
  3841 atcttcttct gatatagaag atgatgatca gaattctata ggtgagggaa gcagcgatga
  3901 acagaaaatt aagcctgtga ctgaaaattt agtgctgtct tcacatactg gattttgcca
  3961 atcttcagga gatgaagcct tatctaaatc agtgcctgtc acagtggatg atgatgatga
  4021 cgacaatgat cctgagaata gaattgccaa gaagatgctt ttagaagaaa ttaaagccaa
  4081 tctttcctct gatgaggatg gatcttcaga tgatgagcca gaagaaggga aaaaaagaac
  4141 tggaaaacaa aatgaagaaa acccaggaga tgaggaagca aaaaatcaag tcaattctga
  4201 atcagattca gattctgaag aatctaagaa gccaagatac agacataggc ttttgcggca
  4261 caaattgact gtgagtgacg gagaatctgg agaagaaaaa aagacaaagc ctaaagagca
  4321 taaagaagtc aaaggcagaa acagaagaaa ggtgagcagt gaagattcag aagattctga
  4381 ttttcaggaa tcaggagtta gtgaagaagt tagtgaatcc gaagatgaac agcggcccag
  4441 aacaaggtct gcaaagaaag cagagttgga agaaaatcag cggagctata aacagaaaaa
  4501 gaaaaggcga cgtattaagg ttcaagaaga ttcatccagt gaaaacaaga gtaattctga
  4561 ggaagaagag gaggaaaaag aagaggagga ggaagaggag gaggaggagg aagaggagga
  4621 ggaagatgaa aatgatgatt ccaagtctcc tggaaaaggc agaaagaaaa ttcggaagat
  4681 tcttaaagat gataaactga gaacagaaac acaaaatgct cttaaggaag aggaagagag
  4741 acgaaaacgt attgctgaga gggagcgtga gcgagaaaaa ttgagagagg tgatagaaat
  4801 tgaagatgct tcacccacca agtgtccaat aacaaccaag ttggttttag atgaagatga
  4861 agaaaccaaa gaacctttag tgcaggttca tagaaatatg gttatcaaat tgaaacccca
  4921 tcaagtagat ggtgttcagt ttatgtggga ttgctgctgt gagtctgtga aaaaaacaaa
  4981 gaaatctcca ggttcaggat gcattcttgc ccactgtatg ggccttggta agactttaca
  5041 ggtggtaagt tttcttcata cagttctttt gtgtgacaaa ctggatttca gcacggcgtt
  5101 agtggtttgt cctcttaata ctgctttgaa ttggatgaat gaatttgaga agtggcaaga
  5161 gggattaaaa gatgatgaga agcttgaggt ttctgaatta gcaactgtga aacgtcctca
  5221 ggagagaagc tacatgctgc agaggtggca agaagatggt ggtgttatga tcataggcta
  5281 tgagatgtat agaaatcttg ctcaaggaag gaatgtgaag agtcggaaac ttaaagaaat
  5341 atttaacaaa gctttggttg atccaggccc tgattttgtt gtttgtgatg aaggccatat
  5401 tctaaaaaat gaagcatctg ctgtttctaa agctatgaat tctatacgat caaggaggag
  5461 gattatttta acaggaacac cacttcaaaa taacctaatt gagtatcatt gtatggttaa
  5521 ttttatcaag gaaaatttac ttggatccat taaggagttc aggaatagat ttataaatcc
  5581 aattcaaaat ggtcagtgtg cagattctac catggtagat gtcagagtga tgaaaaaacg
  5641 tgctcacatt ctctatgaga tgttagctgg atgtgttcag aggaaagatt atacagcatt
  5701 aacaaaattc ttgcctccaa aacacgaata tgtgttagct gtgagaatga cttctattca
  5761 gtgcaagctc tatcagtact acttagatca cttaacaggt gtgggcaata atagtgaagg
  5821 tggaagagga aaggcaggtg caaagctttt ccaagatttt cagatgttaa gtagaatatg
  5881 gactcatcct tggtgtttgc agctagacta cattagcaaa gaaaataagg gttattttga
  5941 tgaagacagt atggatgaat ttatagcctc agattctgat gaaacctcca tgagtttaag
  6001 ctccgatgat tatacaaaaa agaagaaaaa agggaaaaag gggaaaaaag atagtagctc
  6061 aagtggaagt ggcagtgaca atgatgttga agtgattaag gtctggaatt caagatctcg
  6121 gggaggtggt gaaggaaatg tggatgaaac aggaaacaat ccttctgttt ctttaaaact
  6181 ggaagaaagt aaagctactt cttcttctaa tccaagcagc ccagctccag actggtacaa
  6241 agattttgtt acagatgctg atgctgaggt tttagagcat tctgggaaaa tggtacttct
  6301 ctttgaaatt cttcgaatgg cagaggaaat tggggataaa gtccttgttt tcagccagtc
  6361 cctcatatct ctggacttga ttgaagattt tcttgaatta gctagtaggg agaagacaga
  6421 agataaagat aaacccctta tttataaagg tgaggggaag tggcttcgaa acattgacta
  6481 ttaccgttta gatggttcca ctactgcaca gtcaaggaag aagtgggctg aagaatttaa
  6541 tgatgaaact aatgtgagag gacgattatt tatcatttct actaaagcag gatctctagg
  6601 aattaatctg gtagctgcta atcgagtaat tatattcgac gcttcttgga atccatctta
  6661 tgacatccag agtatattca gagtttatcg ctttggacaa actaagcctg tttatgtata
  6721 taggttctta gctcagggaa ccatggaaga taagatttat gatcggcaag taactaagca
  6781 gtcactgtct tttcgagttg ttgatcagca gcaggtggag cgtcatttta ctatgaatga
  6841 gcttactgaa ctttatactt ttgagccaga cttattagat gaccctaatt cagaaaagaa
  6901 gaagaagagg gatactccca tgctgccaaa ggataccata cttgcagagc tccttcagat
  6961 acataaagaa cacattgtag gataccatga acatgattct cttttggacc acaaagaaga
  7021 agaagagttg actgaagaag aaagaaaagc agcttgggct gagtatgaag cagagaagaa
  7081 gggactgacc atgcgtttca acataccaac tgggaccaat ttaccccctg tcagtttcaa
  7141 ctctcaaact ccttatattc ctttcaattt gggagccctg tcagcaatga gtaatcaaca
  7201 gctggaggac ctcattaatc aaggaagaga aaaagttgta gaagcaacaa acagtgtgac
  7261 agcagtgagg attcaacctc ttgaggatat aatttcagct gtatggaagg agaacatgaa
  7321 tctctcagag gcccaagtac aggcgttagc attaagtaga caagccagcc aggagcttga
  7381 tgttaaacga agagaagcaa tctacaatga tgtattgaca aaacaacaga tgttaatcag
  7441 ctgtgttcag cgaatactta tgaacagaag gctccagcag cagtacaatc agcagcaaca
  7501 gcaacaaatg acttatcaac aagcaacact gggtcacctc atgatgccaa agcccccaaa
  7561 tttgatcatg aatccttcta actaccagca gattgatatg agaggaatgt atcagccagt
  7621 ggctggtggt atgcagccac caccattaca gcgtgcacca cccccaatga gaagcaaaaa
  7681 tccaggacct tcccaaggga aatcaatgtg attttgcact aaaagcttaa tggattgtta
  7741 aaatcataga aagatctttt atttttttag gaatcaatga cttaacagaa ctcaactgta
  7801 taaatagttt ggtcccctta aatgccaatc ttccatatta gttttacttt tttttttttt
  7861 aaatagggca taccatttct tcctgacatt tgtcagtgat gttgcctaga atcttcttac
  7921 acacgctgag tacagaagat atttcaaatt gttttcagtg aaaacaagtc cttccataat
  7981 agtaacaact ccacagattt cctctctaaa tttttatgcc tgcttttagc aaccataaaa
  8041 ttgtcataaa attaataaat ttaggaaaga ataaagattt atatattcat tctttacata
  8101 taaaaacaca cagctgagtt cttagagttg attcctcaag ttatgaaata cttttgtact
  8161 taatccattt cttgattaaa gtgattgaaa tggttttaat gttcttttga ctgaagtctg
  8221 aaactgggct cctgctttat tgtctctgtg actgaaagtt agaaactgag ggttatcttt
  8281 gacacagaat tgtgtgcaat attcttaaat actactgctc taaaagttgg agaagtcttg
  8341 cagttatctt agcattgtat aaacagcctt aagtatagcc taagaagaga attccttttt
  8401 cttctttagt ccttctgcca ttttttattt tcagttatat gtgctgaaat aattactggt
  8461 aaaatttcag ggttgtggat tatcttccac acatgaattt tctctctcct ggcacgaata
  8521 taaagcacat ctcttaactg catggtgcca gtgctaatgc ttcatcctgt tgctggcagt
  8581 gggatgtgga cttagaaaat caagttctag cattttagta ggttaacact gaagttgtgg
  8641 ttgttaggtt cacaccctgt tttataaaca acatcaaaat ggcagaacca ttgctgactt
  8701 taggttcaca tgaggaatgt acttttaaca attcccagta ctatcagtat tgtgaaataa
  8761 ttcctctgaa agataagaat cactggcttc tatgcgcttc ttttctctca tcatcatgtt
  8821 cttttacccc agtttcctta cattttttta aattgtttca gagtttgttt tttttttagt
  8881 ttagattgtg aggcaattat taaatcaaaa ttaattcatc caatacccct ttactagaag
  8941 ttttactaga aaatgtatta cattttattt tttcttaatc cagttctgca aaaatgacct
  9001 ataaatttat tcatgtacaa ttttggttac ttgaattgtt aaagaaaaca ttgtttttga
  9061 ctatgggagt caactcaaca tggcagaacc atttttgaga tgatgataca acaggtagtg
  9121 aaacagctta agaattccaa aaaaaaaaaa aaaaaaaaaa aaaagaaaac tgggtttggg
  9181 ctttgcttta ggtatcactg gattagaatg agtttaacat tagctaaaac tgctttgagt
  9241 tgtttggatg attaagagat tgccattttt atcttggaag aactagtggt aaaacatcca
  9301 agagcactag gattgtgata cagaatttgt gaggtttggt ggatccacgc ccctctcccc
  9361 cactttccca tgatgaaata tcactaataa atcctgtata tttagatatt atgctagcca
  9421 tgtaatcaga tttatttaat tgggtggggc aggtgtgtat ttactttaga aaaaatgaaa
  9481 aagacaagat ttatgagaaa tatttgaagg cagtacactc tggccaactg ttaccagttg
  9541 gtatttctac aagttcagaa tattttaaac ctgatttact agacctggga attttcaaca
  9601 tggtctaatt atttactcaa agacatagat gtgaaaattt taggcaacct tctaaatctt
  9661 tttcaccatg gatgaaacta taacttaaag aataatactt agaagggtta attggaaatc
  9721 agagtttgaa ataaaacttg gaccactttg tatacactct tctcacttga cattttagct
  9781 atataatatg tactttgagt ataacatcaa gctttaacaa atatttaaag acaaaaaaat
  9841 cacgtcagta aaatactaaa aggctcattt ttatatttgt tttagatgtt ttaaatagtt
  9901 gcaatggatt aaaaatgatg atttaaaatg ttgcttgtaa tacagttttg cctgctaaat
  9961 tctccacatt ttgtaacctg ttttatttct ttgggtgtaa agcgtttttg cttagtattg
10021 tgatattgta tatgttttgt cccagttgta tagtaatgtt tcagtccatc atccagcttt
10081 ggctgctgaa atcatacagc tgtgaagact tgcctttgtt tctgttagac tgcttttcag
10141 ttctgtattg agtatcttaa gtactgtaga aaagatgtca cttcttcctt taaggctgtt
10201 ttgtaatata tataaggact ggaattgtgt ttttaaagaa aagcattcaa gtatgacaat
10261 atactatctg tgttttcacc attcaaagtg ctgtttagta gttgaaactt aaactattta
10321 atgtcattta ataaagtgac caaaatgtgt tgtgctcttt attgtatttt cacagctttg
10381 aaaatctgtg cacatactgt ttcatagaaa atgtatagct tttgttgtcc tatataatgg
10441 tggttctttt gcacatttag ttatttaata ttgagaggtc acgaagtttg gttattgaat
10501 ctgttatata ctaaattctg taaagggaga tctctcatct caaaaagaat ttacatacca
10561 ggaagtccat gtgtgtttgt gttagttttg gatgtctttg tgtaatccag ccccatttcc
10621 tgtttcccaa cagctgtaac actcatttta agtcaagcag ggctaccaac ccacacttga
10681 tagaaaagct gcttaccatt cagaagcttc cttattacct ggcctccaaa tgagctgaat
10741 attttgtagc cttcccttag ctatgttcat tttccctcca ttatcataaa atcagatcga
10801 tatttatgtg ccccaaacaa aactttaaga gcagttacat tctgtcccag tagcccttgt
10861 ttcctttgag agtagcatgt tgtgaggcta tagagactta ttctaccagt aaaacaggtc
10921 aatcctttta catgtttatt atactaaaaa ttatgttcag ggtatttact actttatttc
10981 accagactca gtctcaagtg acttggctat ctccaaatca gatctaccct tagagaataa
11041 acatttttct accgttattt tttttcaagt ctataatctg agccagtccc aaaggagtga
11101 tcaagtttca gaaatgcttt catcttcaca acattttata tatactatta tatggggtga
11161 ataaagtttt aaatccgaaa tataaaaaaa aaaaaaaaaa aa
Homo sapiens alpha thalassemia/mental retardation syndrome X-linked (ATRX)
isoform 2
(SEQ ID NO: 7)
      1 mtaepmsesk lntlvqklhd flahsseese etsspprlam nqntdkisgs gsnsdmmens
    61 keegtsssek skssgssrsk rkpsivtkyv esddekpldd etvnedasne nsenditmqs
  121 lpkedglhgi vsctacgqqv nhfqkdsiyr hpslqvlick ncfkyymsdd isrdsdgmde
  181 qcrwcaeggn liccdfchna fckkcilrnl grkelstimd ennqwycyic hpeplldlvt
  241 acnsvfenle qllqqnkkki kvdseksnkv yehtsrfspk ktssncngee kklddscsgs
  301 vtysysaliv pkemikkakk liettanmns syvkflkqat dnseissatk lrqlkafksv
  361 ladikkahla leedlnsefr amdavnkekn tkehkvidak fetkarkgek pcalekkdis
  421 kseaklsrkq vdsehmhqnv pteeqrtnks tggehkksdr keepqyepan tsedldmdiv
  481 svpssvpedi fenletamev qssvdhqgdg ssgteqeves ssvklnissk dnrggikskt
  541 takvtkelyv kltpvslsns pikgadcqev pqdkdgyksc glnpklekcg lgqensdneh
  601 lvenevslll eesdlrrspr vkttplrrpt etnpvtsnsd eecnetvkek qklsvpvrkk
  661 dkrnssdsai dnpkpnklpk skqsetvdqn sdsdemlail kevsrmshss ssdtdineih
  721 tnhktlydlk tqagkddkgk rkrksstsgs dfdtkkgksa kssiiskkkr qtqsessnyd
  781 selekeiksm skigaarttk kripntkdfd ssedekhskk gmdnqghknl ktsqegssdd
  841 aerkqeretf ssaegtvdkd ttimelrdrl pkkqqasast dgvdklsgke qsftslevrk
  901 vaetkekskh lktktckkvq dglsdiaekf lkkdqsdets eddkkqskkg teekkkpsdf
  961 kkkvikmeqq yesssdgtek lpereeichf pkgikqikng ttdgekkskk irdktskkkd
1021 elsdyaekst gkgdscdsse dkkskngayg rekkrckllg kssrkrqdcs ssdtekysmk
1081 edgcnssdkr lkrielrerr nlsskrntke iqsgssssda eessednkkk kqrtsskkka
1141 vivkekkrns lrtstkrkqa ditsssssdi edddqnsige gssdeqkikp vtenlvlssh
1201 tgfcqssdge alsksvpvtv ddddddndpe nriakkmlle eikanlssde dgssddepee
1261 gkkrtgkqne enpgdeeakn qvnsesdsds eeskkpryrh rllrhkltvs dgesgeekkt
1321 kpkehkevkg rnrrkvssed sedsdfqesg vseevsesed eqrprtrsak kaeleenqrs
1381 ykqkkkrrri kvqedsssen ksnseeeeee keeeeeeeee eeeeeedend dskspgkgrk
1441 kirkilkddk lrtetqnalk eeeerrkria ererereklr evieiedasp tkcpittklv
1501 ldedeetkep lvqvhrnmvi klkphqvdgv qfmwdccces vkktkkspgs gcilahcmgl
1561 gktlqvvsfl htvllcdkld fstalvvcpl ntalnwmnef ekwqeglkdd eklevselat
1621 vkrpqersym lqrwqedggv miigyemyrn laqgrnvksr klkeifnkal vdpgpdfvvc
1681 deghilknea savskamnsi rsrrriiltg tplqnnliey hcmvnfiken llgsikefrn
1741 rfinpiqngq cadstmvdvr vmkkrahily emlagcvqrk dytaltkflp pkheyvlavr
1801 mtsiqcklyq yyldhltgvg nnseggrgka gaklfqdfqm lsriwthpwc lqldyisken
1861 kgyfdedsmd efiasdsdet smslssddyt kkkkkgkkgk kdssssgsgs dndvevikvw
1921 nsrsrgggeg nvdetgnnps vslkleeska tsssnpsspa pdwykdfvtd adaevlehsg
1981 kmvllfeilr maeeigdkvl vfsqslisld liedflelas rektedkdkp liykgegkwl
2041 rnidyyrldg sttaqsrkkw aeefndetnv rgrlfiistk agslginlva anrviifdas
2101 wnpsydiqsi frvyrfgqtk pvyvyrflaq gtmedkiydr qvtkqslsfr vvdqqqverh
2161 ftmneltely tfepdllddp nsekkkkrdt pmlpkdtila ellqihkehi vgyhehdsll
2221 dhkeeeelte eerkaawaey eaekkgltmr fniptgtnlp pvsfnsqtpy ipfnlgalsa
2281 msnqqiedli nqgrekvvea tnsvtavriq plediisavw kenmnlseaq vqalalsrqa
2341 sqeldvkrre aiyndvltkq qmliscvqri lmnrrlqqqy nqqqqqqmty qqatlghlmm
2401 pkppnlimnp snyqqidmrg myqpvaggmq ppplqrappp mrsknpgpsq gksm
Homo sapiens alpha thalassemia/mental retardation syndrome X-linked (ATRX),
transcript variant 2, mRNA
(SEQ ID NO: 8)
        1 aattctcctg cctgagcctc ggcccaacaa aatggcggcg gcagcggtgt cgctttgttt
      61 ccgcggctcc tgcggcggtg gcagtggtag cggcctttga gctgtgggga ggttccagca
    121 gcagctacag tgacgactaa gactccagtg catttctatc gtaaccgggc gcgggggagc
    181 gcagatcggc gcccagcaat cacagaagcc gacaaggcgt tcaagcgaaa acatgaccgc
    241 tgagcccatg agtgaaagca agttgaatac attggtgcag aagcttcatg acttccttgc
    301 acactcatca gaagaatctg aagaaacaag ttctcctcca cgacttgcaa tgaatcaaaa
    361 cacagataaa atcagtggtt ctggaagtaa ctctgatatg atggaaaaca gcaaggaaga
    421 gggaactagc tcttcagaaa aatccaagtc ttcaggatcg tcacgatcaa agaggaaacc
    481 ttcaattgta acaaagtatg tagaatcaga tgatgaaaaa cctttggatg atgaaactgt
    541 aaatgaagat gcgtctaatg aaaattcaga aaatgatatt actatgcaga gcttgccaaa
    601 agaagatggg cttcatggga ttgtgagctg cactgcttgt ggacaacagg tcaatcattt
    661 tcaaaaagat tccatttata gacacccttc attgcaagtt cttatttgta agaattgctt
    721 taagtattac atgagtgatg atattagccg tgactcagat ggaatggatg aacaatgtag
    781 gtggtgtgcg gaaggtggaa acttgatttg ttgtgacttt tgccataatg ctttctgcaa
    841 gaaatgcatt ctacgcaacc ttggtcgaaa ggagttgtcc acaataatgg atgaaaacaa
    901 ccaatggtat tgctacattt gtcacccaga gcctttgttg gacttggtca ctgcatgtaa
    961 cagcgtattt gagaatttag aacagttgtt gcagcaaaat aagaagaaga taaaagttga
  1021 cagtgaaaag agtaataaag tatatgaaca tacatccaga ttttctccaa agaagactag
  1081 ttcaaattgt aatggagaag aaaagaaatt agatgattcc tgttctggct ctgtaaccta
  1141 ctcttattcc gcactaattg tgcccaaaga gatgattaag aaggcaaaaa aactgattga
  1201 gaccacagcc aacatgaact ccagttatgt taaattttta aagcaggcaa cagataattc
  1261 agaaatcagt tctgctacaa aattacgtca gcttaaggct tttaagtctg tgttggctga
  1321 tattaagaag gctcatcttg cattggaaga agacttaaat tccgagtttc gagcgatgga
  1381 tgctgtaaac aaagagaaaa ataccaaaga gcataaagtc atagatgcta agtttgaaac
  1441 aaaagcacga aaaggagaaa aaccttgtgc tttggaaaag aaggatattt caaagtcaga
  1501 agctaaactt tcaagaaaac aggtagatag tgagcacatg catcagaatg ttccaacaga
  1561 ggaacaaaga acaaataaaa gtaccggtgg tgaacataag aaatctgata gaaaagaaga
  1621 acctcaatat gaacctgcca acacttctga agatttagac atggatattg tgtctgttcc
  1681 ttcctcagtt ccagaagaca tttttgagaa tcttgagact gctatggaag ttcagagttc
  1741 agttgatcat caaggggatg gcagcagtgg aactgaacaa gaagtggaga gttcatctgt
  1801 aaaattaaat atttcttcaa aagacaacag aggaggtatt aaatcaaaaa ctacagctaa
  1861 agtaacaaaa gaattatatg ttaaactcac tcctgtttcc ctttctaatt ccccaattaa
  1921 aggtgctgat tgtcaggaag ttccacaaga taaagatggc tataaaagtt gtggtctgaa
  1981 ccccaagtta gagaaatgtg gacttggaca ggaaaacagt gataatgagc atttggttga
  2041 aaatgaagtt tcattacttt tagaggaatc tgatcttcga agatccccac gtgtaaagac
  2101 tacacccttg aggcgaccga cagaaactaa ccctgtaaca tctaattcag atgaagaatg
  2161 taatgaaaca gttaaggaga aacaaaaact atcagttcca gtgagaaaaa aggataagcg
  2221 taattcttct gacagtgcta tagataatcc taagcctaat aaattgccaa aatctaagca
  2281 atcagagact gtggatcaaa attcagattc tgatgaaatg ctagcaatcc tcaaagaggt
  2341 gagcaggatg agtcacagtt cttcttcaga tactgatatt aatgaaattc atacaaacca
  2401 taagactttg tatgatttaa agactcaggc ggggaaagat gataaaggaa aaaggaaacg
  2461 aaaaagttct acatctggct cagattttga tactaaaaag ggcaaatcag ctaagagctc
  2521 tataatttct aaaaagaaac gacaaaccca gtctgagtct tctaattatg actcagaatt
  2581 agaaaaagag ataaagagca tgagtaaaat tggtgctgcc agaaccacca aaaaaagaat
  2641 tccaaataca aaagattttg actcttctga agatgagaaa cacagcaaaa aaggaatgga
  2701 taatcaaggg cacaaaaatt tgaagacctc acaagaagga tcatctgatg atgctgaaag
  2761 aaaacaagag agagagactt tctcttcagc agaaggcaca gttgataaag acacgaccat
  2821 catggaatta agagatcgac ttcctaagaa gcagcaagca agtgcttcca ctgatggtgt
  2881 cgataagctt tctgggaaag agcagagttt tacttctttg gaagttagaa aagttgctga
  2941 aactaaagaa aagagcaagc atctcaaaac caaaacatgt aaaaaagtac aggatggctt
  3001 atctgatatt gcagagaaat tcctaaagaa agaccagagc gatgaaactt ctgaagatga
  3061 taaaaagcag agcaaaaagg gaactgaaga aaaaaagaaa ccttcagact ttaagaaaaa
  3121 agtaattaaa atggaacaac agtatgaatc ttcatctgat ggcactgaaa agttacctga
  3181 gcgagaagaa atttgtcatt ttcctaaggg cataaaacaa attaagaatg gaacaactga
  3241 tggagaaaag aaaagtaaaa aaataagaga taaaacttct aaaaagaagg atgaattatc
  3301 tgattatgct gagaagtcaa cagggaaagg agatagttgt gactcttcag aggataaaaa
  3361 gagtaagaat ggagcatatg gtagagagaa gaaaaggtgc aagttgcttg gaaagagttc
  3421 aaggaagaga caagattgtt catcatctga tactgagaaa tattccatga aagaagatgg
  3481 ttgtaactct tctgataaga gactgaaaag aatagaattg agggaaagaa gaaatttaag
  3541 ttcaaagaga aatactaagg aaatacaaag tggctcatca tcatctgatg ctgaggaaag
  3601 ttctgaagat aataaaaaga agaagcaaag aacttcatct aaaaagaagg cagtcattgt
  3661 caaggagaaa aagagaaact ccctaagaac aagcactaaa aggaagcaag ctgacattac
  3721 atcctcatct tcttctgata tagaagatga tgatcagaat tctataggtg agggaagcag
  3781 cgatgaacag aaaattaagc ctgtgactga aaatttagtg ctgtcttcac atactggatt
  3841 ttgccaatct tcaggagatg aagccttatc taaatcagtg cctgtcacag tggatgatga
  3901 tgatgacgac aatgatcctg agaatagaat tgccaagaag atgcttttag aagaaattaa
  3961 agccaatctt tcctctgatg aggatggatc ttcagatgat gagccagaag aagggaaaaa
  4021 aagaactgga aaacaaaatg aagaaaaccc aggagatgag gaagcaaaaa atcaagtcaa
  4081 ttctgaatca gattcagatt ctgaagaatc taagaagcca agatacagac ataggctttt
  4141 gcggcacaaa ttgactgtga gtgacggaga atctggagaa gaaaaaaaga caaagcctaa
  4201 agagcataaa gaagtcaaag gcagaaacag aagaaaggtg agcagtgaag attcagaaga
  4261 ttctgatttt caggaatcag gagttagtga agaagttagt gaatccgaag atgaacagcg
  4321 gcccagaaca aggtctgcaa agaaagcaga gttggaagaa aatcagcgga gctataaaca
  4381 gaaaaagaaa aggcgacgta ttaaggttca agaagattca tccagtgaaa acaagagtaa
  4441 ttctgaggaa gaagaggagg aaaaagaaga ggaggaggaa gaggaggagg aggaggaaga
  4501 ggaggaggaa gatgaaaatg atgattccaa gtctcctgga aaaggcagaa agaaaattcg
  4561 gaagattctt aaagatgata aactgagaac agaaacacaa aatgctctta aggaagagga
  4621 agagagacga aaacgtattg ctgagaggga gcgtgagcga gaaaaattga gagaggtgat
  4681 agaaattgaa gatgcttcac ccaccaagtg tccaataaca accaagttgg ttttagatga
  4741 agatgaagaa accaaagaac ctttagtgca ggttcataga aatatggtta tcaaattgaa
  4801 accccatcaa gtagatggtg ttcagtttat gtgggattgc tgctgtgagt ctgtgaaaaa
  4861 aacaaagaaa tctccaggtt caggatgcat tcttgcccac tgtatgggcc ttggtaagac
  4921 tttacaggtg gtaagttttc ttcatacagt tcttttgtgt gacaaactgg atttcagcac
  4981 ggcgttagtg gtttgtcctc ttaatactgc tttgaattgg atgaatgaat ttgagaagtg
  5041 gcaagaggga ttaaaagatg atgagaagct tgaggtttct gaattagcaa ctgtgaaacg
  5101 tcctcaggag agaagctaca tgctgcagag gtggcaagaa gatggtggtg ttatgatcat
  5161 aggctatgag atgtatagaa atcttgctca aggaaggaat gtgaagagtc ggaaacttaa
  5221 agaaatattt aacaaagctt tggttgatcc aggccctgat tttgttgttt gtgatgaagg
  5281 ccatattcta aaaaatgaag catctgctgt ttctaaagct atgaattcta tacgatcaag
  5341 gaggaggatt attttaacag gaacaccact tcaaaataac ctaattgagt atcattgtat
  5401 ggttaatttt atcaaggaaa atttacttgg atccattaag gagttcagga atagatttat
  5461 aaatccaatt caaaatggtc agtgtgcaga ttctaccatg gtagatgtca gagtgatgaa
  5521 aaaacgtgct cacattctct atgagatgtt agctggatgt gttcagagga aagattatac
  5581 agcattaaca aaattcttgc ctccaaaaca cgaatatgtg ttagctgtga gaatgacttc
  5641 tattcagtgc aagctctatc agtactactt agatcactta acaggtgtgg gcaataatag
  5701 tgaaggtgga agaggaaagg caggtgcaaa gcttttccaa gattttcaga tgttaagtag
  5761 aatatggact catccttggt gtttgcagct agactacatt agcaaagaaa ataagggtta
  5821 ttttgatgaa gacagtatgg atgaatttat agcctcagat tctgatgaaa cctccatgag
  5881 tttaagctcc gatgattata caaaaaagaa gaaaaaaggg aaaaagggga aaaaagatag
  5941 tagctcaagt ggaagtggca gtgacaatga tgttgaagtg attaaggtct ggaattcaag
  6001 atctcgggga ggtggtgaag gaaatgtgga tgaaacagga aacaatcctt ctgtttcttt
  6061 aaaactggaa gaaagtaaag ctacttcttc ttctaatcca agcagcccag ctccagactg
  6121 gtacaaagat tttgttacag atgctgatgc tgaggtttta gagcattctg ggaaaatggt
  6181 acttctcttt gaaattcttc gaatggcaga ggaaattggg gataaagtcc ttgttttcag
  6241 ccagtccctc atatctctgg acttgattga agattttctt gaattagcta gtagggagaa
  6301 gacagaagat aaagataaac cccttattta taaaggtgag gggaagtggc ttcgaaacat
  6361 tgactattac cgtttagatg gttccactac tgcacagtca aggaagaagt gggctgaaga
  6421 atttaatgat gaaactaatg tgagaggacg attatttatc atttctacta aagcaggatc
  6481 tctaggaatt aatctggtag ctgctaatcg agtaattata ttcgacgctt cttggaatcc
  6541 atcttatgac atccagagta tattcagagt ttatcgcttt ggacaaacta agcctgttta
  6601 tgtatatagg ttcttagctc agggaaccat ggaagataag atttatgatc ggcaagtaac
  6661 taagcagtca ctgtcttttc gagttgttga tcagcagcag gtggagcgtc attttactat
  6721 gaatgagctt actgaacttt atacttttga gccagactta ttagatgacc ctaattcaga
  6781 aaagaagaag aagagggata ctcccatgct gccaaaggat accatacttg cagagctcct
  6841 tcagatacat aaagaacaca ttgtaggata ccatgaacat gattctcttt tggaccacaa
  6901 agaagaagaa gagttgactg aagaagaaag aaaagcagct tgggctgagt atgaagcaga
  6961 gaagaaggga ctgaccatgc gtttcaacat accaactggg accaatttac cccctgtcag
  7021 tttcaactct caaactcctt atattccttt caatttggga gccctgtcag caatgagtaa
  7081 tcaacagctg gaggacctca ttaatcaagg aagagaaaaa gttgtagaag caacaaacag
  7141 tgtgacagca gtgaggattc aacctcttga ggatataatt tcagctgtat ggaaggagaa
  7201 catgaatctc tcagaggccc aagtacaggc gttagcatta agtagacaag ccagccagga
  7261 gcttgatgtt aaacgaagag aagcaatcta caatgatgta ttgacaaaac aacagatgtt
  7321 aatcagctgt gttcagcgaa tacttatgaa cagaaggctc cagcagcagt acaatcagca
  7381 gcaacagcaa caaatgactt atcaacaagc aacactgggt cacctcatga tgccaaagcc
  7441 cccaaatttg atcatgaatc cttctaacta ccagcagatt gatatgagag gaatgtatca
  7501 gccagtggct ggtggtatgc agccaccacc attacagcgt gcaccacccc caatgagaag
  7561 caaaaatcca ggaccttccc aagggaaatc aatgtgattt tgcactaaaa gcttaatgga
  7621 ttgttaaaat catagaaaga tcttttattt ttttaggaat caatgactta acagaactca
  7681 actgtataaa tagtttggtc cccttaaatg ccaatcttcc atattagttt tacttttttt
  7741 ttttttaaat agggcatacc atttcttcct gacatttgtc agtgatgttg cctagaatct
  7801 tcttacacac gctgagtaca gaagatattt caaattgttt tcagtgaaaa caagtccttc
  7861 cataatagta acaactccac agatttcctc tctaaatttt tatgcctgct tttagcaacc
  7921 ataaaattgt cataaaatta ataaatttag gaaagaataa agatttatat attcattctt
  7981 tacatataaa aacacacagc tgagttctta gagttgattc ctcaagttat gaaatacttt
  8041 tgtacttaat ccatttcttg attaaagtga ttgaaatggt tttaatgttc ttttgactga
  8101 agtctgaaac tgggctcctg ctttattgtc tctgtgactg aaagttagaa actgagggtt
  8161 atctttgaca cagaattgtg tgcaatattc ttaaatacta ctgctctaaa agttggagaa
  8221 gtcttgcagt tatcttagca ttgtataaac agccttaagt atagcctaag aagagaattc
  8281 ctttttcttc tttagtcctt ctgccatttt ttattttcag ttatatgtgc tgaaataatt
  8341 actggtaaaa tttcagggtt gtggattatc ttccacacat gaattttctc tctcctggca
  8401 cgaatataaa gcacatctct taactgcatg gtgccagtgc taatgcttca tcctgttgct
  8461 ggcagtggga tgtggactta gaaaatcaag ttctagcatt ttagtaggtt aacactgaag
  8521 ttgtggttgt taggttcaca ccctgtttta taaacaacat caaaatggca gaaccattgc
  8581 tgactttagg ttcacatgag gaatgtactt ttaacaattc ccagtactat cagtattgtg
  8641 aaataattcc tctgaaagat aagaatcact ggcttctatg cgcttctttt ctctcatcat
  8701 catgttcttt taccccagtt tccttacatt tttttaaatt gtttcagagt ttgttttttt
  8761 tttagtttag attgtgaggc aattattaaa tcaaaattaa ttcatccaat acccctttac
  8821 tagaagtttt actagaaaat gtattacatt ttattttttc ttaatccagt tctgcaaaaa
  8881 tgacctataa atttattcat gtacaatttt ggttacttga attgttaaag aaaacattgt
  8941 ttttgactat gggagtcaac tcaacatggc agaaccattt ttgagatgat gatacaacag
  9001 gtagtgaaac agcttaagaa ttccaaaaaa aaaaaaaaaa aaaaaaaaaa gaaaactggg
  9061 tttgggcttt gctttaggta tcactggatt agaatgagtt taacattagc taaaactgct
  9121 ttgagttgtt tggatgatta agagattgcc atttttatct tggaagaact agtggtaaaa
  9181 catccaagag cactaggatt gtgatacaga atttgtgagg tttggtggat ccacgcccct
  9241 ctcccccact ttcccatgat gaaatatcac taataaatcc tgtatattta gatattatgc
  9301 tagccatgta atcagattta tttaattggg tggggcaggt gtgtatttac tttagaaaaa
  9361 atgaaaaaga caagatttat gagaaatatt tgaaggcagt acactctggc caactgttac
  9421 cagttggtat ttctacaagt tcagaatatt ttaaacctga tttactagac ctgggaattt
  9481 tcaacatggt ctaattattt actcaaagac atagatgtga aaattttagg caaccttcta
  9541 aatctttttc accatggatg aaactataac ttaaagaata atacttagaa gggttaattg
  9601 gaaatcagag tttgaaataa aacttggacc actttgtata cactcttctc acttgacatt
  9661 ttagctatat aatatgtact ttgagtataa catcaagctt taacaaatat ttaaagacaa
  9721 aaaaatcacg tcagtaaaat actaaaaggc tcatttttat atttgtttta gatgttttaa
  9781 atagttgcaa tggattaaaa atgatgattt aaaatgttgc ttgtaataca gttttgcctg
  9841 ctaaattctc cacattttgt aacctgtttt atttctttgg gtgtaaagcg tttttgctta
  9901 gtattgtgat attgtatatg ttttgtccca gttgtatagt aatgtttcag tccatcatcc
  9961 agctttggct gctgaaatca tacagctgtg aagacttgcc tttgtttctg ttagactgct
10021 tttcagttct gtattgagta tcttaagtac tgtagaaaag atgtcacttc ttcctttaag
10081 gctgttttgt aatatatata aggactggaa ttgtgttttt aaagaaaagc attcaagtat
10141 gacaatatac tatctgtgtt ttcaccattc aaagtgctgt ttagtagttg aaacttaaac
10201 tatttaatgt catttaataa agtgaccaaa atgtgttgtg ctctttattg tattttcaca
10261 gctttgaaaa tctgtgcaca tactgtttca tagaaaatgt atagcttttg ttgtcctata
10321 taatggtggt tcttttgcac atttagttat ttaatattga gaggtcacga agtttggtta
10381 ttgaatctgt tatatactaa attctgtaaa gggagatctc tcatctcaaa aagaatttac
10441 ataccaggaa gtccatgtgt gtttgtgtta gttttggatg tctttgtgta atccagcccc
10501 atttcctgtt tcccaacagc tgtaacactc attttaagtc aagcagggct accaacccac
10561 acttgataga aaagctgctt accattcaga agcttcctta ttacctggcc tccaaatgag
10621 ctgaatattt tgtagccttc ccttagctat gttcattttc cctccattat cataaaatca
10681 gatcgatatt tatgtgcccc aaacaaaact ttaagagcag ttacattctg tcccagtagc
10741 ccttgtttcc tttgagagta gcatgttgtg aggctataga gacttattct accagtaaaa
10801 caggtcaatc cttttacatg tttattatac taaaaattat gttcagggta tttactactt
10861 tatttcacca gactcagtct caagtgactt ggctatctcc aaatcagatc tacccttaga
10921 gaataaacat ttttctaccg ttattttttt tcaagtctat aatctgagcc agtcccaaag
10981 gagtgatcaa gtttcagaaa tgctttcatc ttcacaacat tttatatata ctattatatg
11041 gggtgaataa agttttaaat ccgaaatata aaaaaaaaaa aaaaaaaa
```

A subject in need thereof may have reduced expression, haploinsufficiency, and/or loss of function of ARID1A. For example, a subject may comprise a mutation selected from the group consisting of a nonsense mutation for the wild type residue cysteine (C) at amino acid position 884 of SEQ ID NO: 11 (C884*), a substitution of lysine (K) for the wild type residue glutamic acid (E) at amino acid position 966 (E966K), a nonsense mutation for the wild type residue glutamine (Q) at amino acid position 1411 of SEQ ID NO: 11 (Q1411*), a frame shift mutation at the wild type residue phenylalanine (F) at amino acid position 1720 of SEQ ID NO: 11 (F1720fs), a frame shift mutation after the wild type residue glycine (G) at amino acid position 1847 of SEQ ID NO: 11 (G1847fs), a frame shift mutation at the wild type residue cysteine (C) at amino acid position 1874 of SEQ ID NO: 11 (C1874fs), a substitution of glutamic acid (E) for the wild type residue aspartic acid (D) at amino acid position 1957 (D1957E), a nonsense mutation for the wild type residue glutamine (Q) at amino acid position 1430 of SEQ ID NO: 11 (Q1430*), a frame shift mutation at the wild type residue arginine (R) at amino acid position 1721 of SEQ ID NO: 11 (R1721fs), a substitution of glutamic acid (E) for the wild type residue glycine (G) at amino acid position 1255 (G1255E), a frame shift mutation at the wild type residue glycine (G) at amino acid position 284 of SEQ ID NO: 11 (G284fs), a nonsense mutation for the wild type residue arginine (R) at amino acid position 1722 of SEQ ID NO: 11 (R1722*), a frame shift mutation at the wild type residue methionine (M) at amino acid position 274 of SEQ ID NO: 11 (M274fs), a frame shift mutation at the wild type residue glycine (G) at amino acid position 1847 of SEQ ID NO: 11 (G1847fs), a frame shift mutation at the wild type residue P at amino acid position 559 of SEQ ID NO: 11 (P559fs), a nonsense mutation for the wild type residue arginine (R) at amino acid position 1276 of SEQ ID NO: 11 (R1276*), a frame shift mutation at the wild type residue glutamine (Q) at amino acid position 2176 of SEQ ID NO: 11 (Q2176fs), a frame shift mutation at the wild type residue histidine (H) at amino acid position 203 of SEQ ID NO: 11 (H203fs), a frame shift mutation at the wild type residue alanine (A) at amino acid position 591 of SEQ ID NO: 11 (A591fs), a nonsense mutation for the wild type residue glutamine (Q) at amino acid position 1322 of SEQ ID NO: 11 (Q1322*), a nonsense mutation for the wild type residue serine (S) at amino acid position 2264 of SEQ ID NO: 11 (S2264*), a nonsense mutation for the wild type residue glutamine (Q) at amino acid position 586 of SEQ ID NO: 11 (Q586*), a frame shift mutation at the wild type residue glutamine (Q) at amino acid position 548 of SEQ ID NO: 11 (Q548fs), and a frame shift mutation at the wild type residue asparagine (N) at amino acid position 756 of SEQ ID NO: 11 (N756fs). “*” used herein refers to a stop codon. “fs” used herein refers to a frame shift.

```
AT-rich interactive domain-containing protein 1A (ARID1A) isoform a
[Homo sapiens]
(SEQ ID NO: 9)
      1 maaqvapaaa sslgnppppp pselkkaeqq qreeaggeaa aaaaaergem kaaagqeseg
    61 pavgppqplg kelqdgaesn gggggggags gggpgaepdl knsngnagpr palnnnltep
  121 pggggggssd gvgapphsaa aalpppaygf gqpygrspsa vaaaaaavfh qqhggqqspg
  181 laalqsgggg glepyagpqq nshdhgfpnh qynsyypnrs aypppapaya lssprggtpg
  241 sgaaaaagsk pppsssasas sssssfaqqr fgamggggps aagggtpqpt atptlnwllt
  301 spssargyqg ypggdysggp qdggagkgpa dmasqcwgaa aaaaaaaaas ggaqqrshha
  361 pmspgssggg gqplartpqp sspmdqmgkm rpqpyggtnp ysqqqgppsg pqqghgypgq
  421 pygsqtpqry pmtmqgraqs amgglsytqq ippygqqgps gygqqgqtpy ynqqsphpqq
  481 qqppysqqpp sqtphaqpsy qqqpqsqppq lqssqppysq qpsqpphqqs papypsqqst
  541 tqqhpqsqpp ysqpqaqspy qqqqpqqpap stlsqqaayp qpqsqqsqqt aysqqrfppp
  601 qelsqdsfgs qassapsmts skggqedmnl slqsrpsslp dlsgsiddlp mgtegalspg
  661 vstsgisssq geqsnpaqsp fsphtsphlp girgpspspv gspasvaqsr sgplspaavp
  721 gnqmpprpps gqsdsimhps mnqssiaqdr gymqrnpqmp qysspqpgsa lsprqpsggq
  781 ihtgmgsyqq nsmgsygpqg gqygpqggyp rqpnynalpn anypsagmag ginpmgaggq
  841 mhgqpgippy gtlppgrmsh asmgnrpygp nmanmppqvg sgmcpppggm nrktqetava
  901 mhvaansiqn rppgypnmnq ggmmgtgppy gqginsmagm inpqgppysm ggtmannsag
  961 maaspemmgl gdvkltpatk mnnkadgtpk teskskksss stttnekitk lyelggeper
1021 kmwvdrylaf teekamgmtn lpavgrkpld lyrlyvsvke iggltqvnkn kkwrelatnl
1081 nvgtsssaas slkkqyiqcl yafeckierg edpppdifaa adskksqpki qppspagsgs
1141 mqgpqtpqst sssmaeggdl kpptpastph sqipplpgms rsnsvgiqda fndgsdstfq
1201 krnsmtpnpg yqpsmntsdm mgrmsyepnk dpygsmrkap gsdpfmssgq gpnggmgdpy
1261 sraagpglgn vamgprqhyp yggpydrvrt epgigpegnm stgapqpnlm psnpdsgmys
1321 psryppqqqq qqqqrhdsyg nqfstqgtps gspfpsqqtt myqqqqqnyk rpmdgtygpp
1381 akrhegemys vpystgqgqp qqqqlppaqp qpasqqqaaq pspqqdvynq ygnaypatat
1441 aaterrpagg pqnqfpfqfg rdrvsappgt naqqnmppqm mggpiqasae vaqqgtmwqg
1501 rndmtynyan rqstgsapqg payhgvnrtd emlhtdqran hegswpshgt rqppygpsap
1561 vppmtrppps nyqpppsmqn hipqvsspap lprpmenrts pskspflhsg mkmqkagppv
1621 pashiapapv qppmirrdit fppgsveatq pvlkqrrrlt mkdigtpeaw rvmmslksgl
1681 laestwaldt inillyddns imtfnlsqlp gllellveyf rrclieifgi lkeyevgdpg
1741 qrtlldpgrf skvsspapme ggeeeeellg pkleeeeeee vvendeeiaf sgkdkpasen
1801 seekliskfd klpvkivqkn dpfvvdcsdk lgrvqefdsg llhwrigggd ttehiqthfe
1861 sktellpsrp hapcppaprk hvttaegtpg ttdqegpppd gppekritat mddmlstrss
1921 tltedgakss eaikesskfp fgispaqshr nikiledeph skdetplctl ldwqdslakr
1981 cvcvsntirs lsfvpgndfe mskhpgllli lgklillhhk hperkqaplt yekeeeqdqg
2041 vscnkvewww dclemlrent lvtlanisgq ldlspypesi clpvldgllh wavcpsaeaq
2101 dpfstlgpna vlspqrlvle tlsklsiqdn nvdlilatpp fsrleklyst mvrflsdrkn
2161 pvcremavvl lanlaqgdsl aaraiavqkg signllgfle dslaatqfqq sqasllhmqn
2221 ppfeptsvdm mrraaralla lakvdenhse ftlyesrlld isvsplmnsl vsqvicdvlf
2281 ligqs
Homo sapiens AT rich interactive domain 1A (SWI-like) (ARID1A),
transcript variant 1, mRNA
(SEQ ID NO: 10)
      1 cagaaagcgg agagtcacag cggggccagg ccctggggag cggagcctcc accgcccccc
    61 tcattcccag gcaagggctt ggggggaatg agccgggaga gccgggtccc gagcctacag
  121 agccgggagc agctgagccg ccggcgcctc ggccgccgcc gccgcctcct cctcctccgc
  181 cgccgccagc ccggagcctg agccggcggg gcggggggga gaggagcgag cgcagcgcag
  241 cagcggagcc ccgcgaggcc cgcccgggcg ggtggggagg gcagcccggg ggactgggcc
  301 ccggggcggg gtgggagggg gggagaagac gaagacaggg ccgggtctct ccgcggacga
  361 gacagcgggg atcatggccg cgcaggtcgc ccccgccgcc gccagcagcc tgggcaaccc
  421 gccgccgccg ccgccctcgg agctgaagaa agccgagcag cagcagcggg aggaggcggg
  481 gggcgaggcg gcggcggcgg cagcggccga gcgcggggaa atgaaggcag ccgccgggca
  541 ggaaagcgag ggccccgccg tggggccgcc gcagccgctg ggaaaggagc tgcaggacgg
  601 ggccgagagc aatgggggtg gcggcggcgg cggagccggc agcggcggcg ggcccggcgc
  661 ggagccggac ctgaagaact cgaacgggaa cgcgggccct aggcccgccc tgaacaataa
  721 cctcacggag ccgcccggcg gcggcggtgg cggcagcagc gatggggtgg gggcgcctcc
  781 tcactcagcc gcggccgcct tgccgccccc agcctacggc ttcgggcaac cctacggccg
  841 gagcccgtct gccgtcgccg ccgccgcggc cgccgtcttc caccaacaac atggcggaca
  901 acaaagccct ggcctggcag cgctgcagag cggcggcggc gggggcctgg agccctacgc
  961 ggggccccag cagaactctc acgaccacgg cttccccaac caccagtaca actcctacta
1021 ccccaaccgc agcgcctacc ccccgcccgc cccggcctac gcgctgagct ccccgagagg
1081 tggcactccg ggctccggcg cggcggcggc tgccggctcc aagccgcctc cctcctccag
1141 cgcctccgcc tcctcgtcgt cttcgtcctt cgctcagcag cgcttcgggg ccatgggggg
1201 aggcggcccc tccgcggccg gcgggggaac tccccagccc accgccaccc ccaccctcaa
1261 ccaactgctc acgtcgccca gctcggcccg gggctaccag ggctaccccg ggggcgacta
1321 cagtggcggg ccccaggacg ggggcgccgg caagggcccg gcggacatgg cctcgcagtg
1381 ttggggggct gcggcggcgg cagctgcggc ggcggccgcc tcgggagggg cccaacaaag
1441 gagccaccac gcgcccatga gccccgggag cagcggcggc ggggggcagc cgctcgcccg
1501 gacccctcag ccatccagtc caatggatca gatgggcaag atgagacctc agccatatgg
1561 cgggactaac ccatactcgc agcaacaggg acctccgtca ggaccgcagc aaggacatgg
1621 gtacccaggg cagccatacg ggtcccagac cccgcagcgg tacccgatga ccatgcaggg
1681 ccgggcgcag agtgccatgg gcggcctctc ttatacacag cagattcctc cttatggaca
1741 acaaggcccc agcgggtatg gtcaacaggg ccagactcca tattacaacc agcaaagtcc
1801 tcaccctcag cagcagcagc caccctactc ccagcaacca ccgtcccaga cccctcatgc
1861 ccaaccttcg tatcagcagc agccacagtc tcaaccacca cagctccagt cctctcagcc
1921 tccatactcc cagcagccat cccagcctcc acatcagcag tccccggctc catacccctc
1981 ccagcagtcg acgacacagc agcaccccca gagccagccc ccctactcac agccacaggc
2041 tcagtctcct taccagcagc agcaacctca gcagccagca ccctcgacgc tctcccagca
2101 ggctgcgtat cctcagcccc agtctcagca gtcccagcaa actgcctatt cccagcagcg
2161 cttccctcca ccgcaggagc tatctcaaga ttcatttggg tctcaggcat cctcagcccc
2221 ctcaatgacc tccagtaagg gagggcaaga agatatgaac ctgagccttc agtcaagacc
2281 ctccagcttg cctgatctat ctggttcaat agatgacctc cccatgggga cagaaggagc
2341 tctgagtcct ggagtgagca catcagggat ttccagcagc caaggagagc agagtaatcc
2401 agctcagtct cctttctctc ctcatacctc ccctcacctg cctggcatcc gaggcccttc
2461 cccgtcccct gttggctctc ccgccagtgt tgctcagtct cgctcaggac cactctcgcc
2521 tgctgcagtg ccaggcaacc agatgccacc tcggccaccc agtggccagt cggacagcat
2581 catgcatcct tccatgaacc aatcaagcat tgcccaagat cgaggttata tgcagaggaa
2641 cccccagatg ccccagtaca gttcccccca gcccggctca gccttatctc cgcgtcagcc
2701 ttccggagga cagatacaca caggcatggg ctcctaccag cagaactcca tggggagcta
2761 tggtccccag gggggtcagt atggcccaca aggtggctac cccaggcagc caaactataa
2821 tgccttgccc aatgccaact accccagtgc aggcatggct ggaggcataa accccatggg
2881 tgccggaggt caaatgcatg gacagcctgg catcccacct tatggcacac tccctccagg
2941 gaggatgagt cacgcctcca tgggcaaccg gccttatggc cctaacatgg ccaatatgcc
3001 acctcaggtt gggtcaggga tgtgtccccc accagggggc atgaaccgga aaacccaaga
3061 aactgctgtc gccatgcatg ttgctgccaa ctctatccaa aacaggccgc caggctaccc
3121 caatatgaat caagggggca tgatgggaac tggacctcct tatggacaag ggattaatag
3181 tatggctggc atgatcaacc ctcagggacc cccatattcc atgggtggaa ccatggccaa
3241 caattctgca gggatggcag ccagcccaga gatgatgggc cttggggatg taaagttaac
3301 tccagccacc aaaatgaaca acaaggcaga tgggacaccc aagacagaat ccaaatccaa
3361 gaaatccagt tcttctacta caaccaatga gaagatcacc aagttgtatg agctgggtgg
3421 tgagcctgag aggaagatgt gggtggaccg ttatctggcc ttcactgagg agaaggccat
3481 gggcatgaca aatctgcctg ctgtgggtag gaaacctctg gacctctatc gcctctatgt
3541 gtctgtgaag gagattggtg gattgactca ggtcaacaag aacaaaaaat ggcgggaact
3601 tgcaaccaac ctcaatgtgg gcacatcaag cagtgctgcc agctccttga aaaagcagta
3661 tatccagtgt ctctatgcct ttgaatgcaa gattgaacgg ggagaagacc ctcccccaga
3721 catctttgca gctgctgatt ccaagaagtc ccagcccaag atccagcctc cctctcctgc
3781 gggatcagga tctatgcagg ggccccagac tccccagtca accagcagtt ccatggcaga
3841 aggaggagac ttaaagccac caactccagc atccacacca cacagtcaga tccccccatt
3901 gccaggcatg agcaggagca attcagttgg gatccaggat gcctttaatg atggaagtga
3961 ctccacattc cagaagcgga attccatgac tccaaaccct gggtatcagc ccagtatgaa
4021 tacctctgac atgatggggc gcatgtccta tgagccaaat aaggatcctt atggcagcat
4081 gaggaaagct ccagggagtg atcccttcat gtcctcaggg cagggcccca acggcgggat
4141 gggtgacccc tacagtcgtg ctgccggccc tgggctagga aatgtggcga tgggaccacg
4201 acagcactat ccctatggag gtccttatga cagagtgagg acggagcctg gaatagggcc
4261 tgagggaaac atgagcactg gggccccaca gccgaatctc atgccttcca acccagactc
4321 ggggatgtat tctcctagcc gctacccccc gcagcagcag cagcagcagc agcaacgaca
4381 tgattcctat ggcaatcagt tctccaccca aggcacccct tctggcagcc ccttccccag
4441 ccagcagact acaatgtatc aacagcaaca gcagaattac aagcggccaa tggatggcac
4501 atatggccct cctgccaagc ggcacgaagg ggagatgtac agcgtgccat acagcactgg
4561 gcaggggcag cctcagcagc agcagttgcc cccagcccag ccccagcctg ccagccagca
4621 acaagctgcc cagccttccc ctcagcaaga tgtatacaac cagtatggca atgcctatcc
4681 tgccactgcc acagctgcta ctgagcgccg accagcaggc ggcccccaga accaatttcc
4741 attccagttt ggccgagacc gtgtctctgc accccctggc accaatgccc agcaaaacat
4801 gccaccacaa atgatgggcg gccccataca ggcatcagct gaggttgctc agcaaggcac
4861 catgtggcag gggcgtaatg acatgaccta taattatgcc aacaggcaga gcacgggctc
4921 tgccccccag ggccccgcct atcatggcgt gaaccgaaca gatgaaatgc tgcacacaga
4981 tcagagggcc aaccacgaag gctcgtggcc ttcccatggc acacgccagc ccccatatgg
5041 tccctctgcc cctgtgcccc ccatgacaag gccccctcca tctaactacc agcccccacc
5101 aagcatgcag aatcacattc ctcaggtatc cagccctgct cccctgcccc ggccaatgga
5161 gaaccgcacc tctcctagca agtctccatt cctgcactct gggatgaaaa tgcagaaggc
5221 aggtccccca gtacctgcct cgcacatagc acctgcccct gtgcagcccc ccatgattcg
5281 gcgggatatc accttcccac ctggctctgt tgaagccaca cagcctgtgt tgaagcagag
5341 gaggcggctc acaatgaaag acattggaac cccggaggca tggcgggtaa tgatgtccct
5401 caagtctggt ctcctggcag agagcacatg ggcattagat accatcaaca tcctgctgta
5461 tgatgacaac agcatcatga ccttcaacct cagtcagctc ccagggttgc tagagctcct
5521 tgtagaatat ttccgacgat gcctgattga gatctttggc attttaaagg agtatgaggt
5581 gggtgaccca ggacagagaa cgctactgga tcctgggagg ttcagcaagg tgtctagtcc
5641 agctcccatg gagggtgggg aagaagaaga agaacttcta ggtcctaaac tagaagagga
5701 agaagaagag gaagtagttg aaaatgatga ggagatagcc ttttcaggca aggacaagcc
5761 agcttcagag aatagtgagg agaagctgat cagtaagttt gacaagcttc cagtaaagat
5821 cgtacagaag aatgatccat ttgtggtgga ctgctcagat aagcttgggc gtgtgcagga
5881 gtttgacagt ggcctgctgc actggcggat tggtgggggg gacaccactg agcatatcca
5941 gacccacttc gagagcaaga cagagctgct gccttcccgg cctcacgcac cctgcccacc
6001 agcccctcgg aagcatgtga caacagcaga gggtacacca gggacaacag accaggaggg
6061 gcccccacct gatggacctc cagaaaaacg gatcacagcc actatggatg acatgttgtc
6121 tactcggtct agcaccttga ccgaggatgg agctaagagt tcagaggcca tcaaggagag
6181 cagcaagttt ccatttggca ttagcccagc acagagccac cggaacatca agatcctaga
6241 ggacgaaccc cacagtaagg atgagacccc actgtgtacc cttctggact ggcaggattc
6301 tcttgccaag cgctgcgtct gtgtgtccaa taccattcga agcctgtcat ttgtgccagg
6361 caatgacttt gagatgtcca aacacccagg gctgctgctc atcctgggca agctgatcct
6421 gctgcaccac aagcacccag aacggaagca ggcaccacta acttatgaaa aggaggagga
6481 acaggaccaa ggggtgagct gcaacaaagt ggagtggtgg tgggactgct tggagatgct
6541 ccgggaaaac accttggtta cactcgccaa catctcgggg cagttggacc tatctccata
6601 ccccgagagc atttgcctgc ctgtcctgga cggactccta cactgggcag tttgcccttc
6661 agctgaagcc caggacccct tttccaccct gggccccaat gccgtccttt ccccgcagag
6721 actggtcttg gaaaccctca gcaaactcag catccaggac aacaatgtgg acctgattct
6781 ggccacaccc cccttcagcc gcctggagaa gttgtatagc actatggtgc gcttcctcag
6841 tgaccgaaag aacccggtgt gccgggagat ggctgtggta ctgctggcca acctggctca
6901 gggggacagc ctggcagctc gtgccattgc agtgcagaag ggcagtatcg gcaacctcct
6961 gggcttccta gaggacagcc ttgccgccac acagttccag cagagccagg ccagcctcct
7021 ccacatgcag aacccaccct ttgagccaac tagtgtggac atgatgcggc gggctgcccg
7081 cgcgctgctt gccttggcca aggtggacga gaaccactca gagtttactc tgtacgaatc
7141 acggctgttg gacatctcgg tatcaccgtt gatgaactca ttggtttcac aagtcatttg
7201 tgatgtactg tttttgattg gccagtcatg acagccgtgg gacacctccc ccccccgtgt
7261 gtgtgtgcgt gtgtggagaa cttagaaact gactgttgcc ctttatttat gcaaaaccac
7321 ctcagaatcc agtttaccct gtgctgtcca gcttctccct tgggaaaaag tctctcctgt
7381 ttctctctcc tccttccacc tcccctccct ccatcacctc acgcctttct gttccttgtc
7441 ctcaccttac tcccctcagg accctacccc accctctttg aaaagacaaa gctctgccta
7501 catagaagac tttttttatt ttaaccaaag ttactgttgt ttacagtgag tttggggaaa
7561 aaaaataaaa taaaaatggc tttcccagtc cttgcatcaa cgggatgcca catttcataa
7621 ctgtttttaa tggtaaaaaa aaaaaaaaaa aatacaaaaa aaaattctga aggacaaaaa
7681 aggtgactgc tgaactgtgt gtggtttatt gttgtacatt cacaatcttg caggagccaa
7741 gaagttcgca gttgtgaaca gaccctgttc actggagagg cctgtgcagt agagtgtaga
7801 ccctttcatg tactgtactg tacacctgat actgtaaaca tactgtaata ataatgtctc
7861 acatggaaac agaaaacgct gggtcagcag caagctgtag tttttaaaaa tgtttttagt
7921 taaacgttga ggagaaaaaa aaaaaaggct tttcccccaa agtatcatgt gtgaacctac
7981 aacaccctga cctctttctc tcctccttga ttgtatgaat aaccctgaga tcacctctta
8041 gaactggttt taacctttag ctgcagcggc tacgctgcca cgtgtgtata tatatgacgt
8101 tgtacattgc acataccctt ggatccccac agtttggtcc tcctcccagc taccccttta
8161 tagtatgacg agttaacaag ttggtgacct gcacaaagcg agacacagct atttaatctc
8221 ttgccagata tcgcccctct tggtgcgatg ctgtacaggt ctctgtaaaa agtccttgct
8281 gtctcagcag ccaatcaact tatagtttat ttttttctgg gtttttgttt tgttttgttt
8341 tctttctaat cgaggtgtga aaaagttcta ggttcagttg aagttctgat gaagaaacac
8401 aattgagatt ttttcagtga taaaatctgc atatttgtat ttcaacaatg tagctaaaac
8461 ttgatgtaaa ttcctccttt ttttcctttt ttggcttaat gaatatcatt tattcagtat
8521 gaaatcttta tactatatgt tccacgtgtt aagaataaat gtacattaaa tcttggtaag
8581 acttt
AT-rich interactive domain-containing protein 1A (ARID1A) isoform b
(SEQ ID NO: 11)
      1 maaqvapaaa sslgnppppp pselkkaeqq qreeaggeaa aaaaaergem kaaagqeseg
    61 pavgppqplg kelqdgaesn gggggggags gggpgaepdl knsngnagpr palnnnltep
  121 pggggggssd gvgapphsaa aalpppaygf gqpygrspsa vaaaaaavfh qqhggqqspg
  181 laalqsgggg glepyagpqq nshdhgfpnh qynsyypnrs aypppapaya lssprggtpg
  241 sgaaaaagsk pppsssasas sssssfaqqr fgamggggps aagggtpqpt atptlnqllt
  301 spssargyqg ypggdysggp qdggagkgpa dmasqcwgaa aaaaaaaaas ggaqqrshha
  361 pmspgssggg gqplartpqp sspmdqmgkm rpqpyggtnp ysqqqgppsg pqqghgypgq
  421 pygsqtpqry pmtmqgraqs amgglsytqq ippygqqgps gygqqgqtpy ynqqsphpqq
  481 qqppysqqpp sqtphaqpsy qqqpqsqppq lqssqppysq qpsqpphqqs papypsqqst
  541 tqqhpqsqpp ysqpqaqspy qqqqpqqpap stlsqqaayp qpqsqqsqqt aysqqrfppp
  601 qelsqdsfgs qassapsmts skggqedmnl slqsrpsslp dlsgsiddlp mgtegalspg
  661 vstsgisssq geqsnpaqsp fsphtsphlp girgpspspv gspasvaqsr sgplspaavp
  721 gnqmpprpps gqsdsimhps mnqssiaqdr gymqrnpqmp qysspqpgsa lsprqpsggq
  781 ihtgmgsyqq nsmgsygpqg gqygpqggyp rqpnynalpn anypsagmag ginpmgaggq
  841 mhgqpgippy gtlppgrmsh asmgnrpygp nmanmppqvg sgmcpppggm nrktqetava
  901 mhvaansiqn rppgypnmnq ggmmgtgppy gqginsmagm inpqgppysm ggtmannsag
  961 maaspemmgl gdvkltpatk mnnkadgtpk teskskksss stttnekitk lyelggeper
1021 kmwvdrylaf teekamgmtn lpavgrkpld lyrlyvsvke iggltqvnkn kkwrelatnl
1081 nvgtsssaas slkkqyiqcl yafeckierg edpppdifaa adskksqpki qppspagsgs
1141 mqgpqtpqst sssmaeggdl kpptpastph sqipplpgms rsnsvgiqda fndgsdstfq
1201 krnsmtpnpg yqpsmntsdm mgrmsyepnk dpygsmrkap gsdpfmssgq gpnggmgdpy
1261 sraagpglgn vamgprqhyp yggpydrvrt epgigpegnm stgapqpnlm psnpdsgmys
1321 psryppqqqq qqqqrhdsyg nqfstqgtps gspfpsqqtt myqqqqqvss paplprpmen
1381 rtspskspfl hsgmkmqkag ppvpashiap apvqppmirr ditfppgsve atqpvlkqrr
1441 rltmkdigtp eawrvmmslk sgllaestwa ldtinillyd dnsimtfnls qlpgllellv
1501 eyfrrcliei fgilkeyevg dpgqrtlldp grfskvsspa pmeggeeeee llgpkleeee
1561 eeevvendee iafsgkdkpa senseeklis kfdklpvkiv qkndpfvvdc sdklgrvqef
1621 dsgllhwrig ggdttehiqt hfesktellp srphapcppa prkhvttaeg tpgttdqegp
1681 ppdgppekri tatmddmlst rsstltedga ksseaikess kfpfgispaq shrnikiled
1741 ephskdetpl ctlldwqdsl akrcvcvsnt irslsfvpgn dfemskhpgl llilgklill
1801 hhkhperkqa pltyekeeeq dqgvscnkve wwwdclemlr entlvtlani sgqldlspyp
1861 esiclpvldg llhwavcpsa eaqdpfstlg pnavlspqrl vletlsklsi qdnnvdlila
1921 tppfsrlekl ystmvrflsd rknpvcrema vvllanlaqg dslaaraiav qkgsignllg
1981 fledslaatq fqqsqasllh mqnppfepts vdmmrraara llalakvden hseftlyesr
2041 lldisvsplm nslvsqvicd vlfligqs
Homo sapiens AT rich interactive domain 1A (SWI-like) (ARID1A),
transcript variant 2, mRNA
(SEQ ID NO: 12)
      1 cagaaagcgg agagtcacag cggggccagg ccctggggag cggagcctcc accgcccccc
    61 tcattcccag gcaagggctt ggggggaatg agccgggaga gccgggtccc gagcctacag
  121 agccgggagc agctgagccg ccggcgcctc ggccgccgcc gccgcctcct cctcctccgc
  181 cgccgccagc ccggagcctg agccggcggg gcggggggga gaggagcgag cgcagcgcag
  241 cagcggagcc ccgcgaggcc cgcccgggcg ggtggggagg gcagcccggg ggactgggcc
  301 ccggggcggg gtgggagggg gggagaagac gaagacaggg ccgggtctct ccgcggacga
  361 gacagcgggg atcatggccg cgcaggtcgc ccccgccgcc gccagcagcc tgggcaaccc
  421 gccgccgccg ccgccctcgg agctgaagaa agccgagcag cagcagcggg aggaggcggg
  481 gggcgaggcg gcggcggcgg cagcggccga gcgcggggaa atgaaggcag ccgccgggca
  541 ggaaagcgag ggccccgccg tggggccgcc gcagccgctg ggaaaggagc tgcaggacgg
  601 ggccgagagc aatgggggtg gcggcggcgg cggagccggc agcggcggcg ggcccggcgc
  661 ggagccggac ctgaagaact cgaacgggaa cgcgggccct aggcccgccc tgaacaataa
  721 cctcacggag ccgcccggcg gcggcggtgg cggcagcagc gatggggtgg gggcgcctcc
  781 tcactcagcc gcggccgcct tgccgccccc agcctacggc ttcgggcaac cctacggccg
  841 gagcccgtct gccgtcgccg ccgccgcggc cgccgtcttc caccaacaac atggcggaca
  901 acaaagccct ggcctggcag cgctgcagag cggcggcggc gggggcctgg agccctacgc
  961 ggggccccag cagaactctc acgaccacgg cttccccaac caccagtaca actcctacta
1021 ccccaaccgc agcgcctacc ccccgcccgc cccggcctac gcgctgagct ccccgagagg
1081 tggcactccg ggctccggcg cggcggcggc tgccggctcc aagccgcctc cctcctccag
1141 cgcctccgcc tcctcgtcgt cttcgtcctt cgctcagcag cgcttcgggg ccatgggggg
1201 aggcggcccc tccgcggccg gcgggggaac tccccagccc accgccaccc ccaccctcaa
1261 ccaactgctc acgtcgccca gctcggcccg gggctaccag ggctaccccg ggggcgacta
1321 cagtggcggg ccccaggacg ggggcgccgg caagggcccg gcggacatgg cctcgcagtg
1381 ttggggggct gcggcggcgg cagctgcggc ggcggccgcc tcgggagggg cccaacaaag
1441 gagccaccac gcgcccatga gccccgggag cagcggcggc ggggggcagc cgctcgcccg
1501 gacccctcag ccatccagtc caatggatca gatgggcaag atgagacctc agccatatgg
1561 cgggactaac ccatactcgc agcaacaggg acctccgtca ggaccgcagc aaggacatgg
1621 gtacccaggg cagccatacg ggtcccagac cccgcagcgg tacccgatga ccatgcaggg
1681 ccgggcgcag agtgccatgg gcggcctctc ttatacacag cagattcctc cttatggaca
1741 acaaggcccc agcgggtatg gtcaacaggg ccagactcca tattacaacc agcaaagtcc
1801 tcaccctcag cagcagcagc caccctactc ccagcaacca ccgtcccaga cccctcatgc
1861 ccaaccttcg tatcagcagc agccacagtc tcaaccacca cagctccagt cctctcagcc
1921 tccatactcc cagcagccat cccagcctcc acatcagcag tccccggctc catacccctc
1981 ccagcagtcg acgacacagc agcaccccca gagccagccc ccctactcac agccacaggc
2041 tcagtctcct taccagcagc agcaacctca gcagccagca ccctcgacgc tctcccagca
2101 ggctgcgtat cctcagcccc agtctcagca gtcccagcaa actgcctatt cccagcagcg
2161 cttccctcca ccgcaggagc tatctcaaga ttcatttggg tctcaggcat cctcagcccc
2221 ctcaatgacc tccagtaagg gagggcaaga agatatgaac ctgagccttc agtcaagacc
2281 ctccagcttg cctgatctat ctggttcaat agatgacctc cccatgggga cagaaggagc
2341 tctgagtcct ggagtgagca catcagggat ttccagcagc caaggagagc agagtaatcc
2401 agctcagtct cctttctctc ctcatacctc ccctcacctg cctggcatcc gaggcccttc
2461 cccgtcccct gttggctctc ccgccagtgt tgctcagtct cgctcaggac cactctcgcc
2521 tgctgcagtg ccaggcaacc agatgccacc tcggccaccc agtggccagt cggacagcat
2581 catgcatcct tccatgaacc aatcaagcat tgcccaagat cgaggttata tgcagaggaa
2641 cccccagatg ccccagtaca gttcccccca gcccggctca gccttatctc cgcgtcagcc
2701 ttccggagga cagatacaca caggcatggg ctcctaccag cagaactcca tggggagcta
2761 tggtccccag gggggtcagt atggcccaca aggtggctac cccaggcagc caaactataa
2821 tgccttgccc aatgccaact accccagtgc aggcatggct ggaggcataa accccatggg
2881 tgccggaggt caaatgcatg gacagcctgg catcccacct tatggcacac tccctccagg
2941 gaggatgagt cacgcctcca tgggcaaccg gccttatggc cctaacatgg ccaatatgcc
3001 acctcaggtt gggtcaggga tgtgtccccc accagggggc atgaaccgga aaacccaaga
3061 aactgctgtc gccatgcatg ttgctgccaa ctctatccaa aacaggccgc caggctaccc
3121 caatatgaat caagggggca tgatgggaac tggacctcct tatggacaag ggattaatag
3181 tatggctggc atgatcaacc ctcagggacc cccatattcc atgggtggaa ccatggccaa
3241 caattctgca gggatggcag ccagcccaga gatgatgggc cttggggatg taaagttaac
3301 tccagccacc aaaatgaaca acaaggcaga tgggacaccc aagacagaat ccaaatccaa
3361 gaaatccagt tcttctacta caaccaatga gaagatcacc aagttgtatg agctgggtgg
3421 tgagcctgag aggaagatgt gggtggaccg ttatctggcc ttcactgagg agaaggccat
3481 gggcatgaca aatctgcctg ctgtgggtag gaaacctctg gacctctatc gcctctatgt
3541 gtctgtgaag gagattggtg gattgactca ggtcaacaag aacaaaaaat ggcgggaact
3601 tgcaaccaac ctcaatgtgg gcacatcaag cagtgctgcc agctccttga aaaagcagta
3661 tatccagtgt ctctatgcct ttgaatgcaa gattgaacgg ggagaagacc ctcccccaga
3721 catctttgca gctgctgatt ccaagaagtc ccagcccaag atccagcctc cctctcctgc
3781 gggatcagga tctatgcagg ggccccagac tccccagtca accagcagtt ccatggcaga
3841 aggaggagac ttaaagccac caactccagc atccacacca cacagtcaga tccccccatt
3901 gccaggcatg agcaggagca attcagttgg gatccaggat gcctttaatg atggaagtga
3961 ctccacattc cagaagcgga attccatgac tccaaaccct gggtatcagc ccagtatgaa
4021 tacctctgac atgatggggc gcatgtccta tgagccaaat aaggatcctt atggcagcat
4081 gaggaaagct ccagggagtg atcccttcat gtcctcaggg cagggcccca acggcgggat
4141 gggtgacccc tacagtcgtg ctgccggccc tgggctagga aatgtggcga tgggaccacg
4201 acagcactat ccctatggag gtccttatga cagagtgagg acggagcctg gaatagggcc
4261 tgagggaaac atgagcactg gggccccaca gccgaatctc atgccttcca acccagactc
4321 ggggatgtat tctcctagcc gctacccccc gcagcagcag cagcagcagc agcaacgaca
4381 tgattcctat ggcaatcagt tctccaccca aggcacccct tctggcagcc ccttccccag
4441 ccagcagact acaatgtatc aacagcaaca gcaggtatcc agccctgctc ccctgccccg
4501 gccaatggag aaccgcacct ctcctagcaa gtctccattc ctgcactctg ggatgaaaat
4561 gcagaaggca ggtcccccag tacctgcctc gcacatagca cctgcccctg tgcagccccc
4621 catgattcgg cgggatatca ccttcccacc tggctctgtt gaagccacac agcctgtgtt
4681 gaagcagagg aggcggctca caatgaaaga cattggaacc ccggaggcat ggcgggtaat
4741 gatgtccctc aagtctggtc tcctggcaga gagcacatgg gcattagata ccatcaacat
4801 cctgctgtat gatgacaaca gcatcatgac cttcaacctc agtcagctcc cagggttgct
4861 agagctcctt gtagaatatt tccgacgatg cctgattgag atctttggca ttttaaagga
4921 gtatgaggtg ggtgacccag gacagagaac gctactggat cctgggaggt tcagcaaggt
4981 gtctagtcca gctcccatgg agggtgggga agaagaagaa gaacttctag gtcctaaact
5041 agaagaggaa gaagaagagg aagtagttga aaatgatgag gagatagcct tttcaggcaa
5101 ggacaagcca gcttcagaga atagtgagga gaagctgatc agtaagtttg acaagcttcc
5161 agtaaagatc gtacagaaga atgatccatt tgtggtggac tgctcagata agcttgggcg
5221 tgtgcaggag tttgacagtg gcctgctgca ctggcggatt ggtggggggg acaccactga
5281 gcatatccag acccacttcg agagcaagac agagctgctg ccttcccggc ctcacgcacc
5341 ctgcccacca gcccctcgga agcatgtgac aacagcagag ggtacaccag ggacaacaga
5401 ccaggagggg cccccacctg atggacctcc agaaaaacgg atcacagcca ctatggatga
5461 catgttgtct actcggtcta gcaccttgac cgaggatgga gctaagagtt cagaggccat
5521 caaggagagc agcaagtttc catttggcat tagcccagca cagagccacc ggaacatcaa
5581 gatcctagag gacgaacccc acagtaagga tgagacccca ctgtgtaccc ttctggactg
5641 gcaggattct cttgccaagc gctgcgtctg tgtgtccaat accattcgaa gcctgtcatt
5701 tgtgccaggc aatgactttg agatgtccaa acacccaggg ctgctgctca tcctgggcaa
5761 gctgatcctg ctgcaccaca agcacccaga acggaagcag gcaccactaa cttatgaaaa
5821 ggaggaggaa caggaccaag gggtgagctg caacaaagtg gagtggtggt gggactgctt
5881 ggagatgctc cgggaaaaca ccttggttac actcgccaac atctcggggc agttggacct
5941 atctccatac cccgagagca tttgcctgcc tgtcctggac ggactcctac actgggcagt
6001 ttgcccttca gctgaagccc aggacccctt ttccaccctg ggccccaatg ccgtcctttc
6061 cccgcagaga ctggtcttgg aaaccctcag caaactcagc atccaggaca acaatgtgga
6121 cctgattctg gccacacccc ccttcagccg cctggagaag ttgtatagca ctatggtgcg
6181 cttcctcagt gaccgaaaga acccggtgtg ccgggagatg gctgtggtac tgctggccaa
6241 cctggctcag ggggacagcc tggcagctcg tgccattgca gtgcagaagg gcagtatcgg
6301 caacctcctg ggcttcctag aggacagcct tgccgccaca cagttccagc agagccaggc
6361 cagcctcctc cacatgcaga acccaccctt tgagccaact agtgtggaca tgatgcggcg
6421 ggctgcccgc gcgctgcttg ccttggccaa ggtggacgag aaccactcag agtttactct
6481 gtacgaatca cggctgttgg acatctcggt atcaccgttg atgaactcat tggtttcaca
6541 agtcatttgt gatgtactgt ttttgattgg ccagtcatga cagccgtggg acacctcccc
6601 cccccgtgtg tgtgtgcgtg tgtggagaac ttagaaactg actgttgccc tttatttatg
6661 caaaaccacc tcagaatcca gtttaccctg tgctgtccag cttctccctt gggaaaaagt
6721 ctctcctgtt tctctctcct ccttccacct cccctccctc catcacctca cgcctttctg
6781 ttccttgtcc tcaccttact cccctcagga ccctacccca ccctctttga aaagacaaag
6841 ctctgcctac atagaagact ttttttattt taaccaaagt tactgttgtt tacagtgagt
6901 ttggggaaaa aaaataaaat aaaaatggct ttcccagtcc ttgcatcaac gggatgccac
6961 atttcataac tgtttttaat ggtaaaaaaa aaaaaaaaaa atacaaaaaa aaattctgaa
7021 ggacaaaaaa ggtgactgct gaactgtgtg tggtttattg ttgtacattc acaatcttgc
7081 aggagccaag aagttcgcag ttgtgaacag accctgttca ctggagaggc ctgtgcagta
7141 gagtgtagac cctttcatgt actgtactgt acacctgata ctgtaaacat actgtaataa
7201 taatgtctca catggaaaca gaaaacgctg ggtcagcagc aagctgtagt ttttaaaaat
7261 gtttttagtt aaacgttgag gagaaaaaaa aaaaaggctt ttcccccaaa gtatcatgtg
7321 tgaacctaca acaccctgac ctctttctct cctccttgat tgtatgaata accctgagat
7381 cacctcttag aactggtttt aacctttagc tgcagcggct acgctgccac gtgtgtatat
7441 atatgacgtt gtacattgca catacccttg gatccccaca gtttggtcct cctcccagct
7501 acccctttat agtatgacga gttaacaagt tggtgacctg cacaaagcga gacacagcta
7561 tttaatctct tgccagatat cgcccctctt ggtgcgatgc tgtacaggtc tctgtaaaaa
7621 gtccttgctg tctcagcagc caatcaactt atagtttatt tttttctggg tttttgtttt
7681 gttttgtttt ctttctaatc gaggtgtgaa aaagttctag gttcagttga agttctgatg
7741 aagaaacaca attgagattt tttcagtgat aaaatctgca tatttgtatt tcaacaatgt
7801 agctaaaact tgatgtaaat tcctcctttt tttccttttt tggcttaatg aatatcattt
7861 attcagtatg aaatctttat actatatgtt ccacgtgtta agaataaatg tacattaaat
7921 cttggtaaga cttt
```

The term “inducing differentiation” used herein refers to causing an immature or stem-like cell to develop into a more differentiated or terminally differentiated cell.

According to the methods of the disclosure, a “normal” cell may be used as a basis of comparison for one or more characteristics of a cancer cell, including expression and/or function of SNF5, ATRX, and/or ARID1A. As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell expresses a comparable amount of EZH2 as a cancer cell. Preferably a normal cell contains a wild type sequence for a SNF5, ATRX, and/or ARID1A gene, expresses a SNF5, ATRX, and/or ARID1A transcript without mutations, and expresses a SNF5, ATRX, and/or ARID1A protein without mutations that retains all functions a normal activity levels.

As used herein, “contacting a cell” refers to a condition in which a compound or other composition of matter is in direct contact with a cell, or is close enough to induce a desired biological effect in a cell.

As used herein, “treating” or “treat” describes the management and care of a subject for the purpose of combating a disease, condition, or disorder and includes the administration of an EZH2 inhibitor of the disclosure, or a pharmaceutically acceptable salt, prodrug, metabolite, polymorph or solvate thereof, to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

An effective amount of an EZH2 inhibitor of the disclosure, or a pharmaceutically acceptable salt, prodrug, metabolite, polymorph or solvate thereof, is not significantly cytotoxic to normal cells. For example, a therapeutically effective amount of an EZH2 inhibitor of the disclosure is not significantly cytotoxic to normal cells if administration of the EZH2 inhibitor of the disclosure in a therapeutically effective amount does not induce cell death in greater than 10% of normal cells. A therapeutically effective amount of an EZH2 inhibitor of the disclosure does not significantly affect the viability of normal cells if administration of the compound in a therapeutically effective amount does not induce cell death in greater than 10% of normal cells.

Contacting a cell with an EZH2 inhibitor of the disclosure, or a pharmaceutically acceptable salt, prodrug, metabolite, polymorph or solvate thereof, can inhibit EZH2 activity selectively in cancer cells. Administering to a subject in need thereof an EZH2 inhibitor of the disclosure, or a pharmaceutically acceptable salt, prodrug, metabolite, polymorph or solvate thereof, can inhibit EZH2 activity selectively in cancer cells.

### EZH2 Inhibitors

EZH2 inhibitors of the disclosure comprise tazemetostat (EPZ-6438):

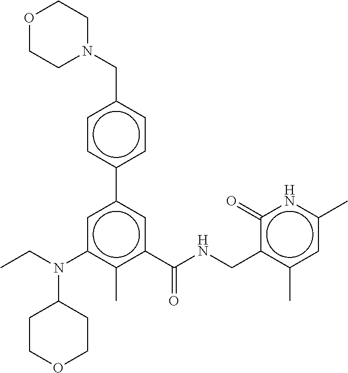

or a pharmaceutically acceptable salt thereof

Tazemetostat is also described in U.S. Pat. Nos. 8,410,088, 8,765,732, and 9,090,562 (the contents of which are each incorporated herein in their entireties).

Tazemetostat or a pharmaceutically acceptable salt thereof, as described herein, is potent in targeting both WT and mutant EZH2. Tazemetostat is orally bioavailable and has high selectivity to EZH2 compared with other histone methyltransferases (i.e. >20,000 fold selectivity by Ki). Importantly, tazemetostat has targeted methyl mark inhibition that results in the killing of genetically defined cancer cells in vitro. Animal models have also shown sustained in vivo efficacy following inhibition of the target methyl mark. Clinical trial results described herein also demonstrate the safety and efficacy of tazemetostat.

In one embodiment, tazemetostat or a pharmaceutically acceptable salt thereof is administered to the subject at a dose of approximately 100 mg to approximately 3200 mg daily, such as about 100 mg BID to about 1600 mg BID (e.g., 100 mg BID, 200 mg BID, 400 mg BID, 800 mg BID, or 1600 mg BID), for treating a NHL. On one embodiment the dose is 800 mg BID.

EZH2 inhibitors of the disclosure may comprise, consist essentially of or consist of:

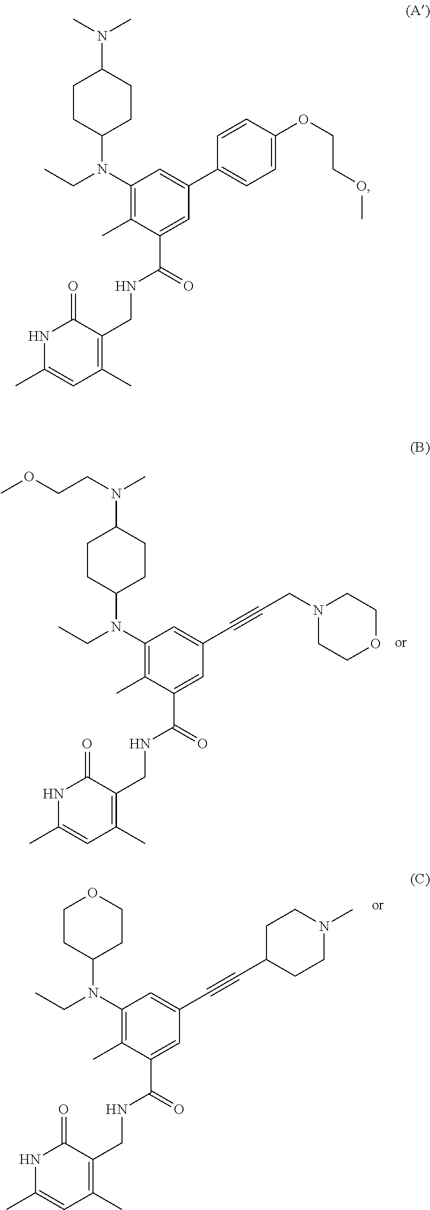

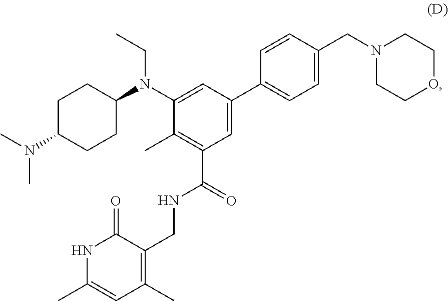 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]

or stereoisomers thereof or pharmaceutically acceptable salts and solvates thereof

EZH2 inhibitors of the disclosure may comprise, consist essentially of or consist of Compound E:

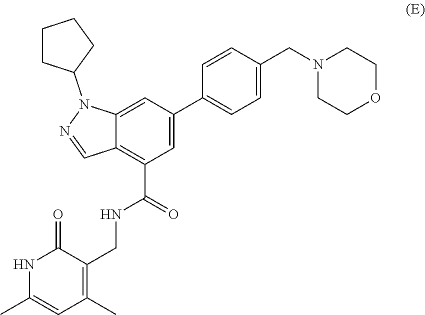 [SMILES: Cc1cc(C)c(CNC(=O)c2cc(-c3ccc(CN4CCOCC4)cc3)cc3c2cnn3C2CCCC2)c(=O)[nH]1]

or pharmaceutically acceptable salts thereof

EZH2 inhibitors of the disclosure may comprise, consist essentially of or consist of GSK-126, having the following formula:

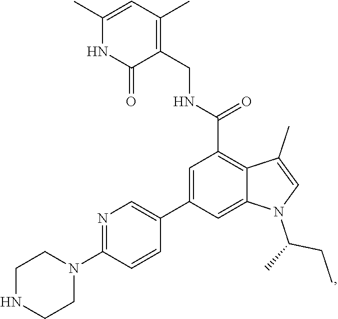 [SMILES: CC[C@H](C)n1cc(C)c2c(C(=O)NCc3c(C)cc(C)[nH]c3=O)cc(-c3ccc(N4CCNCC4)nc3)cc21]

stereoisomers thereof, or pharmaceutically acceptable salts or solvates thereof

EZH2 inhibitors of the disclosure may comprise, consist essentially of or consist of Compound F:

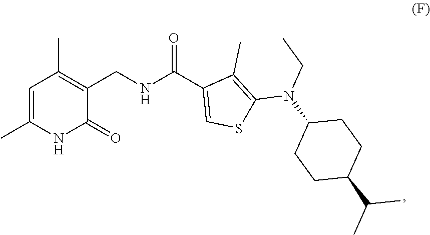 [SMILES: CCN(c1scc(C(=O)NCc2c(C)cc(C)[nH]c2=O)c1C)[C@H]1CC[C@H](C(C)C)CC1]

or stereoisomers thereof or pharmaceutically acceptable salts and solvates thereof

EZH2 inhibitors of the disclosure may comprise, consist essentially of or consist of any one of Compounds Ga-Gc:

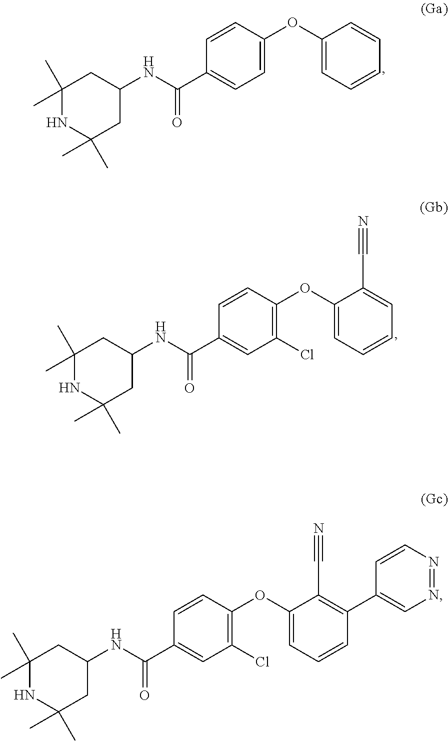 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

or a stereoisomer, pharmaceutically acceptable salt or solvate thereof.

EZH2 inhibitors of the disclosure may comprise, consist essentially of or consist of CPI-1205 or GSK343.

Additional suitable EZH2 inhibitors will be apparent to those skilled in the art. In some embodiments of the strategies, treatment modalities, methods, combinations, and compositions provided herein, the EZH2 inhibitor is an EZH2 inhibitor described in U.S. Pat. No. 8,536,179 (describing GSK-126 among other compounds and corresponding to WO 2011/140324), the entire contents of each of which are incorporated herein by reference.

In some embodiments of the strategies, treatment modalities, methods, combinations, and compositions provided herein, the EZH2 inhibitor is an EZH2 inhibitor described in PCT/US2014/015706, published as WO 2014/124418, in PCT/US2013/025639, published as WO 2013/120104, and in U.S. Ser. No. 14/839,273, published as US 2015/0368229, the entire contents of each of which are incorporated herein by reference

In one embodiment, the compound disclosed herein is the compound itself, i.e., the free base or “naked” molecule. In another embodiment, the compound is a salt thereof, e.g., a mono-HC1 or tri-HCl salt, mono-HBr or tri-HBr salt of the naked molecule.

Compounds disclosed herein that contain nitrogens can be converted to N-oxides by treatment with an oxidizing agent (e.g., 3-chloroperoxybenzoic acid (mCPBA) and/or hydrogen peroxides) to afford other compounds suitable for any methods disclosed herein. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds disclosed herein can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds disclosed herein may be depicted as different chiral isomers or geometric isomers. It should also be understood that when compounds have chiral isomeric or geometric isomeric forms, all isomeric forms are intended to be included in the scope of the disclosure, and the naming of the compounds does not exclude any isomeric forms.

Furthermore, the structures and other compounds discussed in this disclosure include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of keto-enol equilibria is between pyridin-2(1H)-ones and the corresponding pyridin-2-ols, as shown below.

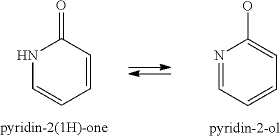 [SMILES: O=c1cccc[nH]1 ; Oc1ccccn1]

It is to be understood that the compounds disclosed herein may be depicted as different tautomers. It should also be understood that when compounds have tautomeric forms, all tautomeric forms are intended to be included in the scope of the disclosure, and the naming of the compounds does not exclude any tautomer form.

The compounds disclosed herein include the compounds themselves, as well as their salts and their solvates, if applicable. A salt, for example, can be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms. In the salt form, it is understood that the ratio of the compound to the cation or anion of the salt can be 1:1, or any ration other than 1:1, e.g., 3:1, 2:1, 1:2, or 1:3.

Additionally, the compounds disclosed herein, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure, and are substituted with various groups as described herein. For example, all of the compounds represented by Formula (I) are aryl- or heteroaryl-substituted benzene compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

### Pharmaceutical Formulations

The present disclosure also provides pharmaceutical compositions comprising at least one EZH2 inhibitor described herein in combination with at least one pharmaceutically acceptable excipient or carrier.

A “pharmaceutical composition” is a formulation containing the EZH2 inhibitors of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the disclosure includes both one and more than one such excipient. A pharmaceutical composition of the disclosure is formulated to be compatible with its intended route of administration. Examples of routes of administration include parenteral, (e.g., intravenous, intradermal, subcutaneous), and enteral routes (e.g., oral, buccal, sublingual, sublabial), as well as administration by inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Suitable formulations for enteral application, e.g., for oral administration, include, for example, tablets, capsules, time-release or sustained-release tablets and capsules, powders or granules (e.g., for formulating a solution or suspension that is orally administered), syrups, solutions, or suspensions. Liquid formulations for oral administration may include one or more diluent, e.g., water, which may, in some embodiments, be sterile. Such liquid formulations for oral administration may also include a stabilizer, an antibacterial agent, an antioxidant, a chelating agent, a buffer, an agent for the adjustment of tonicity, and agents to control appearance and taste, such as a sweetener and/or a flavoring agent. Powders from which a solution or suspension can be reconstituted before oral administration may contain similar agents.

A compound or pharmaceutical composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of an EZH2 inhibitor, composition, or pharmaceutical composition thereof effective to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer, including but not limited to, an INI1-deficient tumor.

For any EZH2 inhibitor of the disclosure, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Some embodiments provide pharmaceutical compositions, dosage forms, and/or methods of using such compositions or dosage forms, wherein an EZH2 inhibitor is formulated as an oral tablet, or as a suspension or solution. In some such embodiments, the EZH2 inhibitor may be formulated for administration at a dose of between 10 mg/kg/day and 1600 mg/kg/day. In some embodiments, the pharmaceutical composition, or dosage form may be administered to a subject at a dose of about 100, 200, 400, 800, or 1600 mg. In some embodiments, an EZH2 inhibitor may be formulated for administration at a dose of about 800 mg. Such formulation may comprise one or multiple dosage forms, e.g., a single tablet or capsule, or a plurality of tablets or capsules, or a certain amount of powder, solution, or suspension comprising the EZH2 inhibitor. In some embodiments, pharmaceutical compositions or dosage forms are provided in which an EZH2 inhibitor is formulated for administration once or twice per day (BID). In some embodiments, pharmaceutical compositions or dosage forms are provided in which an EZH2 inhibitor is formulated for administration at a dose of between 10 mg/kg/day and 1600 mg/kg/day BID. For example, in some embodiments, a pharmaceutical composition is provided that is suitable for administration of an EZH2 inhibitor at a dose of 800 mg BID.

In some embodiments, a pharmaceutical composition or dosage form comprising an EZH2 inhibitor is provided that is formulated for parenteral or enteral administration, for example, as an oral tablet, suspension, or solution, or as a solution or suspension for administration to the CSF by any route. In some such embodiments, the pharmaceutical composition or dosage form may be suitable for administration of the EZH2 inhibitor at a dose of between 10 mg/kg/day and 1600 mg/kg/day, e.g., at a dose of 10 mg/kg/day, 20 mg/kg/day, 25 mg/kg/day, 30 mg/kg/day, 40 mg/kg/day, 50 mg/kg/day, 60 mg/kg/day, 70 mg/kg/day, 75 mg/kg/day, 80 mg/kg/day, 90 mg/kg/day, 100 mg/kg/day, 200 mg/kg/day, 250 mg/kg/day, 300 mg/kg/day, 400 mg/kg/day, 500 mg/kg/day, 600 mg/kg/day, 700 mg/kg/day, 750 mg/kg/day, 800 mg/kg/day, 900 mg/kg/day, 1000 mg/kg/day, 1100 mg/kg/day, 1200 mg/kg/day, 1250 mg/kg/day, 1300 mg/kg/day, 1400 mg/kg/day, 1500 mg/kg/day, or 1600 mg/kg/day. For example, in some embodiments, a pharmaceutical composition or dosage form is provided that is suitable for administration of an EZH2 inhibitor at a dose of between 10 mg/kg/day and 1600 mg/kg/day BID. For example, EZH2 inhibitors of the disclosure may be administered at a dose of 800 mg BID.

In some embodiments, a pharmaceutical composition or dosage form comprising an EZH2 inhibitor is provided that is formulated for parenteral or enteral administration, for example, as an oral tablet, suspension, or solution. In some such embodiments, the pharmaceutical composition or dosage form may be suitable for administration of the EZH2 inhibitor at a dose of between 10 mg/m²/day and 1200 mg/m²/day, e.g., at a dose of 10 mg/m²/day, 20 mg/m²/day, 25 mg/m²/day, 30 mg/m²/day, 40 mg/m²/day, 50 mg/m²/day, 60 mg/m²/day, 70 mg/m²/day, 75 mg/m²/day, 80 mg/m²/day, 90 mg/m²/day, 100 mg/m²/day, 110 mg/m²/day, 120 mg/m2/day, 125 mg/m²/day, 130 mg/m²/day, 140 mg/m²/day, 150 mg/m²/day, 160 mg/m²/day, 170 mg/m²/day, 175 mg/m²/day, 180 mg/m²/day, 190 mg/m²/day, 200 mg/m²/day, 210 mg/m²/day, 220 mg/m²/day, 225 mg/m²/day, 230 mg/m²/day, 240 mg/m²/day, 250 mg/m²/day, 260 mg/m²/day, 270 mg/m²/day, 275 mg/m²/day, 280 mg/m²/day, 290 mg/m²/day, 300 mg/m²/day, 310 mg/m²/day, 320 mg/m²/day, 325 mg/m²/day, 330 mg/m²/day, 340 mg/m²/day, 350 mg/m²/day, 360 mg/m²/day, 370 mg/m²/day, 375 mg/m²/day, 380 mg/m²/day, 390 mg/m²/day, 400 mg/m²/day, 410 mg/m²/day, 420 mg/m²/day, 425 mg/m²/day, 430 mg/m²/day, 440 mg/m²/day, 450 mg/m²/day, 460 mg/m²/day, 470 mg/m²/day, 475 mg/m²/day, 480 mg/m²/day, 490 mg/m²/day, 500 mg/m²/day, 525 mg/m²/day, 550 mg/m²/day, 575 mg/m²/day, 600 mg/m²/day, 625 mg/m²/day, 650 mg/m²/day, 675 mg/m²/day, 700 mg/m²/day, 750 mg/m²/day, 800 mg/m²/day, 850 mg/m²/day, 900 mg/m²/day, or 1000 mg/m²/day. For example, in some embodiments, a pharmaceutical composition or dosage form is provided that is suitable for administration of an EZH2 inhibitor at a dose of between 10 mg/m²/day and 1200 mg/m²/day, e.g., between 100 and 300 mg/m²/day, between 200 and 300 mg/m²/day, between 200 and 400 mg/m²/day, between 250 and 500 mg/m²/day, between 150 and 400 mg/m²/day, between 150 and 300 mg/m²/day, between 300 and 600 mg/m²/day, between 350 and 400 mg/m²/day, between 350 and 700 mg/m²/day, or between 400 and 1200 mg/m²/day. For example, in some embodiments, a pharmaceutical composition or dosage form is provided that is suitable for administration of an EZH2 inhibitor 10 mg/m²/day and 1200 mg/m²/day BID. For example, EZH2 inhibitors of the disclosure may be administered at a dose of 100, 120, 140, 150, 160, 200, 240, 250, 260, 300, 320, 350, 380, 400, or 600 mg/m² BID.

In some embodiments, a pharmaceutical composition or dosage form comprising an EZH2 inhibitor is provided that is formulated for parenteral or enteral administration, for example, as an oral tablet, suspension, or solution. In some such embodiments, the pharmaceutical composition or dosage form may be suitable for administration of the EZH2 inhibitor at a dose of 50%, 60%, 70%, 80%, 90%, or any percentage in between of a value of an area under the curve (AUC) of a steady state plasma and/or CSF concentration (AUCss) of an EZH2 inhibitor, wherein the AUCss is determined following administration of the EZH2 inhibitor to an adult subject at a dose of between 10 mg/kg/day and 1600 mg/kg/day BID.

In some embodiments, a pharmaceutical composition or dosage form comprising an EZH2 inhibitor is provided that is formulated for parenteral or enteral administration, for example, as an oral tablet, suspension, or solution. In some such embodiments, the pharmaceutical composition or dosage form may be suitable for administration of the EZH2 inhibitor at a dose of between 230 mg/m² and 600 mg/m², inclusive of the endpoints. EZH2 inhibitors of the disclosure may be administered at a dose of between 300 mg/m² and 600 mg/m². EZH2 inhibitors of the disclosure may be administered at a dose of between 230 mg/m² and 305 mg/m², inclusive of the endpoints. EZH2 inhibitors of the disclosure may be administered at a dose of 240 mg/m². EZH2 inhibitors of the disclosure may be administered at a dose of 300 mg/m². EZH2 inhibitors of the disclosure may be administered once or twice per day (BID). For example, EZH2 inhibitors of the disclosure may be administered at a dose of between 230 mg/m² and 600 mg/m² BID, inclusive of the endpoints.

In some embodiments, a pharmaceutical composition or dosage form comprising an EZH2 inhibitor is provided that is suitable for administration of the EZH2 inhibitor at a dose of about 60% of the area under the curve (AUC) at steady state (AUCss) following administration of 1600 mg twice a day to an adult subject. Accordingly, in some such embodiment, a pharmaceutical composition or dosage form is provided that is suitable for administration of the EZH2 inhibitor at a dose of about about 600 mg/m² per day or at least 600 mg/m² per day. In some embodiments, the pharmaceutical composition is suitable for administration to a pediatric subject.

In some embodiments, a pharmaceutical composition or dosage form comprising an EZH2 inhibitor is provided that is suitable for administration of the EZH2 inhibitor at a dose of about 80% of the area under the curve (AUC) at steady state (AUCss) following administration of 800 mg twice a day to an adult subject. Accordingly, in some such embodiment, a pharmaceutical composition or dosage form is provided that is suitable for administration of the EZH2 inhibitor at a dose of about about 390 mg/m² per day or at least 390 mg/m² per day. In some embodiments, the pharmaceutical composition is suitable for administration to a pediatric subject.

In some embodiments, the present disclosure provides pharmaceutical compositions and dosage forms comprising an EZH2 inhibitor that are suitable for administration to a pediatric subject, e.g., a subject between 6 months and 21 years of age, inclusive of the endpoints; between 1 year and 18 years of age, inclusive of the endpoints; 10 years of age or less; 5 years of age or less; between 6 months and 1 year of age, inclusive of the endpoints; about 1 year of age; about 2 years of age; about 3 years of age; about 4 years of age; about 5 years of age; about 6 years of age; about 7 years of age; about 8 years of age; about 9 years of age; about 10 years of age; about 11 years of age; about 12 years of age; about 13 years of age; about 14 years of age; about 15 years of age; about 16 years of age; about 17 years of age; about 18 years of age; about 19 years of age; about 20 years of age; or about 21 years of age. In some embodiments, a pharmaceutical composition or dosage form comprising an EZH2 inhibitor is provided that is suitable for administration to a subject that is at least 1, 2, 3, 4, 5, 6, 7, 8, 9, 10, or 12 years of age, and not more than 5, 6, 7, 8, 9, 10, 11, 12, 13, 14 ,15, 16, 17, 18, 19, 20, or 21 years of age, wherein every possible age range that can be formed with these values (e.g., at least 4 and not more than 12 years of age; or at least 10 and not more than 18 years of age).

The pharmaceutical compositions containing an EZH2 inhibitor of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof

Compositions suitable for oral administration generally include an inert diluent or an edible pharmaceutically acceptable carrier. In some embodiments, they can be enclosed in capsules, e.g., in gelatin capsules, or compressed into tablets. For the purpose of oral therapeutic administration, the active compound can be incorporated with excipients and used in the form of tablets, troches, or capsules. Compositions for oral administration can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds (e.g., EZH2 inhibitors of the disclosure) can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The EZH2 inhibitors of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The EZH2 inhibitors of the present disclosure can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p1-92, Elesevier, New York-Oxford (1985).

The EZH2 inhibitors, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

The dosage regimen can be daily administration (e.g. every 24 hours) of a compound of the present disclosure. The dosage regimen can be daily administration for consecutive days, for example, at least two, at least three, at least four, at least five, at least six or at least seven consecutive days. Dosing can be more than one time daily, for example, twice, three times or four times daily (per a 24 hour period). The dosing regimen can be a daily administration followed by at least one day, at least two days, at least three days, at least four days, at least five days, or at least six days, without administration.

Techniques for formulation and administration of the disclosed compounds of the disclosure can be found in Remington: the Science and Practice of Pharmacy, 19th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight.

Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

In order that the invention disclosed herein may be more efficiently understood, examples are provided below. It should be understood that these examples are for illustrative purposes only and are not to be construed as limiting the disclosure in any manner.

### Example 1

### Tazemetostat Descreases Medulloblastoma Cell Growth

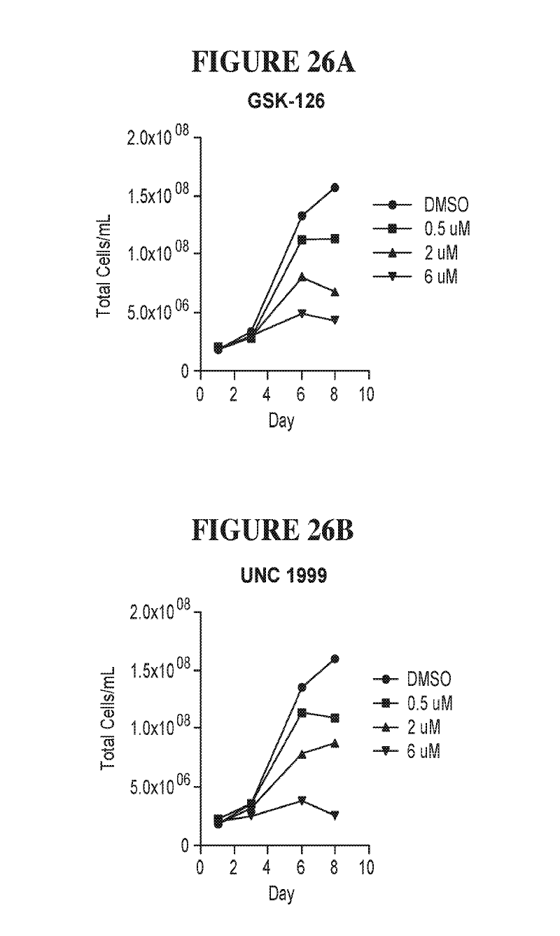

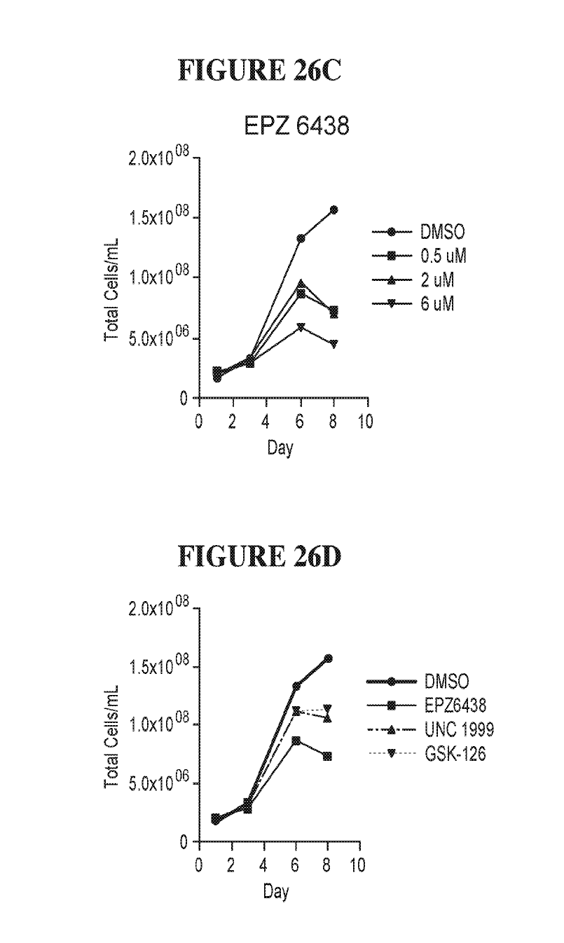

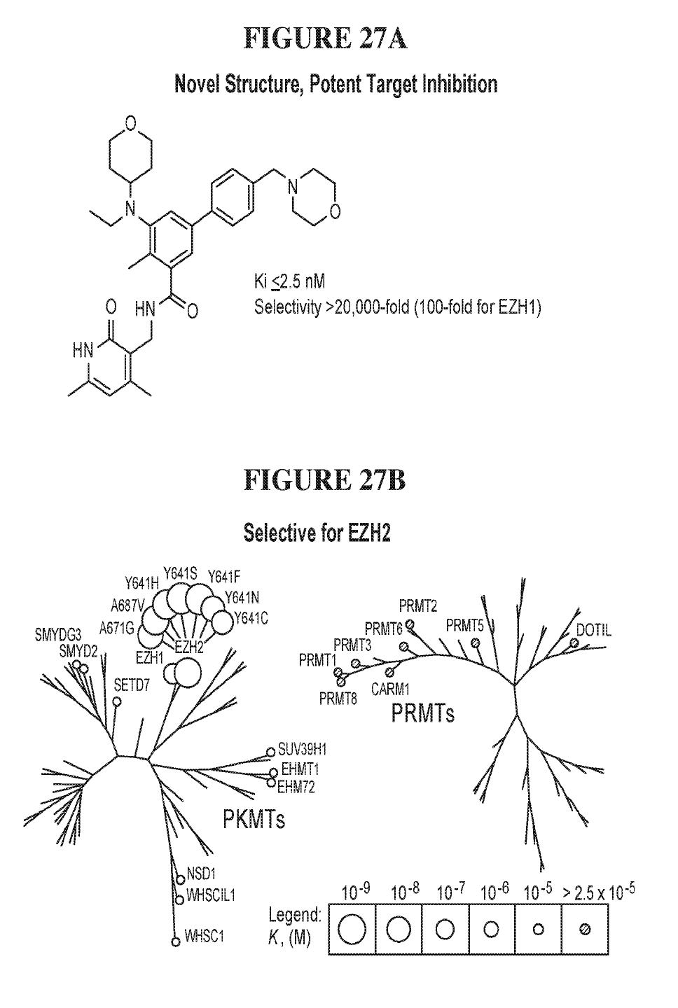

Medulloblastoma cells are treated with either a negative control (DMSO) or varying concentrations of tazemetostat (EPZ 6438): 0.5 μM, 2 μM and 6 μM. The total cells per milliliter of culture were counted each day for 10 days. While each tazemetostat treatment demonstrated a significant decrease on medulloblastoma cell growth compared to wild type (FIG. 26C), the effect was concentration dependent.

When compared to the efficacy of other small molecule EZH2 inhibitors, including GSK-126 and UNC 1999, Tazemetostat demonstrated a superior ability to decrease medulloblastoma cell growth (FIG. 26D).

### Example 2

### Tazemetostat Descreases Medulloblastoma Cell Growth In An Ex Vivo Slice Culture

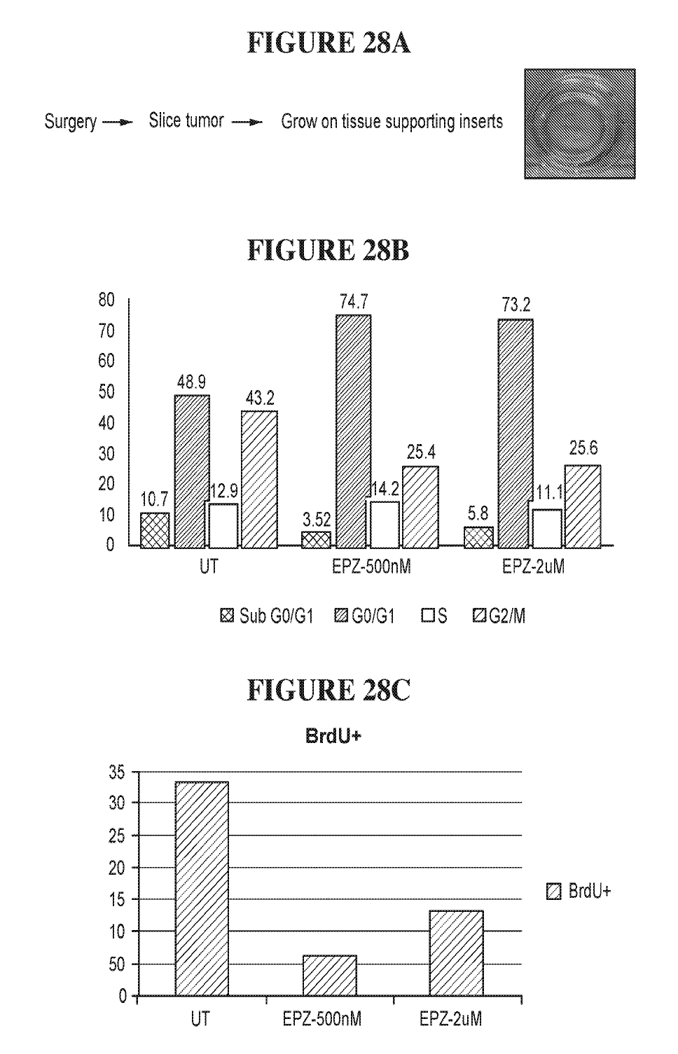

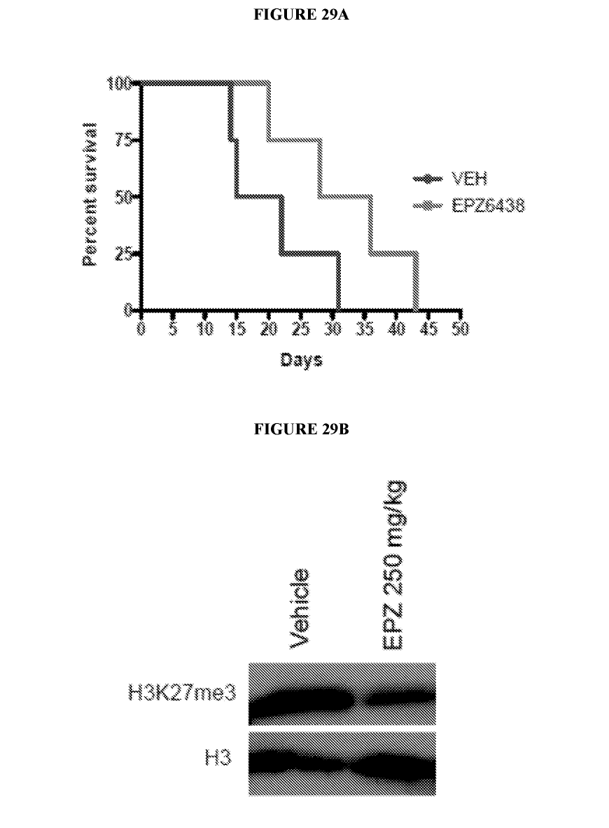

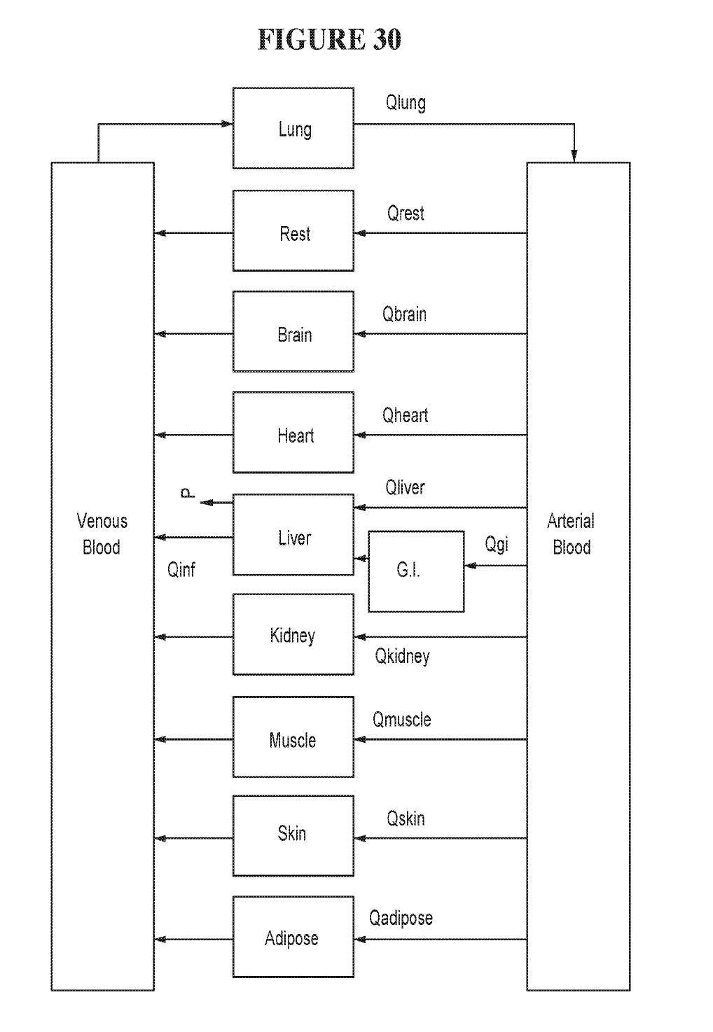

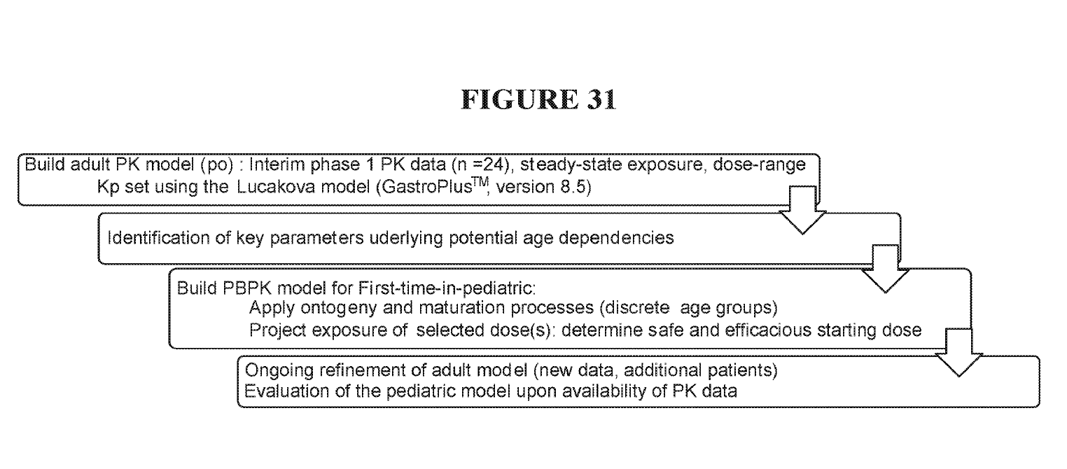

A 5 year old patient having medulloblastoma underwent surgery to remove a slice of tumor tissue for testing. The medulloblastoma slice was cultured ex vivo on tissue supporting inserts (FIG. 28A). Portions of the slice culture were untreated, treated with a lower concentration of tazemetostat (500 nM) or a higher concentration of tazemetostat (2 μM) for 4 days. Following the treatment period, the cells of the slice culture were treated with BrdU for 4 hours prior to disaggregation and sorting by flow cytometry.

FIG. 28B provides the results of the treatment by depicting the percent of cells in each of four cell cycle stages (sub G0/G1, Go/G1, S or G2/M) following each one of the treatment conditions. The data demonstrate that, compared to the untreated control, an increased proportion of medulloblastoma cells treated with tazemetostat are in the GO/G1 stage and a decreased proportion of medulloblastoma cells treated with tazemetostat are in the G2/M stage. The data indicate that treatment with tazemetostat inhibits proliferation/growth of medulloblastoma cells by interfering with cell division.

FIG. 28C confirms the results of FIG. 28B showing that the number of cells synthesizing DNA is significantly decreased in the tazemetostat-treated cells as evidenced by decreased incorporation of BrdU.

### Example 3

### Tazemetostat Pharmacokinetic (PK) Data from Human Phase 1 Clinical Trial: Steady-State PK Parameters in Adults

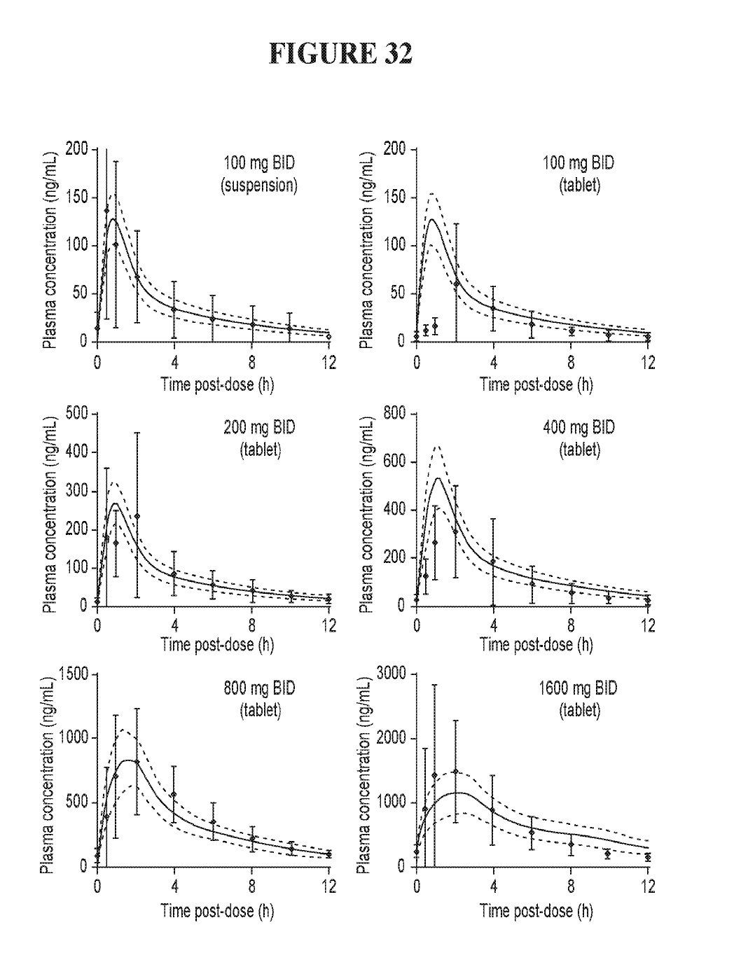

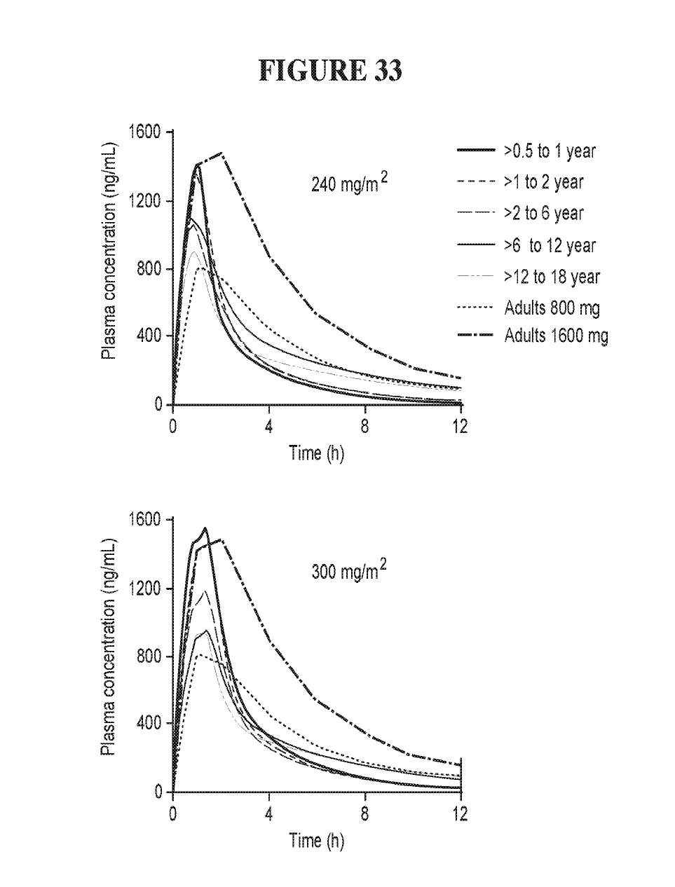

Tazemetostat pharmacokinetic (PK) data from the first in human phase 1 clinical trial study (CT. gov: NCT101897571), across a dose range of 100 mg (suspension) and 100, 200, 400, 800 and 1600 mg (tablet) p.o. twice daily (BID), together with in vitro data including plasma protein binding, blood partitioning, metabolic stability and P450 phenotyping were used to simulate adult exposures by physiologically-based pharmacokinetic (PBK) modeling (Gastroplus™ 8.5, Simulations Plus Inc.). A model fit for the adult exposures (n=24) adequately describes the time-concentration profiles of tazemetostat. This resulted in prediction of mean steady-state AUC₀₋ₜ and oral clearance (CL/F) with ±30% of the observed results across the dose ranges. In addition, mean steady-state Cₘₐₓ was predicted to ≤2-fold of the observed values, for both suspension and tablet formulations. The resultant model was used to simulate tazemetostat steady-state exposures in discrete pediatric age ranges of (6 month to 1 year (yr), >1-2 yrs, >2-6 yrs, >6-12 yrs, >12-18 yrs) following BID administration of the oral suspension. In addition to the pediatric physiology, the simulations accounted for ontogeny in hematocrit, plasma protein levels and CYP expression. Using this exposure-based analysis, pediatric doses which afforded the target AUC (80% of the adult steady-state AUC₀₋ₜ at 800 mg or 300 mg/m² BID) were identified. On the body surface area normalized basis, the projected doses were comparable across the age range (1 to 18 years) from 270 to 305 mg/m² BID, with a slightly lower projected dose of 230 mg/m² BID for the 6 months to 1 year old group. As the projected doses by the age were comparable, population simulations were performed to determine the corresponding exposures for each age range at a fixed 300 mg/m² BID dose. At this dose, mean steady-state Cₘₐₓ was projected to range between 895 ng/mL and 1550 ng/mL (110% to 190% of Cₘₐₓ at 800 mg BID in adult), but within the safe and efficacious exposure range defined in adults at doses up to 1600 mg BID (FIGS. 32 and 33).

The time-concentration profiles of tazemetostat administered orally at doses 100-1600 mg BID in adults were well predicted using a PBPK model, resulting in prediction of mean steady-state AUC₀₋ₜ and oral clearance (CL/F) within ±30% of the observed results across the dose range. Using this exposure-based analysis, pediatric starting doses which afforded AUCss within the safe and efficacious exposure range defined in adults were identified. For children 1-18 years of age, the starting dose of 240 mg/m² was predicted to result in 64% and 36% of the mean steady-state (AUCss) observed for adults at 800 mg and 1600 mg doses, respectively. For children 6 months to 1 year of age, the starting dose of 240 mg/m² was predicted to result in 80% and 45% of the mean AUCss observed for adults at 800 mg and 1600 mg doses, respectively. The data demonstrated the prospective application of PBPK early in clinical development to support clinical trial design in pediatric patients (see Tables 1 and 2).

TABLE 1
Drug-Specific data input parameters used in PBPK model.

| Data inputs | Parameters |
|---|---|
| Physicochemical | pKa |
| In vitro ADME | Plasma protein binding (fᵤ) |
| | Blood:plasma partition ratio |
| | Permeability (LLC-PK1 cells) |
| | HLM clearance (Kₘ and Vₘₐₓ) |
| | CYP phenotyping |
| Clinical PK | Renal Clearance (fᵤ * GFR) |

TABLE 2
Ratio of predicted versus observed tazemetostat steady-state PK parameters in adults.

| | Cₘₐₓ (ng/mL) | | | AUC₀₋ₜ (ng*h/mL) | | | CL/F (L/h) | | |
|---|---|---|---|---|---|---|---|---|---|
| BID dose | | | Predicted/ | | | Predicted/ | | | Predicted/ |
| (mg) | Observed | Predicted | Observed | Observed | Predicted | Observed | Observed | Predicted | Observed |
| 100 (n = 6) | 99 | 147 | 1.5 | 348 | 442 | 1.3 | 309 | 225 | 0.73 |
| 200 (n = 3) | 237 | 276 | 1.2 | 1010 | 950 | 0.94 | 198 | 208 | 1.1 |
| 400 (n = 3) | 313 | 586 | 1.9 | 1510 | 2010 | 1.3 | 264 | 198 | 0.75 |
| 800 (n = 6) | 818 | 938 | 1.1 | 4630 | 4140 | 0.89 | 152 | 190 | 1.2 |
| 1600 (n = 6) | 1480 | 1230 | 0.83 | 7880 | 8200 | 1.0 | 180 | 193 | 1.1 |

The details of one or more embodiments of the disclosure are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs.

All patents and publications cited in this specification are incorporated by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow. Where names of cell lines or genes are used, abbreviations and names conform to the nomenclature of the American Type Culture Collection (ATCC) or the National Center for Biotechnology Information (NCBI), unless otherwise noted or evident from the context.

The invention disclosed herein can be embodied in other specific forms without departing from the spirit or essential characteristics thereof The foregoing description has been presented only for the purposes of illustration and is not intended to limit the invention to the precise form disclosed, but by the claims appended hereto, and all changes that come within the meaning and range of equivalency of the claims are intended to be embraced therein.

1. A method of treating a an INI1-deficient tumor in a subject in need thereof comprising administering to the subject a therapeutically-effective amount of an enhancer of a zeste homolog 2 (EZH2) inhibitor.

2. The method of claim 1, wherein the EZH2 inhibitor comprises 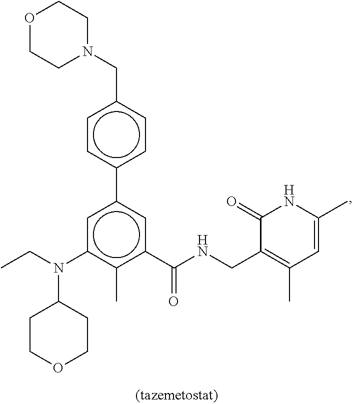
or a pharmaceutically-acceptable salt thereof.

3.-7. (canceled)

8. The method of claim 1, wherein the EZH2 inhibitor is administered orally.

9. (canceled)

10. The method of claim 8, wherein the EZH2 inhibitor is administered at a dose of between 10 mg/kg/day and 1600 mg/kg/day.

11. The method of claim 10, wherein the EZH2 inhibitor is administered at a dose of about 100, 200, 400, 800, or 1600 mg/kg/day.

12. The method of claim 11, wherein the EZH2 inhibitor is administered at a dose of about 800 mg/kg/day.

13. (canceled)

14. The method of claim 1, wherein the EZH2 inhibitor is formulated for administration to cerebral spinal fluid (CSF), wherein the EZH2 inhibitor is administered to cerebral spinal fluid by an intraspinal, an intracranial, an intrathecal or an intranasal route.

15. (canceled)

16. The method of claim 8, wherein the EZH2 inhibitor is administered at a dose of between 230 mg/m² and 600 mg/m² twice per day (BID), inclusive of the endpoints, or at a dose of between 230 mg/m² and 305 mg/m² twice per day (BID), inclusive of the endpoints.

17. (canceled)

18. The method of claim 8, wherein the EZH2 inhibitor is administered at a dose of 240 mg/m² twice per day (BID), or at a dose of 300 mg/m² twice per day (BID).

19. (canceled)

20. The method of claim 8, wherein the EZH2 inhibitor is administered at a dose of about 60% of the area under the curve (AUC) at steady state (AUCss) following administration of 1600 mg twice a day to an adult subject.

21. The method of claim 13 or 20, wherein the EZH2 inhibitor is administered at a dose of about 600 mg/m² per day.

22. (canceled)

23. The method of claim 8, wherein the EZH2 inhibitor is administered at a dose of about 80% of the area under the curve (AUC) at steady state (AUCss) following administration of 800 mg twice a day to an adult subject.

24. The method of claim 23, wherein the EZH2 inhibitor is administered at a dose of about 390 mg/m² twice per day (BID).

25. (canceled)

26. The method of claim 8, wherein the EZH2 inhibitor is administered at a dose of between 300 mg/m² and 600 mg/m² twice per day (BID).

27. The method of claim 1, wherein the EZH2 inhibitor is administered twice per day (BID).

28. The method of claim 1, wherein the subject is a pediatric subject.

29. The method of claim 28, wherein the subject is between 6 months and 21 years of age, inclusive of the endpoints, or between 1 year and 18 years of age, inclusive of the endpoints.

30. (canceled)

31. The method of claim 28, wherein the subject is 10 years of age or less or 5 years of age or less.

32. (canceled)

33. The method of claim 1, wherein treating comprises preventing and/or inhibiting proliferation of an INI1-deficient tumor cell.

34. A method of treating an INI1-deficient tumor in a subject in need thereof comprising administering to the subject a therapeutically-effective amount of tazemetostat,
wherein the therapeutically effective amount is at least 300 mg/m² twice per day (BID), and
wherein the subject is between 6 months and 21 years of age, inclusive of the endpoints.
